(19) United States
(12) Patent Application Publication
(10) Pub. No.: US 2007/0066525 A1
(43) Pub. Date: Mar. 22, 2007

(54) Compositions and therapeutic methods using morphogenic proteins

(76) Inventors: John C. Lee, San Antonio, TX (US); Lee-Chuan C. Yeh, San Antonio, TX (US)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 11/499,775

(22) Filed: Aug. 3, 2006

Related U.S. Application Data
(63) Continuation of application No. PCT/US04/03440, filed on Feb. 4, 2004.

(51) Int. Cl. A61K 38/18

(52) U.S. Cl. 514/12

(57) ABSTRACT

The present invention provides pharmaceutical compositions comprising at least two morphogenic proteins, particularly those belonging to the BMP protein family. This invention also provides implantable morphogenic devices comprising a first morphogenic protein and a second morphogenic protein combination disposed within a carrier, that are capable of inducing tissue formation. Methods for inducing local tissue formation from a progenitor cell in a mammal using those compositions and devices are also provided. Methods for improving the tissue inductive activity in a mammal at a target locus using the morphogenic proteins, nucleic acids encoding them, vectors comprising the nucleic acids encoding them and cells comprising the vectors are also provided.

This application is a continuation of International application No. PCT/US04/03440, filed Feb. 4, 2004, the entire disclosure of which is incorporated by reference herein.

### BACKGROUND OF THE INVENTION

Osteogenic proteins were defined originally as an activity present in mammalian bone extracts, presumably active during growth and natural bone healing, capable of inducing a developmental cascade leading to cartilage and endochondral bone accumulation when implanted in vivo. This developmental cascade includes mesenchymal cell recruitment and proliferation, progenitor cell differentiation, cartilage calcification, vascular invasion, bone formation, remodeling and marrow differentiation (Reddi, Collagen Rel. Res., 1, pp. 209-26 (1981)).

The factors in bone matrix that induce endochondral bone differentiation can be dissociatively extracted and reconstituted with inactive collagenous matrix to restore full bone inductive activity (Reddi, Proc. Natl. Acad. Sci. USA, 78, pp. 7599-7603 (1981)). This provides an experimental method for assaying protein extracts for their ability to induce endochondral bone formation in vivo. Using this reconstitution assay, a variety of related osteogenic proteins have been isolated from several mammalian species that are capable of inducing bone and cartilage formation in cross-species implants (Sampath and Reddi, Proc. Natl. Acad. Sci. USA, 80, pp. 6591-95 (1983)). The active factor or factors that promote this activity have been referred to in the literature most commonly as bone morphogenetic proteins (BMPs) and osteogenic proteins (OPs).

Osteogenic and bone morphogenetic proteins represent a family of structurally and functionally related morphogenic proteins belonging to the Transforming Growth Factor-Beta (TGF-β) superfamily (see below). The TGF-β superfamily, in turn, represents a large number of evolutionarily conserved proteins with diverse activities involved in growth, differentiation and tissue morphogenesis and repair. BMPs and osteogenic proteins, as members of the TGF-β superfamily, are expressed as secretory polypeptide precursors which share a highly conserved bioactive cysteine domain located near their C-termini. Another feature of many of the BMP family proteins is their propensity to form homo- and heterodimers.

Many morphogenic proteins belonging to the BMP family have now been described. Some have been isolated using purification techniques coupled with bioassays such as the one described above. Others have been identified and cloned by virtue of DNA sequence homologies within conserved regions that are common to the BMP family. These homologs are referred to as consecutively-numbered BMPs whether or not they have demonstrable osteogenic activity. Using an alternative approach, synthetic OPs having osteogenic activity have been designed using amino acid consensus sequences derived from sequence comparisons between naturally-derived OPs and BMPs (see below; Oppermann et al., U.S. Pat No. 5,324,819).

While several of the earliest members of the BMP family were osteogenic proteins identified by virtue of their ability to induce new cartilage and bone, the search for BMP-related genes and gene products in a variety of species has revealed new morphogenic proteins, some of which have different or additional tissue-inductive capabilities. For example, BMP-12 and BMP-13 (identified by DNA sequence homology) reportedly induce tendon/ligament-like tissue formation in vivo (WO 95/16035). Several BMPs can induce neuronal cell proliferation and promote axon regeneration (WO 95/05846). And some BMPs that were originally isolated on the basis of their osteogenic activity also have neural inductive properties (Liem et al., Cell, 82, pp. 969-79 (1995)).

Other BMPs have been reported to induce other tissues. For example, CDMP-1, -2 and -3 have been shown to affect several skeletal processes, including joint formation, tendon/ligament repair and endochondral ossification (Francis-West et al., Cell Tissue Res, 296, pp. 111-119 (1999); Merino et al., Dev Biol, 206, pp. 33-45 (1999); Storm and Kingsley, Dev Biol, 209, pp. 11-27 (1999); Aspenberg and Forslund, Acta Orthop Scand, 70,. pp. 51-54 (1999); Aspenberg and Forslund, Scand J Med Sci Sports, 10, pp. 372-375 (2000); Lou et al., J Orthop Res, 19, pp. 1199-1202 (2001); Rickert et al., Growth Factors, 19, pp. 115-126 (2001); Wolfman et al., J Clin Invest, 100, pp. 321-330 (1997)). CDMP-1 and -2 stimulate osteogenic differentiation of bone marrow cells (Gruber et al., Cytokine, 12, pp. 1630-1638 (2000)) and of periosteum-derived cells (Gruber et al., Endocrinology, 142, pp. 2087-2094 (2001)). CDMP-1 and -2 are capable of inducing cartilage and bone formation when implanted ectopically in intramuscular sites (Hotten et al., Growth Factors, 13, pp. 65-74 (1996); Erlacher et al., J Bone Miner Res, 13, pp. 383-392 (1998); Spiro et al., Biochem Soc Trans, 28, pp. 362-368 (2000)). It thus appears that osteogenic proteins and other BMPs may have a variety of potential tissue inductive capabilities whose final expression may depend on a complex set of developmental and environmental cues. These osteogenic, BMP and BMP-related proteins are referred to herein collectively as morphogenic proteins.

The activities described above, and other as yet undiscovered tissue inductive properties of the morphogenic proteins belonging to the BMP family are expected to be useful for promoting tissue regeneration in patients with traumas caused, for example, by injuries or degenerative disorders. Implantable osteogenic devices comprising mammalian osteogenic protein for promoting bone healing and regeneration have been described (see, e.g., Oppermann et al., U.S. Pat. No. 5,354,557). Some osteogenic devices comprise osteogenic protein dispersed in porous, biocompatible matrices. These naturally-derived or synthetic matrices typically allow osteogenic protein to diffuse out of the matrix into the implantation site and permit influx and efflux of cells. Osteogenic protein induces the progenitor cells to differentiate and proliferate. Progenitor cells may migrate into the matrix and differentiated cells can move out of the porous matrix into the implant site. Osteogenic cells may also utilize the matrix as a physical scaffold for osteoconduction. Similar devices have been described for delivering BMPs for tendon/ligament-like tissue regeneration. Osteogenic protein-coated prosthetic devices which enhance the bond strength between the prosthesis and existing bone have also been described (Rueger et al., U.S. Pat. No. 5,344,654, incorporated herein by reference).

The availability of large amounts of purified and highly active morphogenic proteins would revolutionize orthopedic medicine, certain types of plastic surgery, dental and various periodontal and craniofacial reconstructive procedures, and procedures generally involving bone, cartilage, tendon and ligament. Many of the mammalian OP- and BMP-encoding genes are now cloned and may be recombinantly expressed as active homo- and heterodimeric proteins in a variety of host systems, including bacteria. The ability to recombinantly produce active forms of morphogenic proteins such as OPs and BMPs, including variants, fragments thereof and mutants with increased bioactivities, make potential therapeutic treatments using morphogenic proteins feasible.

Given the large number of potential therapeutic uses for morphogenic proteins in treating a variety of different tissues and tissue-types, there is a need for highly active forms of morphogenic proteins. It would, thus, be desirable to increase the tissue inductive properties of morphogenic proteins. With increased tissue inductive activity, treatment with a morphogenic protein, even on large scales, could induce tissue formation more rapidly, or tissue induction could be achieved using reduced morphogenic protein concentrations.

### SUMMARY OF THE INVENTION

This invention is based on the discovery that morphogenic proteins possess tissue inductive activity and that the tissue inductive ability of a first morphogenic protein can be synergistically enhanced by a second morphogenic protein. The invention provides for a pharmaceutical composition comprising a first morphogenic protein, a second morphogenic protein different from the first morphogenic protein and a pharmaceutically acceptable carrier. In a preferred embodiment, the first and second morphogenic proteins independently include but are not limited to OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3 (BMP-12), CDMP-2 (BMP-13), CDMP-1 (BMP-14), BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, NEURAL, or fragments thereof. In some embodiments, at least one of the first and second morphogenic proteins comprises a dimeric protein having an amino acid sequence having at least 70% homology within the C-terminal 102-106 amino acids of human OP-1. In some embodiments, the first morphogenic protein is OP-1 or a fragment thereof and the second morphogenic protein is selected from the group consisting of CDMP-1, CDMP-2 and CDMP-3 and fragments thereof. In a preferred embodiment, the second morphogenic protein is CDMP-1 or a fragment thereof. In another preferred embodiment, the second morphogenic protein is CDMP-2 or a fragment thereof. In yet another preferred embodiment, the second morphogenic protein is CDMP-3 or a fragment thereof. In some embodiments, the pharmaceutical composition further comprises at least one additional morphogenic protein or fragment thereof.

In some embodiments, the second morphogenic protein is present in an amount sufficient to synergistically stimulate the first morphogenic protein. In some embodiments, the first morphogenic protein is present in an amount sufficient to synergistically stimulate the second morphogenic protein.

The invention also provides for an implantable device comprising an implantable biocompatible carrier, a first morphogenic protein according to the present invention and a second morphogenic protein different from the first morphogenic protein according to this invention. In some embodiments, the biocompatible carrier is a biocompatible matrix. In a preferred embodiment, the matrix is selected from the group consisting of demineralized, protein-extracted, protein-extracted, particulate, allogenic bone.

The invention also provides a method of improving the tissue inductive activity in a mammal of a first morphogenic protein capable of inducing tissue formation when accessible to a progenitor cell according to this invention by coadministering an effective amount of at least a second morphogenic protein different from the first morphogenic protein, according to this invention. In some embodiments, the second morphogenic protein synergistically improves the tissue inductive activity of the first morphogenic protein. In some embodiments, the tissue formation is selected from the group consisting of bone, cartilage, tendon and ligament formation.

The invention also provides for a method of inducing local tissue formation from a progenitor cell in a mammal comprising the step of implanting in the mammal a composition or a device according to this invention. In some embodiments, the first and second morphogenic proteins are each independently capable of inducing the a progenitor cell to form bone, cartilage, tendon or ligament. In some embodiments, the second morphogenic protein synergistically improves the tissue inductive activity of the first morphogenic protein.

The invention also provides for a method for improving the tissue inductive activity in a mammal of a first morphogenic protein capable of inducing tissue formation at a target locus by coadministering an effective amount of a second morphogenic protein different from the first morphogenic protein, the method comprising administering to the target locus a nucleic acid encoding the first morphogenic protein and a nucleic acid encoding a second morphogenic protein.

The invention also provides for a method of improving the tissue inductive activity in a mammal of a first morphogenic protein capable of inducing tissue formation at a target locus by coadministering an effective amount of a second morphogenic protein different from the first morphogenic protein, the method comprising administering to the target locus a vector comprising a nucleic acid encoding the first morphogenic protein operably linked to an expression control sequence and a vector comprising a nucleic acid encoding a second morphogenic protein operably linked to an expression control sequence.

The invention also provides for a method for improving the tissue inductive activity in a mammal of a first morphogenic protein capable of inducing tissue formation at a target locus by coadministering an effective amount of a second morphogenic protein different from the first morphogenic protein, the method comprising administering to the target locus a cell comprising a vector comprising a nucleic acid encoding the first morphogenic protein operably linked to an expression control sequence and a cell comprising a vector comprising a nucleic acid encoding a second morphogenic protein operably linked to an expression control sequence.

In some embodiments, the first morphogenic protein and second morphogenic protein are administered simultaneously to the target locus. In some embodiments, the first morphogenic protein and second morphogenic protein are administered separately to the target locus.

In some embodiments, the nucleic acids encoding the first morphogenic protein and the second morphogenic protein are in the same vector. In some embodiments, the nucleic acids encoding the first morphogenic protein and the second morphogenic protein are in separate vectors. In some embodiments, the vectors comprising the nucleic acids encoding the first morphogenic protein and the second morphogenic protein are in the same cell. In some embodiments, the vectors comprising the nucleic acids encoding the first morphogenic protein and the second morphogenic protein are in separate cells.

### BRIEF DESCRIPTION OF THE DRAWINGS

FIG. 1. Effect of CDMP-1 on OP-1-stimulated alkaline phosphatase activity in C2C12 cells. Alkaline phosphatase activity was measured in C2C12 cells treated with 100 ng/ml OP-1 and increasing concentrations of CDMP-1 (50, 100 and 200 ng/ml) for 5 or 7 days. Results are normalized to the alkaline phosphatase activity in C2C12 cells treated with solvent-treated vehicle control alone. Values are means of three independent experiments (with six wells/treatment condition).

FIG. 2. Effect of CDMP-2 on OP-1-stimulated alkaline phosphatase activity in C2C12 cells. Alkaline phosphatase activity was measured in C2C12 cells treated with 100 ng/ml OP-1 and increasing concentrations of CDMP-2 (50, 100 and 200 ng/ml) for 5 or 7 days. Results are normalized to the alkaline phosphatase activity in C2C12 cells treated with solvent-treated vehicle control alone. Values are means of three independent experiments (with six wells/treatment condition).

FIG. 3. Effects of CDMP-3 on OP-1-stimulated alkaline phosphatase activity in C2C12 cells. Alkaline phosphatase activity was measured in C2C12 cells treated with 100 ng/ml OP-1 and increasing concentrations of CDMP-3 (50, 100 and 200 ng/ml) for 5 or 7 days. Results are normalized to the alkaline phosphatase activity in C2C12 cells treated with solvent-treated vehicle control alone. Values are means of three independent experiments (with six wells/treatment condition).

FIG. 4. Effects of CDMP-1, CDMP-2 and CDMP-3 on alkaline phosphatase staining in C2C12 cells. Alkaline phosphatase (AP) activity in C2C12 cells treated with solvent control, CDMP-1, CDMP-2 or CDMP-3 alone (50 ng/ml and 100 ng/ml), OP-1 alone (100 ng/ml) and the combination of OP-1 (100 ng/ml) and CDMP-1 (50 ng/ml and 100 ng/ml) was detected using a cytochemical kit. The intensity of the stain is proportional to the AP activity.

FIG. 5. Effects of CDMP-1, CDMP-2 and CDMP-3 on OP-1-induced osteocalcin (OC), MyoD and scleraxis mRNA expression in C2C12 cells. The mRNA expressions of OC, MyoD and scleraxis of C2C12 cells treated with 100 ng/mL OP-1 in the absence or presence of 200 ng/mL of CDMP-1, CDMP-2 or CDMP-3 for 5 days were measured by Northern blot analysis using ³²P-labeled cDNA probes. The mRNA expression of 18S rRNA from these cells was also measured by Northern blot analysis using an oligonucleotide probed for 18S rRNA. Representative images (phosphorimages) of mRNA expressions of OC, MyoD, scleraxis and 18S rRNA are presented. Lanes 1-8 represent the mRNA expression levels of OC, MyoD, scleraxis and 18S rRNA in C2C12 cells treated for 5 days under the varying conditions.

FIG. 6. Effects of CDMP-1, CDMP-2 and CDMP-3 on (A) OP-1-induced osteocalcin (OC), (B) MyoD and (C) scleraxis mRNA expression in C2C12 cells. The mRNA expressions of OC, MyoD and scleraxis of treated C2C12 cells from FIG. 5 (Example 5) were quantitatively analyzed using the ImageQuant software. The mRNA expression levels were normalized to the 18S rRNA levels. The normalized mRNA levels were then compared to that in the same day control. Values are means of four independent experiments.

FIG. 7. The nucleotide (A) and predicted amino acid sequence (B) encoded by the full length human CDMP-1.

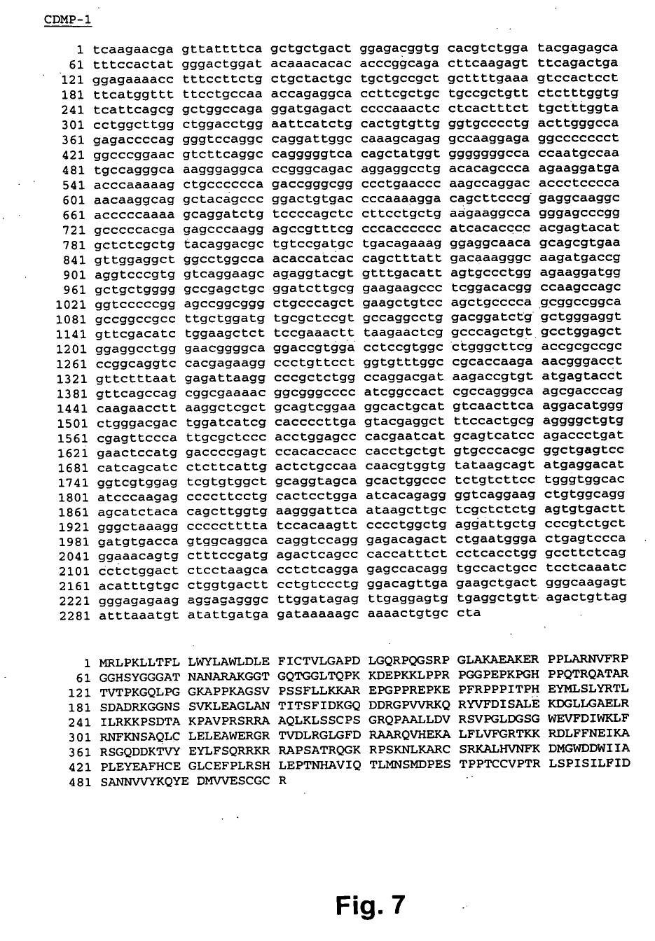

FIG. 8. The nucleotide (A) and predicted amino acid sequence (B) encoded by the full length murine CDMP-2.

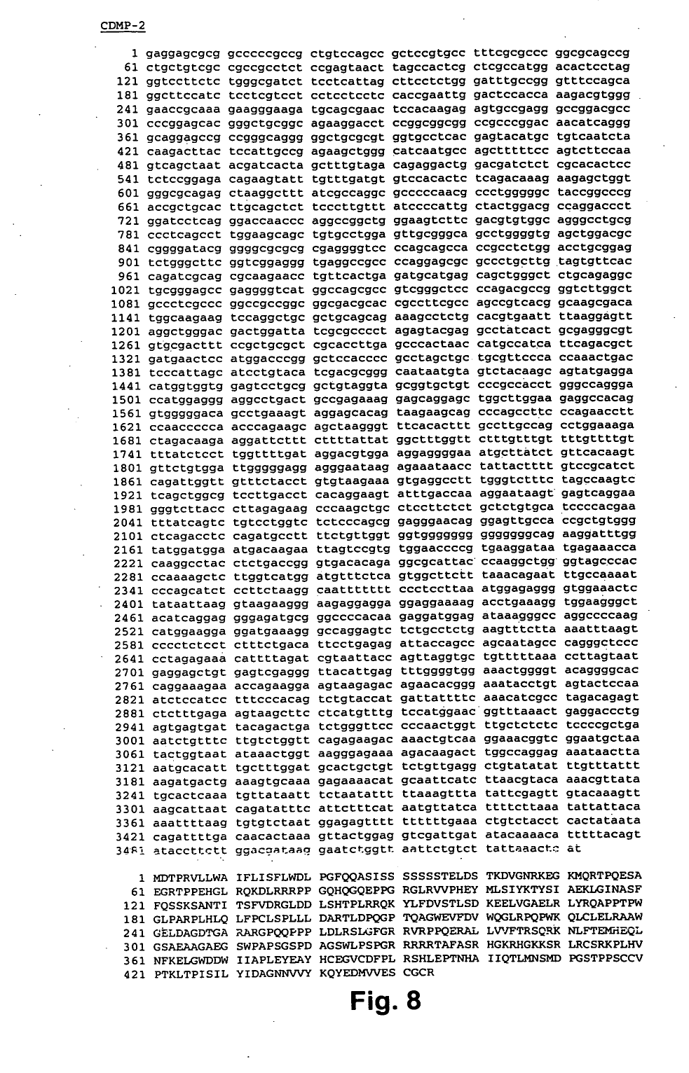

FIG. 9. The nucleotide (A) and predicted amino acid sequence (B) encoded by the full length human CDMP-3.

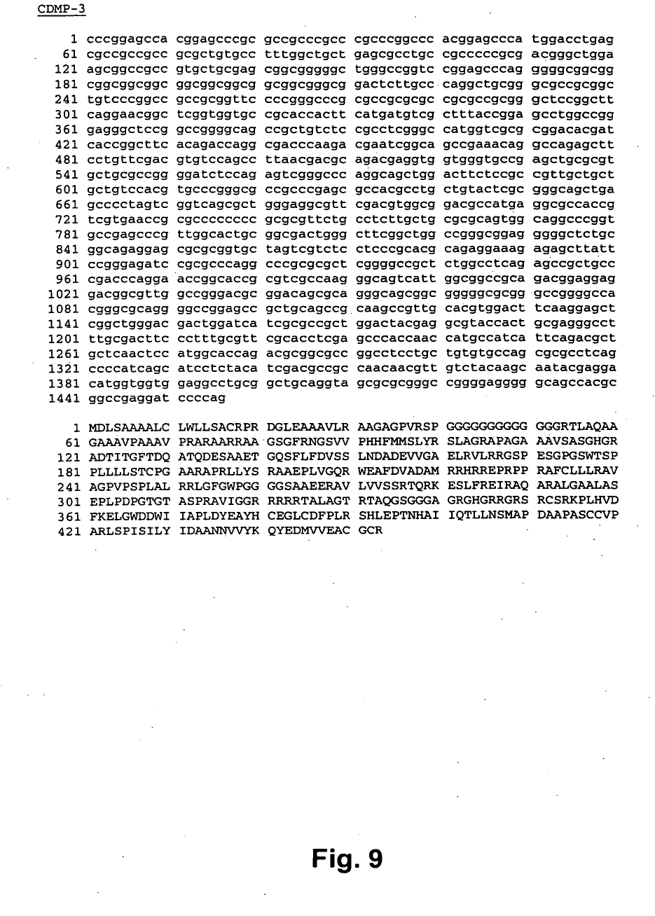

FIG. 10. The nucleotide (A) and predicted amino acid sequence (B) encoded by the full length human OP-1. Mature OP-1 corresponds to residues 293 to 431.

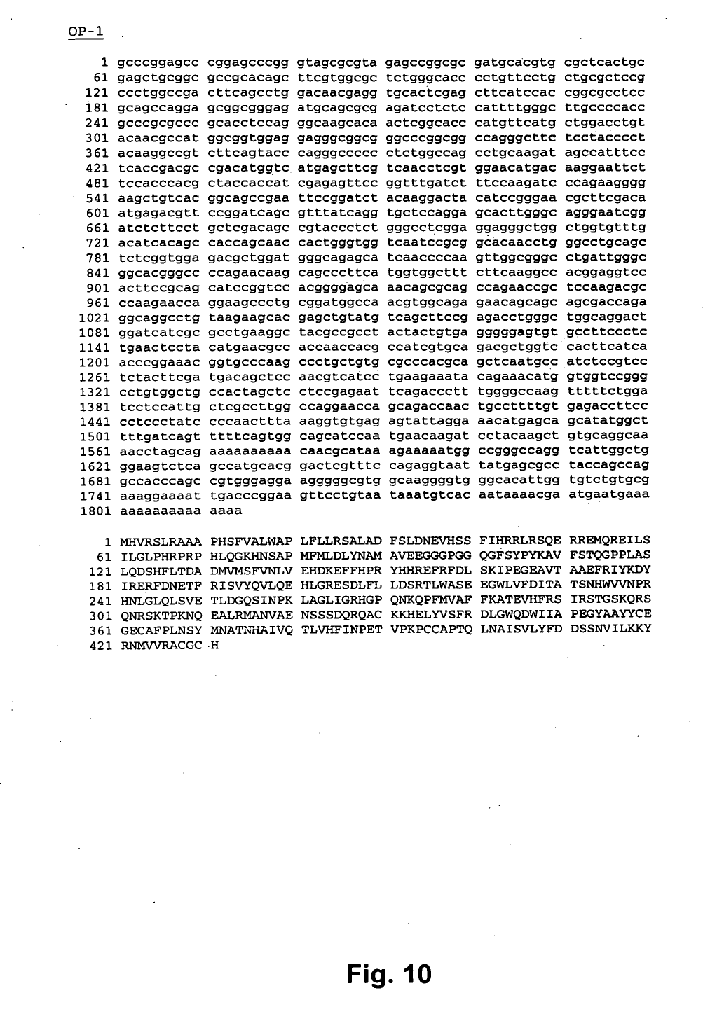

### DETAILED DESCRIPTION OF THE INVENTION

In order that the invention herein described may be fully understood, the following terms and definitions are provided herein.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention. All publications and other references mentioned herein are incorporated by reference in their entirety. In case of conflict, the present specification, including definitions will control. The materials, methods and examples are illustrative only and not intended to be limiting.

Throughout the specification, the word “comprise” or variations such as “comprises” or “comprising” will be understood to imply the inclusion of a stated integer or groups of integers but not the exclusion of any other integer or group of integers.

“Amino acid sequence homology” is understood to include both amino acid sequence identity and similarity. Homologous sequences share identical and/or similar amino acid residues, where similar residues are conservative substitutions for, or “allowed point mutations” of, corresponding amino acid residues in an aligned reference sequence. Thus, a candidate polypeptide sequence that shares 70% amino acid homology with a reference sequence is one in which any 70% of the aligned residues are either identical to, or are conservative substitutions of, the corresponding residues in a reference sequence. Certain particularly preferred morphogenic polypeptides share at least 60%, and preferably 70% amino acid sequence identity with the C-terminal 102-106 amino acids, defining the conserved seven-cysteine domain of human OP-1, BMP-2, and related proteins.

Amino acid sequence homology can be determined by methods well known in the art. For instance, to determine the percent homology of a candidate amino acid sequence to the sequence of the seven-cysteine domain, the two sequences are first aligned. The alignment can be made with, e.g., the dynamic programming algorithm described in Needleman et al., J. Mol. Biol., 48, pp. 443 (1970), and the Align Program, a commercial software package produced by DNAstar, Inc. The teachings of these references are incorporated herein by reference. An initial alignment can be refined by comparison to a multi-sequence alignment of a family of related proteins. Once the alignment is made and refined, a percent homology score is calculated. The aligned amino acid residues of the two sequences are compared sequentially for their similarity to each other. Similarity factors include similar size, shape and electrical charge. One particularly preferred method of determining amino acid similarities is the PAM250 matrix described in Dayhoff et al., Atlas of Protein Sequence and Structure, 5, pp. 345-352 (1978 & Supp.), which is incorporated herein by reference. A similarity score is first calculated as the sum of the aligned pairwise amino acid similarity scores. Insertions and deletions are ignored for the purposes of percent homology and identity. Accordingly, gap penalties are not used in this calculation. The raw score is then normalized by dividing it by the geometric mean of the scores of the candidate sequence and the seven-cysteine domain. The geometric mean is the square root of the product of these scores. The normalized raw score is the percent homology.

“Conservative substitutions” refers to residues that are physically or functionally similar to the corresponding reference residues. That is, a conservative substitution and its reference residue have similar size, shape, electric charge, chemical properties including the ability to form covalent or hydrogen bonds, or the like. Preferred conservative substitutions are those fulfilling the criteria defined for an accepted point mutation in Dayhoff et al., supra. Examples of conservative substitutions are substitutions within the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine. The term “conservative variant” or “conservative variation” also includes the use of a substituting amino acid residue in place of an amino acid residue in a given parent amino acid sequence, where antibodies specific for the parent sequence are also specific for, i.e., “cross-react” or “immuno-react” with, the resulting substituted polypeptide sequence.

The term “biocompatible” refers to a material that does not elicit detrimental effects associated with the body's various protective systems, such as cell and humoral-associated immune responses, e.g., inflammatory responses and foreign body fibrotic responses. The term biocompatible also implies that no specific undesirable cytotoxic or systemic effects are caused by the material when it is implanted into the patient.

The term “bone morphogenetic protein (BMP)” refers to a protein belonging to the BMP family of the TGF-β superfamily of proteins (BMP family) based on DNA and amino acid sequence homology. A protein belongs to the BMP family according to this invention when it has at least 50% amino acid sequence identity with at least one known BMP family member within the conserved C-terminal cysteine-rich domain which characterizes the BMP protein family. Preferably, the protein has at least 70% amino acid sequence identity with at least one known BMP family member within the conserved C-terminal cysteine-rich domain. Members of the BMP family may have less than 50% DNA or amino acid sequence identity overall.

The term “morphogenic protein” refers to a protein having morphogenic activity (see below). Preferably a morphogenic protein of this invention comprises at least one polypeptide belonging to the BMP protein family. Morphogenic proteins may be capable of inducing progenitor cells to proliferate and/or to initiate differentiation pathways that lead to cartilage, bone, tendon, ligament, or other types of tissue formation depending on local environmental cues, and thus morphogenic proteins may behave differently in different surroundings. For example, an osteogenic protein may induce bone tissue at one treatment site and cartilage tissue at a different treatment site. Morphogenic proteins include full length proteins as well as fragments thereof.

The term “osteogenic protein (OP)” refers to a morphogenic protein that is capable of inducing a progenitor cell to form cartilage and/or bone. The bone may be intramembranous bone or endochondral bone. Most osteogenic proteins are members of the BMP protein family and are thus also BMPs. As described elsewhere herein, the class of proteins is typified by human osteogenic protein (hOP-1). Other osteogenic proteins useful in the practice of the invention include but are not limited to, osteogenically active forms of OP-1, OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3 (BMP-12), CDMP-2 (BMP-13), CDMP-1 (BMP-14), BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, NEURAL, conservative amino acid sequence variants thereof having osteogenic activity and fragments thereof. In one currently preferred embodiment, osteogenic protein includes any one of: OP-1, OP-2, OP-3, BMP-2, BMP-4, BMP-5 , BMP-6, BMP-9, amino acid sequence variants and homologs thereof, including species homologs thereof and fragments thereof. Particularly preferred osteogenic proteins are those comprising an amino acid sequence having at least 70% homology with the C-terminal 102-106 amino acids, defining the conserved seven cysteine domain, of human OP-1, BMP-2, and related proteins. Certain preferred embodiments of the instant invention comprise the osteogenic protein, OP-1. As further described elsewhere herein, the osteogenic proteins suitable for use with this invention can be identified by means of routine experimentation using the art-recognized bioassay described by Reddi and Sampath (Sampath et al., Proc. Natl. Acad. Sci., 84, pp. 7109-13, incorporated herein by reference).

Proteins useful in this invention include eukaryotic proteins identified as osteogenic proteins (see U.S. Pat. 5,011,691, incorporated herein by reference), such as the OP-1, OP-2, OP-3 and CBMP-2 proteins, as well as amino acid sequence-related proteins, such as DPP (from Drosophila), Vg1 (from Xenopus), Vgr-1 (from mouse), GDF-1 (from humans, see Lee, PNAS, 88, pp. 4250-4254 (1991)), 60A (from Drosophila, see Wharton et al., PNAS, 88, pp. 9214-9218 (1991)), dorsalin-1 (from chick, see Basler et al., Cell, 73, pp. 687-702 (1993) and GenBank accession number L12032) and GDF-5 (from mouse, see Storm et al., Nature, 368, pp. 639-643 (1994)). The teachings of the above references are incorporated herein by reference. BMP-3 is also preferred. Additional useful proteins include biosynthetic morphogenic constructs disclosed in U.S. Pat. No. 5,011,691, incorporated herein by reference, e.g., COP-1, COP-3, COP-4, COP-5, COP-7 and COP-16, as well as other proteins known in the art. Still other proteins include osteogenically active forms of BMP-3b (see Takao, et al., Biochem . Biophys. Res. Comm ., 219, pp. 656-662 (1996)). BMP-9 (see WO 95/33830), BMP-15 (see WO 96/35710), BMP-12 (see WO 95/16035), CDMP-1 (see WO 94/12814), CDMP-2 (see WO 94/12814), BMP-10 (see WO 94/26893), GDF-1 (see WO 92/00382), GDF-10 (see WO095/10539), GDF-3 (see WO 94/15965) and GDF-7 (see WO095/01802). The teachings of the above references are incorporated herein by reference. BMPs (identified by sequence homology) must have demonstrable osteogenic activity in a functional bioassay to be osteogenic proteins according to this invention.

The term “first morphogenic protein” refers to a morphogenic protein as described above having morphogenic activity (see below), whose morphogenic activity can be enhanced in the presence of a second morphogenic protein. The first morphogenic protein may be endogenously expressed or expressed from a recombinant DNA molecule with a host cell.

The term “second morphogenic protein” refers to a morphogenic protein as described above having morphogenic activity (see below), that is capable of stimulating the tissue inductive activity of the first morphogenic protein. Preferably, the second morphogenic protein has a synergistic effect on the morphogenic activity of the first morphogenic protein.

The terms “morphogenic activity”, “inducing activity” and “tissue inductive activity” alternatively refer to the ability of an agent to stimulate a target cell to undergo one or more cell divisions (proliferation) that may optionally lead to cell differentiation. Such target cells are referred to generically herein as progenitor cells. Cell proliferation is typically characterized by changes in cell cycle regulation and may be detected by a number of means which include measuring DNA synthetic or cellular growth rates. Early stages of cell differentiation are typically characterized by changes in gene expression patterns relative to those of the progenitor cell, which may be indicative of a commitment towards a particular cell fate or cell type. Later stages of cell differentiation may be characterized by changes in gene expression patterns, cell physiology and morphology. Any reproducible change in gene expression, cell physiology or morphology may be used to assess the initiation and extent of cell differentiation induced by a morphogenic protein.

The term “fragment thereof” or “fragment” refers to a stretch of amino acid residues of at least about 5 amino acids. In some embodiments, this term refers to a stretch of amino acid residues at least about 10 amino acids. In other embodiments, it refers to a stretch of amino acid residues of at least about 15 to 20 amino acids. The fragments may be naturally derived or synthetically generated. To be active, any fragment must have sufficient length to display biological activity.

The term “synergistic interaction” refers to an interaction in which the combined effect of two agents is greater than the algebraic sum of each of their individual effects.

The present invention provides compositions comprising at least two morphogenic proteins and methods of use thereof. Accordingly, in one embodiment, the present invention provides a pharmaceutical composition comprising a) a first morphogenic protein, b) a second morphogenic protein different from the first morphogenic protein, and a pharmaceutically acceptable carrier.

The present invention also provides an implantable device comprising a) an implantable biocompatible carrier, b) a first morphogenic protein and c) a second morphogenic protein different from the first morphogenic protein.

The present invention further provides a method for improving the tissue inductive activity in a mammal of a first morphogenic protein capable of inducing tissue formation when accessible to a progenitor cell by coadministering an effective amount of at least a second morphogenic protein.

The invention also provides a method of inducing local tissue formation from a progenitor cell in a mammal comprising the step of implanting in the mammal a composition or morphogenic device as described herein.

The invention also provides a method for improving the tissue inductive activity in a mammal of a first morphogenic protein capable of inducing tissue formation at a target locus by coadministering an effective amount of a second morphogenic protein different from the first morphogenic protein, the method comprising the step of administering to the target locus nucleic acids encoding the first morphogenic protein and the second morphogenic protein or vectors comprising nucleic acids encoding the first morphogenic protein and the second morphogenic protein or cells comprising vectors comprising nucleic acids encoding the first morphogenic protein and the second morphogenic protein.

Morphogenic Proteins

The morphogenic proteins of this invention are capable of stimulating a progenitor cell to undergo cell division and differentiation, and that inductive activity is enhanced in the presence of at least a second morphogenic protein.

Bone Morphogenic Protein (BMP) Family

The BMP family, named for its representative bone morphogenic/osteogenic protein family members, belongs to the TGF-β protein superfamily. Of the reported “BMPs” (BMP-1 to BMP-18), isolated primarily based on sequence homology, all but BMP-1 remain classified as members of the BMP family of morphogenic proteins (Ozkaynak et al., EMBO J., 9, pp. 2085-93 (1990)).

The BMP family includes other structurally-related members which are morphogenic proteins, including the drosophila decapentaplegic gene complex (DPP) products, the Vg1 product of Xenopus laevis and its murine homolog, Vgr-1 (see, e.g., Massagué, Annu. Rev. Cell Biol., 6, pp. 597-641 (1990), incorporated herein by reference).

The Drosophila DPP and Xenopus Vg-1 gene products are 50% identical to each other (and 35-40% identical to TGF-β). Both the Dpp and Vg-1 products are morphogenic proteins that participate in early patterning events during embryogenesis of their respective hosts. These products appear to be most closely related to mammalian bone morphogenetic proteins BMP-2 and BMP-4, whose C-terminal domains are 75% identical with that of Dpp.

The C-terminal domains of BMP-3, BMP-5, BMP-6, and OP-1 (BMP-7) are about 60% identical to that of BMP-2, and the C-terminal domains of BMP-6 and OP-1 are 87% identical. BMP-6 is likely the human homolog of the murine Vgr-1 (Lyons et al., Proc. Natl. Acad . Sci. U.S.A., 86, pp. 4554-59 (1989)); the two proteins are 92% identical overall at the amino acid sequence level (U.S. Pat. No. 5,459,047, incorporated herein by reference). BMP-6 is 58% identical to the Xenopus Vg-1 product.

The naturally occurring bone morphogenic proteins share substantial amino acid sequence homology in their C-terminal regions (domains). Typically, the above-mentioned naturally occurring osteogenic proteins are translated as a precursor, having an N-terminal signal peptide sequence typically less than about 30 residues, followed by a “pro” domain that is cleaved to yield the mature C-terminal domain of approximately 100-140 amino acids. The signal peptide is cleaved rapidly upon translation, at a cleavage site that can be predicted in a given sequence using the method of Von Heijne, Nucleic Acids Research, 14, pp. 4683-4691 (1986). The pro domain typically is about three times larger than the fully processed mature C-terminal domain.

Another characteristic of the BMP protein family members is their ability to dimerize. Several bone-deprived osteogenic proteins (OPs) and BMPs are found as homo- and heterodimers in their active forms. The ability of OPs and BMPs to form heterodimers may confer additional or altered morphogenic inductive capabilities on morphogenic proteins. Heterodimers may exhibit qualitatively or quantitatively different binding affinities than homodimers for OP and BMP receptor molecules. Altered binding affinities may in turn lead to differential activation of receptors that mediate different signaling pathways, which may ultimately lead to different biological activities or outcomes. Altered binding affinities could also be manifested in a tissue or cell type-specific manner, thereby inducing only particular progenitor cell types to undergo proliferation and/or differentiation.

In preferred embodiments, the pair of morphogenic polypeptides have amino acid sequences each comprising a sequence that shares a defined relationship with an amino acid sequence of a reference morphogen. Herein, preferred osteogenic polypeptides share a defined relationship with a sequence present in osteogenically active human OP-1, SEQ ID NO: 1. However, any one or more of the naturally occurring or biosynthetic sequences disclosed herein similarly could be used as a reference sequence. Preferred osteogenic polypeptides share a defined relationship with at least the C-terminal six cysteine domain of human OP-1, residues 335-431 of SEQ ID NO: 1. Preferably, osteogenic polypeptides share a defined relationship with at least the C-terminal seven cysteine domain of human OP-1, residues 330-431 of SEQ ID NO: 1. That is, preferred polypeptides in a dimeric protein with bone morphogenic activity each comprise a sequence that corresponds to a reference sequence or is functionally equivalent thereto.

Functionally equivalent sequences include functionally equivalent arrangements of cysteine residues disposed within the reference sequence, including amino acid insertions or deletions which alter the linear arrangement of these cysteines, but do not materially impair their relationship in the folded structure of the dimeric morphogen protein, including their ability to form such intra- or inter-chain disulfide bonds as may be necessary for morphogenic activity. Functionally equivalent sequences further include those wherein one or more amino acid residues differs from the corresponding residue of a reference sequence, e.g., the C-terminal seven cysteine domain (also referred to herein as the conserved seven cysteine skeleton) of human OP-1, provided that this difference does not destroy bone morphogenic activity. Accordingly, conservative substitutions of corresponding amino acids in the reference sequence are preferred. Particularly preferred conservative substitutions are those fulfilling the criteria defined for an accepted point mutation in Dayhoff et al., supra, the teachings of which are incorporated by reference herein.

The osteogenic protein OP-1 has been described (see, e.g., Oppermann et al., U.S. Pat. No. 5,354,557, incorporated herein by reference). Natural-sourced osteogenic protein in its mature, native form is a glycosylated dimer typically having an apparent molecular weight of about 30-36 kDa as determined by SDS-PAGE. When reduced, the 30 kDa protein gives rise to two glycosylated peptide subunits having apparent molecular weights of about 16 kDa and 18 kDa. The unglycosylated protein, which also has osteogenic activity, has an apparent molecular weight of about 27 kDa. When reduced, the 27 kDa protein gives rise to two unglycosylated polypeptides, having molecular weights of about 14 kDa to 16 kDa, capable of inducing endochondral bone formation in a mammal. Osteogenic proteins may include forms having varying glycosylation patterns, varying N-termini, and active truncated or mutated forms of native protein.

As described above, particularly useful sequences include those comprising the C-terminal 96 or 102 amino acid sequences of DPP (from Drosophila), Vg1 (from Xenopus), Vgr-1 (from mouse), the OP-1 and OP-2 proteins,(see U.S. Pat. No. 5,011,691 and Oppermann et al., incorporated herein by reference), as well as the proteins referred to as BMP-2, BMP-3, BMP-4 (see WO 88/00205, U.S. Pat. No. 5,013,649 and WO 91/18098, incorporated herein by reference), BMP-5 and BMP-6 (see WO 90/11366, PCT/US90/01630, incorporated herein by reference), BMP-8 and BMP-9.

Preferred first and second morphogenic and osteogenic proteins of this invention independently comprise at least one polypeptide selected from the group consisting of OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3 (BMP-12), CDMP-2 (BMP-13), CDMP-1 (BMP-14), BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, NEURAL and amino acid sequence variants and homologs thereof, including species homologs thereof and fragments thereof. In some embodiments, the first morphogenic protein is OP-1 (BMP-7) or a fragment thereof, and the second morphogenic protein is selected from the group consisting of CDMP-1, CDMP-2, and CDMP3 and fragments thereof. In some embodiments, the second morphogenic protein is CDMP-1 or a fragment thereof. In some embodiments, the second morphogenic protein is CDMP-2 or a fragment thereof. In some embodiments, the second morphogenic protein is CDMP-3 or a fragment thereof.

A second morphogenic protein according to this invention is a factor that is capable of stimulating the ability of a first morphogenic protein to induce tissue formation from a progenitor cell. In one embodiment of this invention, a method for improving the tissue inductive activity of a first morphogenic protein in a mammal by coadministering an effective amount of a second morphogenic protein is provided. In some embodiments, nucleic acids encoding the first and second morphogenic proteins are administered. In other embodiments, vectors comprising nucleic acids encoding the first and second morphogenic proteins operably linked to an expression control sequence are administered. In yet other embodiments, cells comprising vectors comprising nucleic acids encoding the first and second morphogenic proteins operably linked to an expression control sequence are administered. Preferably, the second morphogenic protein has a synergistic effect on the tissue induction by the first morphogenic protein.

The progenitor cell that is induced to proliferate and/or differentiate by the combination of morphogenic proteins of this invention is preferably a mammalian cell. Preferred progenitor cells include mammalian chondroblasts, osteoblasts and neuroblasts, all earlier developmental precursors thereof, and all cells that develop therefrom (e.g., chondroblasts, pre-chondroblasts and chondrocytes). However, morphogenic proteins are highly conserved throughout evolution, and non-mammalian progenitor cells are also likely to be stimulated by same- or cross-species first morphogenic proteins and a second morphogenic protein combinations. It is, thus, envisioned that when schemes become available for implanting xenogeneic cells into humans without causing adverse immunological reactions, non-mammalian progenitor cells stimulated by a first morphogenic protein and a second morphogenic protein according to the procedures set forth herein will be useful for tissue regeneration and repair in humans.

A second morphogenic protein selected for use in concert with a first morphogenic proteins according to the desired tissue type to be induced and the site at which the first morphogenic protein and second morphogenic protein will be administered. The particular choice of a first morphogenic protein/second morphogenic protein combination and the relative concentrations at which they are combined may be varied systematically to optimize the tissue type induced at a selected treatment site using the procedures described herein.

Preferably, the second morphogenic protein is present in an amount capable of synergistically stimulating the tissue inductive activity of the first morphogenic protein in a mammal. The relative concentrations of the first morphogenic protein and the second morphogenic protein that will optimally induce tissue formation when administered to a mammal may be determined empirically by the skilled practitioner using the procedures described herein.

Publications disclosing these sequences, as well as their chemical and physical properties, include: OP-1 and OP-2 (U.S. Pat. No. 5,011,691; U.S. Pat. No. 5,266,683; Ozkaynak et al., EMBO J., 9, pp. 2085-2093 (1990); OP-3 (WO 94/10203 (PCT US93/10520)), BMP-2, BMP-3, BMP-4, (WO 88/00205; Wozney et al. Science, 242, pp. 1528-1534 (1988)), BMP-5 and BMP-6, (Celeste et al., PNAS, 87, 9843-9847 (1991)), Vgr-1 (Lyons et al., PNAS, 86, pp. 4554-4558 (1989)); DPP (Padgett et al. Nature, 325, pp. 81-84 (1987)); Vg-1 (Weeks, Cell, 51, pp. 861-867 (1987)); BMP-9 (W095/33830 (PCT/US95/07084); BMP-10 (WO 94/26893 (PCT/US94/05290); BMP-11 (WO 94/26892 (PCT/US94/05288); BMP-12 (WO95/16035 (PCT/US94/14030); BMP-13 (WO95/16035 (PCT/US94/14030); GDF-1 (WO 92/00382 (PCT/US91/04096) and Lee et al. PNAS, 88, pp. 4250-4254 (1991); GDF-8 (WO 94/21681 (PCT/US94/03019); GDF-9 (WO 94/15966 (PCT/US94/00685); GDF-10 (WO 95/10539 (PCT/US94/11440); GDF-11 (WO 96/01845 (PCT/US95/08543); BMP-15 (WO 96/36710 (PCT/US96/06540); MP-121 (WO 96/01316 (PCT/EP95/02552); GDF-5 (CDMP-1, MP52) (WO 94/15949 (PCT/US94/00657) and WO 96/14335 (PCT/US94/12814) and WO 93/16099 (PCT/EP93/00350)); GDF-6 (CDMP-2, BMP13) (WO 95/01801 (PCT/US94/07762) and WO 96/14335 and WO 95/10635 (PCT/US94/14030)); GDF-7 (CDMP-3, BMP12) (WO 95/10802 (PCT/US94/07799) and WO 95/10635 (PCT/US94/14030)). The above publications are incorporated herein by reference.

In another embodiment, useful proteins include biologically active biosynthetic constructs, including novel biosynthetic morphogenic proteins and chimeric proteins designed using sequences from two or more known morphogens.

In another embodiment of this invention, a first or second morphogenic protein may be prepared synthetically to induce tissue formation. Morphogenic proteins prepared synthetically may be native, or may be non-native proteins, i.e., those not otherwise found in nature. Non-native osteogenic proteins have been synthesized using a series of consensus DNA sequences (U.S. Pat. No. 5,324,819, incorporated herein by reference). These consensus sequences were designed based on partial amino acid sequence data obtained from natural osteogenic products and on their observed homologies with other genes reported in the literature having a presumed or demonstrated developmental function.

Several of the biosynthetic consensus sequences (called consensus osteogenic proteins or “COPs”) have been expressed as fusion proteins in prokaryotes. Purified fusion proteins may be cleaved, refolded, implanted in an established animal model and shown to have bone- and/or cartilage-inducing activity. The currently preferred synthetic osteogenic proteins comprise two synthetic amino acid sequences designated COP-5 (SEQ. ID NO: 2) and COP-7 (SEQ. ID NO: 3).

Oppermann et al., U.S. Pat. Nos. 5,011,691 and 5,324,819, which are incorporated herein by reference, describe the amino acid sequences of COP-5 and COP-7 as shown below:

| | |
|---|---|
| COP5 | LYVDFS-DVGWDDWIVAPPGYQAFYCHGECPFPLAD |
| COP7 | LYVDFS-DVGWNDWIVAPPGYHAFYCHGECPFPLAD |
| COP5 | HFNSTN--H-AVVQTLVNSVNSKI--PKACCVPTELSA |
| COP7 | HLNSTN--H-AVVQTLVNSVNSKI--PKACCVPTELSA |
| COP5 | ISMLYLDENEKVVLKYNQEMVVEGCGCR |
| COP7 | ISMLYLDENEKVVLKYNQEMVVEGCGCR |

In these amino acid sequences, the dashes (-) are used as fillers only to line up comparable sequences in related proteins. Differences between the aligned amino acid sequences are highlighted.

The DNA and amino acid sequences of these and other BMP family members are published and may be used by those of skill in the art to determine whether a newly identified protein belongs to the BMP family. New BMP-related gene products are expected by analogy to possess at least one morphogenic activity and thus classified as a BMP. In some embodiments, useful proteins include fragments of morphogenic proteins. These fragments may be synthetic peptides or portions of naturally occurring proteins.

In one preferred embodiment of this invention, the first and second morphogenic proteins independently comprise a pair of subunits disulfide bonded to produce a dimeric species, wherein at least one of the subunits comprises a recombinant peptide belonging to the BMP protein family. In another preferred embodiment of this invention, the first and second morphogenic proteins independently comprise a pair of subunits that produce a dimeric species formed through non-covalent interactions, wherein at least one of the subunits comprises a recombinant peptide belonging to the BMP protein family. Non-covalent interactions include Van der Waals, hydrogen bond, hydrophobic and electrostatic interactions. The dimeric species may be a homodimer or heterodimer and is capable of inducing cell proliferation and/or tissue formation. In some embodiments, the morphogenic proteins are capable of inducing cell proliferation and/or tissue formation when accessible to a progenitor cell in the mammal the progenitor may be induced to form one or more tissue types preferably selected from endochondral or intramembranous bone, cartilage, tendon and ligament tissue. In some embodiments, the first and second morphogenic proteins are each independently monomers.

In certain preferred embodiments, the first and second bone morphogenic proteins useful herein independently include those in which the amino acid sequences comprise a sequence sharing at least 70% amino acid sequence homology or “similarity”, preferably 80%, more preferably 90%, even more preferably 95%, even more preferably 98% homology or similarity, with a reference morphogenic protein selected from the foregoing naturally occurring proteins. Preferably, the reference protein is human OP-1, and the reference sequence thereof is the C-terminal seven cysteine domain present in osteogenically active forms of human OP-1, residues 330-431 of SEQ ID NO: 1. In some embodiments, at least one of the first and second morphogenic proteins comprises a dimeric protein having an amino acid sequence having at least 70% homology within the C-terminal 102-106 amino acids of human OP-1. In certain embodiments, a polypeptide suspected of being functionally equivalent to a reference morphogenic polypeptide is aligned therewith using the method of Needleman, et al., supra, implemented conveniently by computer programs such as the Align program (DNAstar, Inc.). As noted above, internal gaps and amino acid insertions in the candidate sequence are ignored for purposes of calculating the defined relationship, conventionally expressed as a level of amino acid sequence homology or identity, between the candidate and reference sequences. In one preferred embodiment, the reference sequence is OP-1. In another preferred embodiment, the reference sequence is selected from CDMP-1, CDMP-2 or CDMP-3. Bone morphogenic proteins useful herein accordingly include allelic, phylogenetic counterpart and other variants of the preferred reference sequence, whether naturally-occurring or biosynthetically produced (e.g., including “muteins” or “mutant proteins”), as well as novel members of the general morphogenic family of proteins, including those set forth and identified above. Certain particularly preferred morphogenic polypeptides share at least 60% amino acid identity with the preferred reference sequence of human OP-1, still more preferably at least 65% amino acid identity therewith.

In another embodiment, useful osteogenic proteins include those sharing the conserved seven cysteine domain and sharing at least 70% amino acid sequence homology (similarity) within the C-terminal active domain, as defined herein. In still another embodiment, the osteogenic proteins of the invention can be defined as osteogenically active proteins having any one of the generic sequences defined herein, including OPX (SEQ ID NO: 4) and Generic Sequences 7 (SEQ ID NO: 5) and 8 (SEQ ID NO: 6), or Generic Sequences 9 (SEQ ID NO: 7) and 10 (SEQ ID NO: 8).

The family of bone morphogenic polypeptides useful in the present invention, and members thereof, can be defined by a generic amino acid sequence. For example, Generic Sequence 7 (SEQ ID NO: 5) and Generic Sequence 8 (SEQ ID NO: 6) are 97 and 102 amino acid sequences, respectively, and accommodate the homologies shared among preferred protein family members identified to date, including at least OP-1, OP-2, OP-3, CBMP-2A, CBMP-2B, BMP-3, 60A, DPP, Vg1, BMP-5, BMP-6, Vgr-1, and GDF-1. The amino acid sequences for these proteins are described herein and/or in the art, as summarized above. The generic sequences include both the amino acid identity shared by these sequences in the C-terminal domain, defined by the six and seven cysteine skeletons (Generic Sequences 7 and 8, respectively), as well as alternative residues for the variable positions within the sequence. The generic sequences provide an appropriate cysteine skeleton where inter- or intramolecular disulfide bonds can form, and contain certain critical amino acids likely to influence the tertiary structure of the folded proteins. In addition, the generic sequences allow for an additional cysteine at position 36 (Generic Sequence 7) or position 41 (Generic Sequence 8), thereby encompassing the morphogenically active sequences of OP-2 and OP-3.

```
Generic Sequence 7
Leu Xaa Xaa Xaa Phe Xaa Xaa Xaa Gly Trp Xaa Xaa
1 5 10
Xaa Xaa Xaa Xaa Pro Xaa Xaa Xaa Xaa Ala Xaa Tyr
15 20
Cys Xaa Gly Xaa Cys Xaa Xaa Pro Xaa Xaa Xaa Xaa
25 30 35
Xaa Xaa Xaa Xaa Asn His Ala Xaa Xaa Xaa Xaa Xaa
                        40 45
Xaa Xaa Xaa Xaa Xaa Xaa Xaa Xaa Xaa Xaa Xaa Xaa
        50 55 60
Cys Cys Xaa Pro Xaa Xaa Xaa Xaa Xaa Xaa Xaa Xaa
                                65 70
Leu Xaa Xaa Xaa Xaa Xaa Xaa Xaa Val Xaa Leu Xaa
                75 80
Xaa Xaa Xaa Xaa Met Xaa Val Xaa Xaa Cys Xaa Cys
85 90 95
Xaa
```

wherein each Xaa independently is selected from a group of one or more specified amino acids defined as follows: “res.” means “residue” and Xaa at res.2=(Tyr or Lys); Xaa at res.3=Val or Ile); Xaa at res.4=(Ser, Asp or Glu); Xaa at res.6=(Arg, Gln, Ser, Lys or Ala); Xaa at res.7=(Asp or Glu); Xaa at res.8=(Leu, Val or Ile); Xaa at res. 11=(Gln, Leu, Asp, His, Asn or Ser); Xaa at res.12=(Asp, Arg, Asn or Glu); Xaa at res.13=(Trp or Ser); Xaa at res.14=(Ile or Val); Xaa at res.15=(Ile or Val); Xaa at res.16 (Ala or Ser); Xaa at res.18=(Glu, Gln, Leu, Lys, Pro or Arg); Xaa at res.19=(Gly or Ser); Xaa at res.20=(Tyr or Phe); Xaa at res.21=(Ala, Ser, Asp, Met, His, Gln, Leu or Gly); Xaa at res.23=(Tyr, Asn or Phe); Xaa at res.26=(Glu, His, Tyr, Asp, Gln, Ala or Ser); Xaa at res.28=(Glu, Lys, Asp, Gln or Ala); Xaa at res.30=(Ala, Ser, Pro, Gln, Ile or Asn); Xaa at res.31=(Phe, Leu or Tyr); Xaa at res.33=(Leu, Val or Met); Xaa at res.34=(Asn, Asp, Ala, Thr or Pro); Xaa at res.35=(Ser, Asp, Glu, Leu, Ala or Lys); Xaa at res.36=(Tyr, Cys, His, Ser or Ile); Xaa at res.37=(Met, Phe, Gly or Leu); Xaa at res.38=(Asn, Ser or Lys); Xaa at res.39=(Ala, Ser, Gly or Pro); Xaa at res.40=(Thr, Leu or Ser); Xaa at res.44=(Ile, Val or Thr); Xaa at res.45=(Val, Leu, Met or Ile); Xaa at res.46=(Gln or Arg); Xaa at res.47=(Thr, Ala or Ser); Xaa at res.48=(Leu or Ile); Xaa at res.49=(Val or Met); Xaa at res.50=(His, Asn or Arg); Xaa at res.51=(Phe, Leu, Asn, Ser, Ala or Val); Xaa at res.52=(Ile, Met, Asn, Ala, Val, Gly or Leu); Xaa at res.53=(Asn, Lys, Ala, Glu, Gly or Phe); Xaa at res.54=(Pro, Ser or Val); Xaa at res.55=(Glu, Asp, Asn, Gly, Val, Pro or Lys); Xaa at res.56=(Thr, Ala, Val, Lys, Asp, Tyr, Ser, Gly, Ile or His); Xaa at res.57=(Val, Ala or Ile); Xaa at res.58=(Pro or Asp); Xaa at res.59=(Lys, Leu or Glu); Xaa at res.60=(Pro, Val or Ala); Xaa at res.63=(Ala or Val); Xaa at res.65=(Thr, Ala or Glu); Xaa at res.66=(Gln, Lys, Arg or Glu); Xaa at res.67=(Leu, Met or Val); Xaa at res.68=(Asn, Ser, Asp or Gly); Xaa at res.69=(Ala, Pro or Ser); Xaa at res.70=(Ile, Thr, Val or Leu); Xaa at res.71=(Ser, Ala or Pro); Xaa at res.72=(Val, Leu, Met or Ile); Xaa at res.74=(Tyr or Phe); Xaa at res.75=(Phe, Tyr, Leu or His); Xaa at res.76=(Asp, Asn or Leu); Xaa at res.77=(Asp, Glu, Asn, Arg or Ser); Xaa at res.78=(Ser, Gln, Asn, Tyr or Asp); Xaa at res.79=(Ser, Asn, Asp, Glu or Lys); Xaa at res.80=(Asn, Thr or Lys); Xaa at res.82=(Ile, Val or Asn); Xaa at res.84=(Lys or Arg); Xaa at res.85=(Lys, Asn, Gln, His, Arg or Val); Xaa at res.86=(Tyr, Glu or His); Xaa at res.87=(Arg, Gln, Glu or Pro); Xaa at res.88=(Asn, Glu, Trp or Asp); Xaa at res.90=(Val, Thr, Ala or Ile); Xaa at res.92=(Arg, Lys, Val, Asp, Gln or Glu); Xaa at res.93=(Ala, Gly, Glu or Ser); Xaa at res.95=(Gly or Ala) and Xaa at res.97=(His or Arg).

Generic Sequence 8 (SEQ ID NO: 6) includes all of Generic Sequence 7 and in addition includes the following sequence (SEQ ID NO: 9) at its N-terminus:

SEQ ID NO: 9
Cys Xaa Xaa Xaa Xaa
1 5

Accordingly, beginning with residue 7, each “Xaa” in Generic Sequence 8 is a specified amino acid defined as for Generic Sequence 7, with the distinction that each residue number described for Generic Sequence 7 is shifted by five in Generic Sequence 8. Thus, “Xaa at res.2=(Tyr or Lys)” in Generic Sequence 7 refers to Xaa at res.7 in Generic Sequence 8. In Generic Sequence 8, Xaa at res.2=(Lys, Arg, Ala or Gln); Xaa at res.3=(Lys, Arg or Met); Xaa at res.4=(His, Arg or Gln); and Xaa at res.5=(Glu, Ser, His, Gly, Arg, Pro, Thr, or Tyr).

In another embodiment, useful osteogenic proteins include those defined by Generic Sequences 9 and 10, defined as follows.

Specifically, Generic Sequences 9 and 10 are composite amino acid sequences of the following proteins: human OP-1, human OP-2, human OP-3, human BMP-2, human BMP-3, human BMP-4, human BMP-5, human BMP-6, human BMP-8, human BMP-9, human BMP-10, human BMP-11, Drosophila 60A, Xenopus Vg-1, sea urchin UNIVIN, human CDMP-1 (mouse GDF-5), human CDMP-2 (mouse GDF-6, human BMP-13), human CDMP-3 (mouse GDF-7, human BMP-12), mouse GDF-3, human GDF-1, mouse GDF-1, chicken DORSALIN, dpp, Drosophila SCREW, mouse NODAL, mouse GDF-8, human GDF-8, mouse GDF-9, mouse GDF-10, human GDF-11, mouse GDF-11, human BMP-15, and rat BMP3b. Like Generic Sequence 7, Generic Sequence 9 is a 97 amino acid sequence that accommodates the C-terminal six cysteine skeleton and, like Generic Sequence 8, Generic Sequence 10 is a 102 amino acid sequence which accommodates the seven cysteine skeleton.

```
Generic Sequence 9
Xaa Xaa Xaa Xaa Xaa Xaa Xaa Xaa Xaa Xaa Xaa Xaa
1 5 10
Xaa Xaa Xaa Xaa Pro Xaa Xaa Xaa Xaa Xaa Xaa Xaa
                15 20
Cys Xaa Gly Xaa Cys Xaa Xaa Xaa Xaa Xaa Xaa Xaa
25 30 35
Xaa Xaa Xaa Xaa Xaa Xaa Xaa Xaa Xaa Xaa Xaa Xaa
                        40 45
Xaa Xaa Xaa Xaa Xaa Xaa Xaa Xaa Xaa Xaa Xaa Xaa
        50 55 60
Xaa Cys Xaa Pro Xaa Xaa Xaa Xaa Xaa Xaa Xaa Xaa
                                65 70
Leu Xaa Xaa Xaa Xaa Xaa Xaa Xaa Xaa Xaa Xaa Xaa
                75 80
Xaa Xaa Xaa Xaa Xaa Xaa Xaa Xaa Xaa Cys Xaa Cys
85 90 95
Xaa
```

wherein each Xaa is indendently selected from a group of one or more specified amino acids defined as follows: “res.” means “residue” and Xaa at res.1=(Phe, Leu or Glu); Xaa at res.2=(Tyr, Phe, His, Arg, Thr, Lys, Gln, Val or Glu); Xaa at res.3=(Val, Ile, Leu or Asp); Xaa at res.4=(Ser, Asp, Glu, Asn or Phe); Xaa at res.5=(Phe or Glu); Xaa at res.6=(Arg, Gln, Lys, Ser, Glu, Ala or Asn); Xaa at res.7=(Asp, Glu, Leu, Ala or Gln); Xaa at res.8=(Leu, Val, Met, Ile or Phe); Xaa at res.9=(Gly, His or Lys); Xaa at res.10=(Trp or Met); Xaa at res.11=(Gln, Leu, His, Glu, Asp, Ser or Gly); Xaa at res.12=(Asp, Asn, Ser, Lys, Arg, Glu or His); Xaa at res.13=(Trp or Ser); Xaa at res.14=(Ile or Val); Xaa at res.15=(Ile or Val); Xaa at res.16=(Ala, Ser, Tyr or Trp); Xaa at res. 18=(Gly, Lys, Gln, Met, Pro, Leu, Arg, His or Lys); Xaa at res.19=(Gly, Glu, Asp, Lys, Ser, Gln, Arg or Phe); Xaa at res.20=(Tyr or Phe); Xaa at res.21=(Ala, Ser, Gly, Met, Gln, His, Glu, Asp, Leu, Asn, Lys or Thr); Xaa at res.22=(Ala or Pro); Xaa at res.23=(Tyr, Phe, Asn, Ala or Arg); Xaa at res.24=(Tyr, His, Glu, Phe or Arg); Xaa at res.26=(Glu, Asp, Ala, Ser, Tyr, His, Lys, Arg, Gln or Gly); Xaa at res.28=(Glu, Asp, Leu, Val, Lys, Gly, Thr, Ala or Gln); Xaa at res.30=(Ala, Ser, Ile, Asn, Pro, Glu, Asp, Phe, Gln or Leu); Xaa at res.31=(Phe, Tyr, Leu, Asn, Gly or Arg); Xaa at res.32=(Pro, Ser, Ala or Val); Xaa at res.33=(Leu, Met, Glu, Phe or Val); Xaa at res.34=(Asn, Asp, Thr, Gly, Ala, Arg, Leu or Pro); Xaa at res.35=(Ser, Ala, Glu, Asp, Thr, Leu, Lys, Gln or His); Xaa at res.36=(Tyr, His, Cys, Ile, Arg, Asp, Asn, Lys, Ser, Glu or Gly); Xaa at res.37=(Met, Leu, Phe, Val, Gly or Tyr); Xaa at res.38=(Asn, Glu, Thr, Pro, Lys, His, Gly, Met, Val or Arg); Xaa at res.39=(Ala, Ser, Gly, Pro or Phe); Xaa at res.40=(Thr, Ser, Leu, Pro, His or Met); Xaa at res.41=(Asn, Lys, Val, Thr or Gln); Xaa at res.42=(His, Tyr or Lys); Xaa at res.43=(Ala, Thr, Leu or Tyr); Xaa at res.44=(Ile, Thr, Val, Phe, Tyr, Met or Pro); Xaa at res.45=(Val, Leu, Met, Ile or His); Xaa at res.46=(Gln, Arg or Thr); Xaa at res.47=(Thr, Ser, Ala, Asn or His); Xaa at res.48=(Leu, Asn or Ile); Xaa at res.49=(Val, Met, Leu, Pro or Ile); Xaa at res.50=(His, Asn, Arg, Lys, Tyr or Gln); Xaa at res.51=(Phe, Leu, Ser, Asn, Met, Ala, Arg, Glu, Gly or Gln); Xaa at res.52=(Ile, Met, Leu, Val, Lys, Gln, Ala or Tyr); Xaa at res.53=(Asn, Phe, Lys, Glu, Asp, Ala, Gln, Gly, Leu or Val); Xaa at res.54=(Pro, Asn, Ser, Val or Asp); Xaa at res.55=(Glu, Asp, Asn, Lys, Arg, Ser, Gly, Thr, Gln, Pro or His); Xaa at res.56=(Thr, His, Tyr, Ala, Ile, Lys, Asp, Ser, Gly or Arg); Xaa at res.57=(Val, Ile, Thr, Ala, Leu or Ser); Xaa at res.58=(Pro, Gly, Ser, Asp or Ala); Xaa at res.59=(Lys, Leu, Pro, Ala, Ser, Glu, Arg or Gly); Xaa at res.60=(Pro, Ala, Val, Thr or Ser); Xaa at res.61=(Cys, Val or Ser); Xaa at res.63=(Ala, Val or Thr); Xaa at res.65=(Thr, Ala, Glu, Val, Gly, Asp or Tyr); Xaa at res.66=(Gln, Lys, Glu, Arg or Val); Xaa at res.67=(Leu, Met, Thr or Tyr); Xaa at res.68=(Asn, Ser, Gly, Thr, Asp, Glu, Lys or Val); Xaa at res.69=(Ala, Pro, Gly or Ser); Xaa at res.70=(Ile, Thr, Leu or Val); Xaa at res.71=(Ser, Pro, Ala, Thr, Asn or Gly); Xaa at res.72=(Val, Ile, Leu or Met); Xaa at res.74=(Tyr, Phe, Arg, Thr, Tyr or Met); Xaa at res.75=(Phe, Tyr, His, Leu, Ile, Lys, Gln or Val); Xaa at res.76=(Asp, Leu, Asn or Glu); Xaa at res.77=(Asp, Ser, Arg, Asn, Glu, Ala, Lys, Gly or Pro); Xaa at res.78=(Ser, Asn, Asp, Tyr, Ala, Gly, Gln, Met, Glu, Asn or Lys); Xaa at res.79=(Ser, Asn, Glu, Asp, Val, Lys, Gly, Gln or Arg); Xaa at res.80=(Asn, Lys, Thr, Pro, Val, Ile, Arg, Ser or Gln); Xaa at res.81=(Val, Ile, Thr or Ala); Xaa at res.82=(Ile, Asn, Val, Leu, Tyr, Asp or Ala); Xaa at res.83=(Leu, Tyr, Lys or Ile); Xaa at res.84=(Lys, Arg, Asn, Tyr, Phe, Thr, Glu or Gly); Xaa at res.85=(Lys, Arg, His, Gln, Asn, Glu or Val); Xaa at res.86=(Tyr, His, Glu or Ile); Xaa at res.87=(Arg, Glu, Gln, Pro or Lys); Xaa at res.88=(Asn, Asp, Ala, Glu, Gly or Lys); Xaa at res.89=(Met or Ala); Xaa at res.90=(Val, Ile, Ala, Thr, Ser or Lys); Xaa at res.91=(Val or Ala); Xaa at res.92=(Arg, Lys, Gln, Asp, Glu, Val, Ala, Ser or Thr); Xaa at res.93=(Ala, Ser, Glu, Gly, Arg or Thr); Xaa at res.95=(Gly, Ala or Thr); Xaa at res.97=(His, Arg, Gly, Leu or Ser). Further, after res.53 in rBMP3b and mGDF-10 there is an Ile; after res.54 in GDF-1 there is a T; after res.54 in BMP3 there is a V; after res.78 in BMP-8 and Dorsalin there is a G; after res.37 in hGDF-1 there is Pro, Gly, Gly, Pro.

Generic Sequence 10 (SEQ ID NO: 8) includes all of Generic Sequence 9 (SEQ ID NO: 7) and in addition includes the following sequence (SEQ ID NO: 9) at its N-terminus:

SEQ ID NO: 9
Cys Xaa Xaa Xaa Xaa
1 5

Accordingly, beginning with residue 6, each “Xaa” in Generic Sequence 10 is a specified amino acid defined as for Generic Sequence 9, with the distinction that each residue number described for Generic Sequence 9 is shifted by five in Generic Sequence 10. Thus, “Xaa at res.1=(Tyr, Phe, His, Arg, Thr, Lys, Gln, Val or Glu)” in Generic Sequence 9 refers to Xaa at res.6 in Generic Sequence 10. In Generic Sequence 10, Xaa at res.2=(Lys, Arg, Gln, Ser, His, Glu, Ala, or Cys); Xaa at res.3=(Lys, Arg, Met, Lys, Thr, Leu, Tyr, or Ala); Xaa at res.4=(His, Gin, Arg, Lys, Thr, Leu, Val, Pro, or Tyr); and Xaa at res.5=(Gln, Thr, His, Arg, Pro, Ser, Ala, Gln, Asn, Tyr, Lys, Asp, or Leu).

As noted above, certain currently preferred bone morphogenic polypeptide sequences useful in this invention have greater than 60% identity, preferably greater than 65% identity, with the amino acid sequence defining the preferred reference sequence of hOP-1. These particularly preferred sequences include allelic and phylogenetic counterpart variants of the OP-1 and OP-2 proteins, including the Drosophila 60A protein. Accordingly, in certain particularly preferred embodiments, useful morphogenic proteins include active proteins comprising pairs of polypeptide chains within the generic amino acid sequence herein referred to as “OPX” (SEQ ID NO: 4), which defines the seven cysteine skeleton and accommodates the homologies between several identified variants of OP-1 and OP-2. As described therein, each Xaa at a given position independently is selected from the residues occurring at the corresponding position in the C-terminal sequence of mouse or human OP-1 or OP-2.

```
SEQ ID NO: 4
Cys Xaa Xaa His Glu Leu Tyr Val Ser Phe Xaa Asp
1 5 10
Leu Gly Trp Xaa Asp Trp Xaa Ile Ala Pro Xaa Gly
                15 20
Tyr Xaa Ala Tyr Tyr Cys Glu Gly Glu Cys Xaa Phe
25 30 35
Pro Leu Xaa Ser Xaa Met Asn Ala Thr Asn His Ala
                        40 45
Ile Xaa Gln Xaa Leu Val His Xaa Xaa Xaa Pro Xaa
        50 55 60
Xaa Val Pro Lys Xaa Cys Cys Ala Pro Thr Xaa Leu
                                65 70
Xaa Ala Xaa Ser Val Leu Tyr Xaa Asp Xaa Ser Xaa
                75 80
Asn Val Ile Leu Xaa Lys Xaa Arg Asn Met Val Val
85 90 95
Xaa Ala Cys Gly Cys His
                        100
```

wherein Xaa at res.2=(Lys or Arg); Xaa at res.3=(Lys or Arg); Xaa at res.11=(Arg or Gln); Xaa at res.16=(Gln or Leu); Xaa at res.19=(Ile or Val); Xaa at res.23=(Glu or Gln); Xaa at res.26=(Ala or Ser); Xaa at res.35=(Ala or Ser); Xaa at res.39=(Asn or Asp); Xaa at res.41=(Tyr or Cys); Xaa at res.50=(Val or Leu); Xaa at res.52=(Ser or Thr); Xaa at res.56=(Phe or Leu); Xaa at res.57=(Ile or Met); Xaa at res.58=(Asn or Lys); Xaa at res.60=(Glu, Asp or Asn); Xaa at res.61=(Thr, Ala or Val); Xaa at res.65=(Pro or Ala); Xaa at res.71=(Gln or Lys); Xaa at res.73=(Asn or Ser); Xaa at res.75=(Ile or Thr); Xaa at res.80=(Phe or Tyr); Xaa at res.82=(Asp or Ser); Xaa at res.84=(Ser or Asn); Xaa at res.89=(Lys or Arg); Xaa at res.91=(Tyr or His); and Xaa at res.97=(Arg or Lys).

In still another preferred embodiment, useful osteogenically active proteins have polypeptide chains with amino acid sequences comprising a sequence encoded by a nucleic acid that hybridizes, under low, medium or high stringency hybridization conditions, to DNA or RNA encoding reference morphogen sequences, e.g., C-terminal sequences defining the conserved seven cysteine domains of OP-1, OP-2, BMP-2, BMP-4, BMP-5, BMP-6, 60A, GDF-3, GDF-6, GDF-7 and the like. As used herein, high stringent hybridization conditions are defined as hybridization according to known techniques in 40% formamide, 5×SSPE, 5× Denhardt's Solution, and 0.1% SDS at 37° C. overnight, and washing in 0.1×SSPE, 0.1% SDS at 50° C. Standard stringent conditions are well characterized in commercially available, standard molecular cloning texts. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984): Nucleic Acid Hybridization (B. D. Hames & S. J. Higgins eds. 1984); and B. Perbal, A Practical Guide To Molecular Cloning (1984), the disclosures of which are incorporated herein by reference.

As noted above, proteins useful in the present invention generally are dimeric proteins comprising a folded pair of the above polypeptides. In some embodiments, the pair of polypeptides are not disulfide bonded. In some embodiments the pair of polypeptides are disulfide bonded. Such disulfide bonded morphogenic proteins are inactive when reduced, but are active as oxidized homodimers and when oxidized in combination with others of this invention to produce heterodimers. Thus, members of a folded pair of morphogenic polypeptides in a morphogenically active protein can be selected independently from any of the specific polypeptides mentioned above.

The first and second bone morphogenic proteins useful in the materials and methods of this invention include proteins comprising any of the polypeptide chains described above, whether isolated from naturally-occurring sources, or produced by recombinant DNA or other synthetic techniques, and includes allelic and phylogenetic counterpart variants of these proteins, as well as muteins thereof, and various truncated and fusion constructs. Deletion or addition mutants also are envisioned to be active, including those which may alter the conserved C-terminal six or seven cysteine domain, provided that the alteration does not functionally disrupt the relationship of these cysteines in the folded structure. Accordingly, such active forms are considered the equivalent of the specifically described constructs disclosed herein. The proteins may include forms having varying glycosylation patterns, varying N-termini, a family of related proteins having regions of amino acid sequence homology, and active truncated or mutated forms of native or biosynthetic proteins, produced by expression of recombinant DNA in host cells.

The first and second bone morphogenic proteins contemplated herein can be expressed from intact or truncated cDNA or from synthetic DNAs in prokaryotic or eukaryotic host cells, and purified, cleaved, refolded, and dimerized to form morphogenically active compositions. Currently preferred host cells include, without limitation, prokaryotes including E. coli or eukaryotes including yeast, or mammalian cells, such as CHO, COS or BSC cells. One of ordinary skill in the art will appreciate that other host cells can be used to advantage. Detailed descriptions of the bone morphogenic proteins useful in the practice of this invention, including how to make, use and test them for osteogenic activity, are disclosed in numerous publications, including U.S. Pat. Nos. 5,266,683 and 5,011,691, the disclosures of which are incorporated by reference herein.

Thus, in view of this disclosure and the knowledge available in the art, skilled genetic engineers can isolate genes from cDNA or genomic libraries of various different biological species, which encode appropriate amino acid sequences, or construct DNAs from oligonucleotides, and then can express them in various types of host cells, including both prokaryotes and eukaryotes, to produce large quantities of active proteins capable of stimulating endochondral bone morphogenesis in a mammal.

It is envisioned that the ability to enhance tissue inductive properties of morphogenic proteins using multiple morphogenic proteins, as set forth herein, will be useful for enhancing new tissue inductive properties of the morphogenic proteins. It is also envisioned that the invention described herein will be useful for stimulating tissue inductive activities of new morphogenic proteins that belong to the BMP protein family as they are identified in the future.

Other proteins useful in this invention include eukaryotic proteins identified as osteogenic proteins (see U.S. Pat. No. 5,011,691, incorporated herein by reference), such as the OP-1, OP-2, OP-3 and CBMP-2 proteins, as well as amino acid sequence-related proteins such as DPP (from Drosophila), Vg1 (from Xenopus), Vgr-1 (from mouse), GDF-1 (from humans, see Lee (1991), PNAS 88:4250-4254), 60A (from Drosophila, see Wharton et al. (1991) PNAS 88:9214-9218), dorsalin-1 (from chick, see Basler et al. (1993) Cell 73:687-702 and GenBank accession number L12032) and GDF-5 (from mouse, see Storm et al. (1994) Nature 368:639-643). Additional useful proteins include biosynthetic morphogenic constructs disclosed in U.S. Pat. No. 5,011,691, e.g., COP-1, 3-5, 7 and 16, as well as other proteins known in the art. Still other proteins include osteogenically active forms of BMP-3b (see Takao, et al., (1996), Biochem. Biophys. Res. Comm. 219: 656-662. BMP-9 (see WO95/33830), BMP-15 (see WO 96/35710), BMP-12 (see WO 95/16035), CDMP-1 (see WO 94/12814), CDMP-2 (see WO 94/12814), BMP-10 (see WO 94/26893), GDF-1 (see WO 92/00382), GDF-10 (see WO 95/10539), GDF-3 (see WO 94/15965) and GDF-7 (WO 95/01802).

Production of Morphogenic Proteins

The morphogenic proteins according to this invention may be derived from a variety of sources. Morphogenic proteins may be isolated from natural sources, or may be produced by expressing an appropriate recombinant DNA molecule in a host cell. In addition, the morphogenic proteins of this invention may be derived synthetically and synthetic morphogenic proteins may optionally be expressed from a recombinant DNA molecule in a host cell.

1. Naturally-derived Morphogenic Proteins

In one embodiment of this invention, the morphogenic proteins are isolated from natural sources and used together to induce tissue formation. Morphogenic proteins may be purified from tissue sources, preferably mammalian tissue sources, using conventional physical and chemical separation techniques well known to those of skill in the art. If a purification protocol is unpublished, as for a newly-identified morphogenic protein for example, conventional protein purification techniques may be performed in combination with morphogenic activity assays following each step to trace the morphogenic activity through a series of purification steps thereby establishing a viable purification scheme. When available, immunological reagents may be used alone or in conjunction with the above techniques to purify morphogenic proteins.

This invention also provides native forms of osteogenic proteins to induce tissue formation. Osteogenic protein may be purified from natural sources according to protocols set forth, for example, in Oppermann et al., U.S. Pat. Nos. 5,324,819 and 5,354,557, which are hereby incorporated by reference (see Example 1).

2. Recombinantly-expressed Morphogenic Proteins

In another embodiment of this invention, the morphogenic proteins are produced by the expression of an appropriate recombinant DNA molecule in a host cell and is used to induce tissue formation. The DNA and amino acid sequences of many BMPs and OPs have been reported, and methods for their recombinant production are published and otherwise known to those of skill in the art. For a general discussion of cloning and recombinant DNA technology, see Ausubel et al., supra; see also Watson et al., Recombinant DNA, 2d ed. 1992 (W.H. Freeman and Co., New York).

The DNA sequences encoding bovine and human BMP-2 (formerly BMP-2A) and BMP-4 (formerly BMP-2B), and processes for recombinantly producing the corresponding proteins are described in U.S. Pat. Nos. 5,011,691; 5,013,649; 5,166,058 and 5,168,050.

The DNA and amino acid sequences of human and bovine BMP-5 and BMP-6, and methods for their recombinant production, are disclosed in U.S. Pat. No. 5,106,748, and U.S. Pat. No. 5,187,076, respectively; see also U.S. Pat. Nos. 5,011,691 and 5,344,654. Oppermann et al., U.S. Pat. Nos. 5,011,691 and 5,258,494, disclose DNA and amino acid sequences encoding OP-1 (BMP-7), and methods for OP-1 recombinant expression. For an alignment of BMP-2, BMP-4, BMP-5, BMP-6 and OP-1 (BMP-7) amino acid sequences, see WO 95/16034.

DNA sequences encoding BMP-8 are disclosed in WO 91/18098, and DNA sequences encoding BMP-9 in WO 93/00432. DNA and deduced amino acid sequences encoding BMP-10 and BMP-11 are disclosed in WO 94/26893, and WO 94/26892, respectively. DNA and deduced amino acid sequences for BMP-12 (CDMP-3) and BMP-13 (CDMP-2) are disclosed in WO 95/16035. DNA and deduced amino acid sequence for BMP-14 (CDMP-1) is discussed in WO 96/14335.

The above patent disclosures, which describe DNA and amino acid sequences, and methods for producing the BMPs and OPs encoded by those sequences, are incorporated herein by reference.

To clone genes which encode new BMPS, OPs and other morphogenic proteins identified in extracts by bioassay, methods entailing “reverse genetics” may be employed. Such methods start with a protein of known or unknown function to obtain the gene which encodes that protein. Standard protein purification techniques may be used as an initial step in cloning the gene by reverse genetics. If enough protein can be purified to obtain a partial amino acid sequence, a degenerate DNA probe capable of hybridizing to the DNA sequence that encodes that partial amino acid sequence may be designed, synthesized and used as a probe to isolate full-length clones that encode that or a related morphogenic protein.

Alternatively, a partially-purified extract containing the morphogenic agent may be used to raise antibodies directed against that agent using immunological procedures well known in the art. Morphogenic protein-specific antibodies may then be used as a probe to screen expression libraries made from cDNAs (see, e.g., Broome and Gilbert, Proc. Natl. Acad. Sci. U.S.A., 75, pp. 2746-49 (1978; Young and Davis, Proc. Natl. Acad. Sci. U.S.A., 80, pp. 31-35 (1983)).

For cloning and expressing new BMPs, OPs and other morphogenic proteins identified based on DNA sequence homology, the homologous sequences may be cloned and sequenced using standard recombinant DNA techniques. With the DNA sequence available, a DNA fragment encoding the morphogenic protein may be inserted into an expression vector selected to work in conjunction with a desired host expression system. The DNA fragment is cloned into the vector such that its transcription is controlled by a heterologous promoter in the vector, preferably a promoter which may be optionally regulated.

Some host-vector systems that are appropriate for the recombinant expression of BMPs and OPs are disclosed in the references cited above. Useful host cells include but are not limited to bacteria such as E. coli, yeasts such as Saccharomyces and Picia, insect-baculovirus cell system, and primary, transformed or immortalized eukaryotic cells in culture. Preferred eukaryotic host cells include CHO, COS and BSC cells (see below).

An appropriate vector is selected according to the host system selected. Useful vectors include but are not limited to plasmids, cosmids, bacteriophage, insect and animal viral vectors, including retroviruses, and other single and double-stranded DNA viruses.

In one embodiment, the morphogenic proteins used in the methods of this invention may be derived from a recombinant DNA molecule expressed in a prokaryotic host (Exanple 2A). Using recombinant DNA techniques, various fusion genes have been constructed to induce recombinant expression of naturally-sourced osteogenic sequences in E. coli (see, e.g., Oppermann et al., U. S. Pat. No. 5,354,557, incorporated herein by reference). Using analogous procedures, DNAs comprising truncated forms of naturally-sourced morphogenic sequences may be prepared as fusion constructs linked by the acid labile cleavage site (Asp-Pro) to a leader sequence (such as the “MLE leader”) suitable for promoting expression in E. coli.

In another embodiment, the morphogenic proteins used in this invention are expressed using a mammalian host/vector system (Example 2B). It may be preferable to recombinantly produce a mammalian protein for therapeutic uses in mammalian cell culture systems in order to produce a protein whose structure resembles more closely that of the natural material. Recombinant protein production in mammalian cells requires the establishment of appropriate cells and cell lines that are easy to transfect, are capable of stably maintaining foreign DNA with an unrearranged sequence, and which have the necessary cellular components for efficient transcription, translation, post-translational modification and secretion of the protein. In addition, a suitable vector carrying the gene of interest is necessary.

DNA vector design for transfection into mammalian cells should include appropriate sequences to promote expression of the gene of interest, including: appropriate transcription initiation, termination and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion.

Preferred DNA vectors also include a marker gene and means for amplifying the copy number of the gene of interest. DNA vectors may also comprise stabilizing sequences (e.g., ori- or ARS-like sequences and telomere-like sequences), or may alternatively be designed to favor directed or non-directed integration into the host cell genome.

Substantial progress in the development of mammalian cell expression systems has been made in the last decade and many aspects of the system are well characterized. A detailed review of the production of foreign proteins in mammalian cells, including useful cells, protein expression-promoting sequences, marker genes, and gene amplification methods, is disclosed in M. M. Bendig, Genetic Engineering, 7, pp. 91-127 (1988).

Particular details of the transfection, expression and purification of recombinant proteins are well documented and are understood by those of skill in the art. Further details on the various technical aspects of each of the steps used in recombinant production of foreign genes in mammalian cell expression systems can be found in a number of texts and laboratory manuals in the art. See, e.g., F. M. Ausubel et al., ed., Current Protocols in Molecular Biology, John Wiley & Sons, New York (1989).

Briefly, among the best characterized transcription promoters useful for expressing a foreign gene in a particular mammalian cell are the SV40 early promoter, the adenovirus major late promoter (AdMLP), the mouse metallothionein-I promoter (mMT-I), the Rous sarcoma virus (RSV) long terminal repeat (LTR), the mouse mammary tumor virus long terminal repeat (MMTV-LTR), and the human cytomegalovirus major intermediate-early promoter (hCMV). The DNA sequences for all of these promoters are known in the art and are available commercially.

One method of gene amplification in mammalian cell systems is the use of the selectable dihydrofolate reductase (DHFR) gene in a dhfr-cell line. Generally, the DHFR gene is provided on the vector carrying the gene of interest, and addition of increasing concentrations of the cytotoxic drug methotrexate (MTX) leads to amplification of the DHFR gene copy number, as well as that of the physically-associated gene of interest. DHFR as a selectable, amplifiable marker gene in transfected chinese hamster ovary cell lines (CHO cells) is particularly well characterized in the art. Other useful amplifiable marker genes include the adenosine deaminase (ADA) and glutamine synthetase (GS) genes.

In a preferred expression system, gene amplification is further enhanced by modifying marker gene expression regulatory sequences (e.g., enhancer, promoter, and transcription or translation initiation sequences) to reduce the levels of marker protein produced. Lowering the level of DHFR transcription increases the DHFR gene copy number (and the physically-associated gene) to enable the transfected cell to adapt to growth in even low levels of methotrexate (e.g., 0.1 μM MTX). Preferred expression vectors such as pH754 and pH752 (Oppermann et al., U.S. Pat. No. 5,354,557, FIGS. 19C and D), have been manipulated using standard recombinant DNA technology, to create a weak DHFR promoter. As will be appreciated by those skilled in the art, other useful weak promoters, different from those disclosed and preferred herein, can be constructed using standard vector construction methodologies. In addition, other, different regulatory sequences also can be modified to achieve the same effect.

Another gene amplification scheme relies on the temperature sensitivity (ts) of BSC40-tsA58 cells transfected with an SV40 vector. Temperature reduction to 33° C. stabilizes the temperature sensitive SV40 T antigen, which leads to the excision and amplification of the integrated transfected vector DNA thereby amplifying the physically associated gene of interest.

The choice of cells/cell lines is also important and depends on the needs of the skilled practitioner. Monkey kidney cells (COS) provide high levels of transient gene expression providing a useful means for rapidly testing vector construction and the expression of cloned genes. COS cells are transfected with a simian virus 40 (SV40) vector carrying the gene of interest. The transfected COS cells eventually die, thus preventing the long term production of the desired protein product. However, transient expression does not require the time consuming process required for the development of stable cell lines.

CHO cells are capable of successfully expressing a wide variety of proteins from a broad range of cell types. Thus, while the glycosylation pattern on a recombinant protein produced in a mammalian cell expression system may not be identical to the natural protein, the differences in oligosaccharide side chains are often not essential for biological activity of the expressed protein.

Several different mammalian cell expression systems may be used to express recombinant morphogenic proteins to use according to this invention. Stable cell lines have been developed using CHO cells and a temperature-sensitive (ts) strain of BSC cells (simian kidney cells, BSC40-tsA58; Biotechnology, 6, pp. 1192-96 (1988)) for the long term production of osteogenic protein OP-1. Among established cell lines, CHO cells may be the best characterized to date, and are a preferred cell line for mammalian cell expression of recombinant morphogenic proteins (Example 2b).

Two different promoters were found most useful to transcribe human osteogenic protein sequences (hOP1; SEQ. ID NO. 1): the CMV promoter and the MMTV promoter, boosted by the enhancer sequence from the Rous sarcoma virus LTR. The mMT promoter (mouse metallothionein promoter) and the SV40 late promoter have also been tested. Several selection marker genes such as neo (neomycin) and DHFR are used.

Restriction maps and sources of various exemplary expression vectors designed for OP-1 expression in mammalian cells have been described in Oppermann et al., U.S. Pat. No. 5,354,557, incorporated herein by reference (see Example 2B). Each of these vector constructs employs a full-length human OP-1 CDNA sequence cloned into a conventional pUC vector (pUC-18).

It will be appreciated by those of skill in the art that DNA sequences encoding truncated forms of osteogenic protein may also be used, provided that the expression vector or host cell then provides the sequences necessary to direct processing and secretion of the expressed protein.

Recombinant OP-1 has been expressed in three different cell expression systems: COS cells for rapidly screening the functionality of the various expression vector constructs, CHO cells for the establishment of stable cell lines, and BSC40-tsA58 cells as an alternative means of producing recombinant OP-1 protein. The CHO cell expression system disclosed herein is contemplated to be the best mode currently known for long-term recombinant OP-1 production in mammalian cells (see Example 2B). The morphogenic proteins of this invention may also be expressed in progenitor cells. In some embodiments, the progenitor cells include but are not limited to bone progenitor cells, cartilage progenitor cells, tendon progenitor cells or ligament progenitor cells. For example, the nucleic acids encoding the morphogenic proteins or vectors comprising them may be transfected into the progenitor cells. These cells are then cultured under appropriate conditions for growth. The cultured progenitor cells are then administered to the target locus. Alternatively, the nucleic acid encoding the morphogenic protein or the vector comprising it may be administered directly to the target locus.

As discussed above, several bone-derived osteogenic proteins (OPs) and BMPs are found as homo- and heterodimers comprising interchain disulfide bonds in their active forms. Methods for co-expressing and assembling heteromeric polypeptide subunits in a host have been described (see, e.g., WO 93/09229, which is incorporated herein by reference). BMP-2, BMP-4, BMP-6 and BMP-7 (OP-1)—originally isolated from bone—are bioactive as either homodimers or heterodimers.

In addition, methods for making amino acid substitution mutations in BMPs and OPs that favor refolding and/or assembling subunits into forms that exhibit greater morphogenic activity have also been described (U.S. Pat. No. 5,399,677, which is incorporated herein by reference).

As the skilled practitioner will appreciate, the preferred combination of morphogenic proteins of this invention will depend in part on the tissue type to be generated and on the selected implantation or treatment site. These variables may be tested empirically.

Testing Putative Combinations of Morphogenic Proteins

To identify combinations of morphogenic proteins that may be useful in the methods of the present invention, an appropriate assay must be selected. Initially, it is preferable to perform in vitro assays to identify a combination of morphogenic proteins that is useful in the methods of the present invention. A useful in vitro assay is one which monitors a nucleic acid or protein marker whose expression is known to correlate with the associated cell differentiation pathway.

Examples 3 and 4 describe experiments using the osteogenic protein OP-1 to identify and to optimize an effective concentration of a second morphogenic protein. As described above, OP-1 is known to have osteogenic activity. Thus an in vitro assay looking at the expression of either an osteo-associated marker in appropriately corresponding progenitor cells can be used to identify a second morphogenic protein that function in concert with OP-1.

Testing Putative Combinations of Morphogenic Proteins Using Morphogenic Assays

A preferred assay for testing the potential of a second morphogenic protein with OP-1 for inducing osteogenic activity is the alkaline phosphatase (AP) enzymatic assay. AP is an osteoblast differentiation marker in primary C2C12 cells, a pluripotent mesenchymal precursor cell line suitable for studying the early stages of osteoblast differentiation during bone formation in muscle cells. The OP-1-stimulated AP activity is the result of increased steady-state AP mRNA levels as measured by Northern analysis. The procedure is generally as follows.

First, the second morphogenic protein is identified by picking one or more concentrations of a second morphogenic protein and testing them alone or in the presence of a first morphogenic protein (Examples 3 and 4). Second, the amount of the second morphogenic protein required to achieve optimal, preferably synergistic, tissue induction in concert with the first morphogenic protein is determined by generating a dose response curve (Example 3).

Levels for additional biochemical markers for bone cell differentiation may be measured to assay for synergistic effects of OP-1 with a second morphogenic protein. Other bone cell differentiation markers include but are not limited to: type I collagen, osteocalcin, osteopontin, bone sialoprotein and PTH-dependent cAMP levels.

Once a first morphogenic protein/second morphogenic protein pair has been identified, it is desirable to identify the relative amounts of each component that are required to effectuate optimal levels of tissue inductive activity when the two components work in concert. This is done by assaying the tissue inductive activity produced when the concentration of each component is systematically varied independently from the other. The result of such a study is a dose response curve for a given first morphogenic protein/second morphogenic protein pair.

It may not hold true for every first morphogenic protein/second morphogenic protein combination that co-administration is optimal for inducing morphogenic activity. The procedures described herein can be used by the skilled practitioner to optimize an administration protocol for a given first morphogenic protein/second morphogenic protein combination to induce a selected tissue type at a selected treatment site.

The procedure described herein may be used generally with any selected first morphogenic protein to test putative second morphogenic protein (Example 4). Initially, the first morphogenic protein or agent is used to identify and then to optimize conditions for an assay that accurately represents the induction of a particular type of cell differentiation pathway associated with tissue formation. As described above, an in vitro assay that is representative of the induction of the desired tissue type is preferred at this stage. The assay may monitor mRNA or protein levels as a function of time or at a set time after administration of the morphogenic protein to cells or a tissue explant.

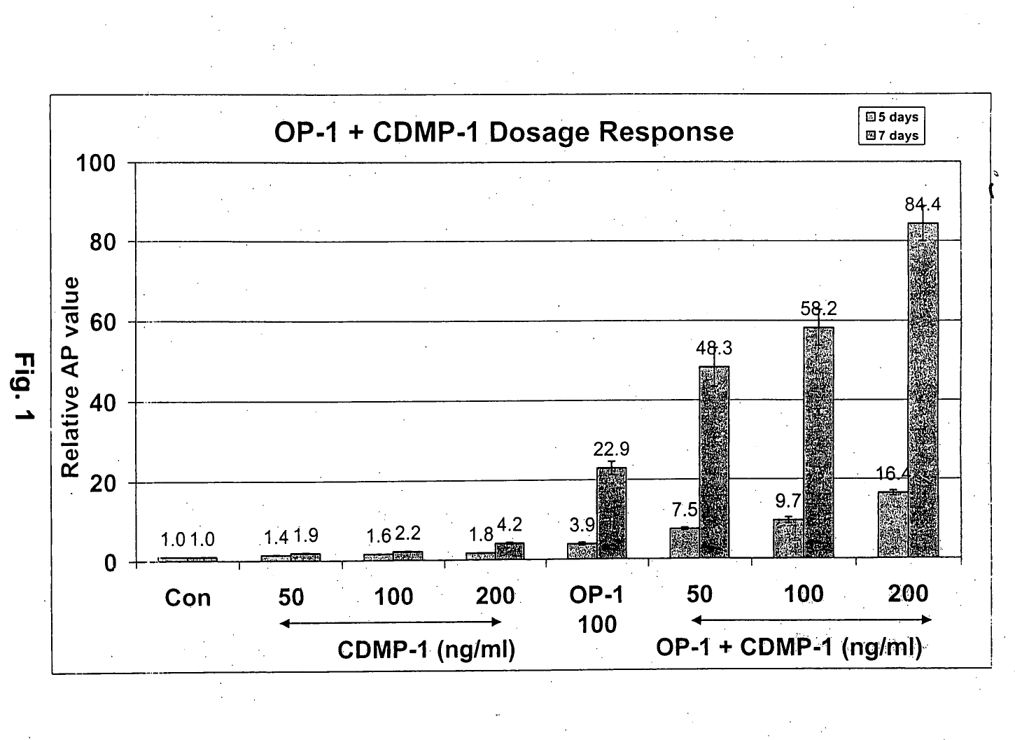

In general, at least about 1 ng/ml of the first morphogenic protein is combined with at least about 1 ng/ml of a second morphogenic protein to observe an increase in the morphogenic activity. Preferred concentration ranges for combinations of osteogenic protein OP-1 and a second morphogenic protein such as a CDMP, in inducing bone and cartilage formation, as determined in experiments such as those shown in FIGS. 1-3.

It will be appreciated by those skilled in the art that the preferred concentration range of second morphogenic protein in a particular assay may vary depending on the concentration of the first morphogenic protein selected. Systematic variation of the relative concentrations of the first morphogenic protein and the second morphogenic protein should thus be performed to optimize concentration ratios of the two factors.

It is envisioned further that other second morphogenic proteins that are capable of stimulating the tissue inductive activity of the first morphogenic protein which may be identified according to the methods herein may also be optimized for activity by producing variant forms of that second morphogenic protein which have altered abilities to interact with other cellular proteins such as target and/or competitive receptors, inhibitory and/or stimulatory binding proteins and the like, altered stabilities, or altered localization characteristics. Methods to produce variant forms of proteins by chemical modifications, mutagenesis and recombinant DNA technology are known to those of skill in the art. The variant forms of the second morphogenic protein may then be tested and compared with the original second morphogenic protein for the ability to stimulate cell proliferation and/or differentiation in the presence of the first morphogenic protein according to the methods set forth herein. In this way, first morphogenic protein/second morphogenic protein combinations may be optimized to function in a desired way in the particular therapeutic context for which they are ultimately intended.

Based on first morphogenic protein/second morphogenic protein dose response curves in morphogenic and/or mitogenic assays such as those discussed above, compositions comprising a first morphogenic protein and a second morphogenic protein may be formulated at various concentration ratios and tested in a bioassay selected to represent the tissue inductive activity which will ultimately be used in the tissue treatment. The preferred assay is ultimately an ex vivo or in vivo tissue induction bioassay such as those described in Examples 3-6.

Pharmaceutical Compositions

The pharmaceutical compositions provided by this invention comprise at least two morphogenic proteins in combination for inducing tissue formation when administered or implanted into a patient. The compositions of this invention will be administered at an effective dose to induce the particular type of tissue at the treatment site selected according to the particular clinical condition addressed. Determination of a preferred pharmaceutical formulation and a therapeutically efficient dose regiment for a given application is well within the skill of the art taking into consideration, for example, the administration mode, the condition and weight of the patient, the extent of desired treatment and the tolerance of the patient for the treatment.

Doses expected to be suitable starting points for optimizing treatment regiments are based on the results of in vitro assays (e.g., Examples 3-5). Based on the results of such assays, a range of suitable morphogenic protein concentration ratios can be selected to test at a treatment site in animals and then in humans.

Administration of the morphogenic proteins of this invention, including isolated and purified forms of morphogenic protein complexes, their salts or pharmaceutically acceptable derivatives thereof, may be accomplished using any of the conventionally accepted modes of administration of agents which exhibit immunosuppressive activity.

The pharmaceutical compositions comprising the morphogenic proteins of this invention may be in a variety of forms. These include, for example, solid, semi-solid and liquid dosage forms such as tablets, pills, powders, liquid solutions or suspensions, suppositories, and injectable and infusible solutions. The preferred form depends on the intended mode of administration and therapeutic application and may be selected by one skilled in the art. Modes of administration may include oral, parenteral, subcutaneous, intravenous, intralesional or topical administration. In most cases, the pharmaceutical compositions of this invention will be administered in the vicinity of the treatment site in need of tissue regeneration or repair.

The pharmaceutical compositions comprising the morphogenic proteins of this invention may, for example, be placed into sterile, isotonic formulations with or without cofactors which stimulate uptake or stability. The formulation is preferably liquid, or may be lyophilized powder. For example, the morphogenic proteins of this invention may be diluted with a formulation buffer comprising 5.0 mg/ml citric acid monohydrate, 2.7 mg/ml trisodium citrate, 41 mg/ml mannitol, 1 mg/ml glycine and 1 mg/ml polysorbate 20. This solution can be lyophilized, stored under refrigeration and reconstituted prior to administration with sterile Water-For-Injection (USP).

The compositions also will preferably include conventional pharmaceutically acceptable carriers well known in the art (see for example Remington's Pharmaceutical Sciences, 16th Edition, 1980, Mac Publishing Company). Such pharmaceutically acceptable carriers may include other medicinal agents, carriers, genetic carriers, adjuvants, excipients, etc., such as human serum albumin or plasma preparations. The compositions are preferably in the form of a unit dose and will usually be administered as a dose regiment that depends on the particular tissue treatment.

The pharmaceutical compositions of this invention may also be administered in conjunction with a morphogenic device using, for example, microspheres, liposomes, other microparticulate delivery systems or sustained release formulations placed in, near, or otherwise in communication with affected tissues or the bloodstream bathing those tissues (see morphogenic devices, below).

Liposomes containing the morphogenic proteins of this invention can be prepared by well-known methods (See, e.g. DE 3,218,121; Epstein et al., Proc. Natl. Acad. Sci. U.S.A., 82, pp. 3688-92 (1985); Hwang et al., Proc. Natl. Acad. Sci. U.S.A., 77, pp. 4030-34 (1980); U.S. Pat. Nos. 4,485,045 and 4,544,545). Ordinarily the liposomes are of the small (about 200-800 Angstroms) unilamellar type in which the lipid content is greater than about 30 mol. % cholesterol. The proportion of cholesterol is selected to control the optimal rate of morphogenic protein release.

The morphogenic protein combinations of this invention may also be attached to liposomes containing other biologically active molecules such as immunosuppressive agents, cytokines, etc., to modulate the rate and characteristics of tissue induction. Attachment of morphogenic proteins to liposomes may be accomplished by any known cross-linking agent such as heterobifunctional cross-linking agents that have been widely used to couple toxins or chemotherapeutic agents to antibodies for targeted delivery. Conjugation to liposomes can also be accomplished using the carbohydrate-directed cross-linking reagent 4-(4-maleimidophenyl) butyric acid hydrazide (MPBH) (Duzgunes et al., J. Cell. Biochem. Abst. Suppl. 16E 77 (1992)).

Morphogenic Devices

The morphogenic devices of this invention comprise a first morphogenic protein and at least a second morphogenic protein dispersed in an implantable biocompatible carrier material that functions as a suitable delivery or support system for the compounds. The morphogenic devices optionally comprise additional morphogenic proteins. Suitable examples of sustained release carriers include semipermeable polymer matrices in the form of shaped articles such as suppositories or capsules. Implantable or microcapsular sustained release matrices include polylactides (U.S. Pat. No. 3,773,319; EP 58,481), copolymers of L-glutamic acid and ethyl-L-glutamate (Sidman et al., Biopolymers, 22, pp. 547-56 (1985)); poly(2-hydroxyethyl-methacrylate) or ethylene vinyl acetate (Langer et al., J. Biomed. Mater. Res., 15, pp. 167-277 (1981); Langer, Chem. Tech., 12, pp. 98-105 (1982)).

In one embodiment of this invention, the carrier of the morphogenic device comprises a biocompatible matrix made up of particles or porous materials. The pores are preferably of a dimension to permit progenitor cell migration and subsequent differentiation and proliferation. Various matrices known in the art can be employed (see, e.g., U.S. Pat. Nos. 4,975,526; 5,162,114; 5,171,574 and WO 91/18558, which are herein incorporated by reference).

The particle size should be within the range of 70 μm-850 μm, preferably 70 μm-420 μm, most preferably 150 μm-420 μm. The matrix may be fabricated by close packing particulate material into a shape spanning the particular tissue defect to be treated. Alternatively, a material that is biocompatible, and preferably biodegradable in vivo may be structured to serve as a temporary scaffold and substratum for recruitment of migratory progenitor cells, and as a base for their subsequent anchoring and proliferation.

Useful matrix materials comprise, for example, collagen; homopolymers or copolymers of glycolic acid, lactic acid, and butyric acid, including derivatives thereof; and ceramics, such as hydroxyapatite, tricalcium phosphate and other calcium phosphates. Various combinations of these or other suitable matrix materials also may be useful as determined by the assays set forth herein.

Currently preferred carriers include particulate, demineralized, guanidine-extracted, species-specific (allogenic) bone, and specially treated particulate, protein-extracted, demineralized xenogenic bone (see Example 6). Optionally, such xenogenic bone powder matrices also may be treated with proteases such as trypsin. Preferably, the xenogenic matrices are treated with one or more fibril modifying agents to increase the intraparticle intrusion volume (porosity) and surface area. Useful modifying agents include solvents such as dichloromethane, trichloroacetic acid, acetonitrile and acids such as trifluoroacetic acid and hydrogen fluoride. The currently preferred fibril-modifying agent useful in formulating the matrices of this invention is a heated aqueous medium, preferably an acidic aqueous medium having a pH less than about pH 4.5, most preferably having a pH within the range of about pH 2-pH 4. A currently preferred heated acidic aqueous medium is 0.1% acetic acid which has a pH of about 3. Heating demineralized, delipidated, guanidine-extracted bone collagen in an aqueous medium at elevated temperatures (e.g., in the range of about 37° C.-65° C., preferably in the range of about 45° C.-60° C.) for approximately one hour generally is sufficient to achieve the desired surface morphology. Although the mechanism is not clear, it is hypothesized that the heat treatment alters the collagen fibrils, resulting in an increase in the particle surface area.

Demineralized guanidine-extracted xenogenic bovine bone comprises a mixture of additional materials that may be fractionated further using standard biomolecular purification techniques. For example, chromatographic separation of extract components followed by addition back to active matrix of the various extract fractions corresponding to the chromatogram peaks may be used to improve matrix properties by fractionating away inhibitors of bone or tissue-inductive activity.

The matrix may also be substantially depleted in residual heavy metals. Treated as disclosed herein, individual heavy metal concentrations in the matrix can be reduced to less than about 1 ppm.

One skilled in the art may create a biocompatible matrix of choice having a desired porosity or surface microtexture useful in the production of morphogenic devices to promote bone or other tissue induction, or as a biodegradable sustained release implant. In addition, synthetically formulated matrices, prepared as disclosed herein, may be used.

General Consideration of Matrix Properties

The currently preferred carrier material is a xenogenic bone-derived particulate matrix treated as described herein. This carrier may be replaced by either a biodegradable-synthetic or a synthetic-inorganic matrix (e.g., hydroxyapatite (HAP), collagen, carboxymethyl-cellulose, tricalcium phosphate or polylactic acid, polyglycolic acid, polybutyric acid and various copolymers thereof).

Matrix geometry, particle size, the presence of surface charge, and the degree of both intra- and inter-particle porosity are all important to successful matrix performance. Studies have shown that surface charge, particle size, the presence of mineral, and the methodology for combining matrix and morphogenic proteins all play a role in achieving successful tissue induction.

For example, in bone formation using osteogenic protein OP-1 and a second morphogenic protein, perturbation of the matrix charge by chemical modification can abolish bone inductive responses. Particle size influences the quantitative response of new bone; particles between 70 μm and 420 μm elicit the maximum response. Contamination of the matrix with bone mineral will inhibit bone formation. Most importantly, the procedures used to formulate osteogenic protein and a second morphogenic protein onto the matrix are extremely sensitive to the physical and chemical state of both the proteins and the matrix.

The sequential cellular reactions in the interface of the bone matrix/osteogenic protein implants are complex. The multistep cascade includes: binding of fibrin and fibronectin to implanted matrix, migration and proliferation of mesenchymal cells, differentiation of the progenitor cells into chondroblasts, cartilage formation, cartilage calcification, vascular invasion, bone formation, remodeling, and bone marrow differentiation.

A successful carrier for the morphogenic protein combination should perform several important functions. It should act as a slow release delivery system of the morphogenic proteins, protect the morphogenic proteins from non-specific proteolysis, and should accommodate each step of the cellular responses involved in progenitor cell induction during tissue development.

In addition, selected materials must be biocompatible in vivo and preferably biodegradable; the carrier preferably acts as a temporary scaffold until replaced completely by new bone or tissue. Polylactic acid (PLA), polyglycolic acid (PGA), and various combinations have different dissolution rates in vivo. In bones, the dissolution rates can vary according to whether the implant is placed in cortical or trabecular bone.

The preferred osteogenic device matrix material, prepared from xenogenic bone and treated as disclosed herein, produces an implantable material useful in a variety of clinical settings. In addition to its use as a matrix for bone formation in various orthopedic, periodontal, and reconstructive procedures, the matrix also may be used as a sustained release carrier, or as a collagenous coating for orthopedic or general prosthetic implants.

The matrix may be shaped as desired in anticipation of surgery or shaped by the physician or technician during surgery. It is preferred to shape the matrix to span a tissue defect and to take the desired form of the new tissue. In the case of bone repair of a non-union defect, for example, it is desirable to use dimensions that span the non-union. Rat studies show that the new bone is formed essentially having the dimensions of the device implanted. Thus, the material may be used for topical, subcutaneous, intraperitoneal, or intramuscular implants. In bone formation procedures, the material is slowly absorbed by the body and is replaced by bone in the shape of or very nearly the shape of the implant.

The matrix may comprise a shape-retaining solid made of loosely-adhered particulate material, e.g., collagen. It may also comprise a molded, porous solid, or simply an aggregation of close-packed particles held in place by surrounding tissue. Masticated muscle or other tissue may also be used. Large allogenic bone implants can act as a carrier for the matrix if their marrow cavities are cleaned and packed with particles comprising dispersed morphogenic protein combinations. The matrix may also take the form of a paste or a hydrogel.

When the carrier material comprises a hydrogel matrix, it refers to a three dimensional network of cross-linked hydrophilic polymers in the form of a gel substantially composed of water, preferably but not limited to gels being greater than 90% water. Hydrogel matrices can carry a net positive or net negative charge, or may be neutral. A typical net negative charged matrix is alginate. Hydrogels carrying a net positive charge may be typified by extracellular matrix components such as collagen and laminin. Examples of commercially available extracellular matrix components include Matrigel™ and Vitrogen™. An example of a net neutral hydrogel is highly crosslinked polyethylene oxide, or polyvinyalcohol.

Various growth factors, cytokines, hormones, trophic agents and therapeutic compositions including antibiotics and chemotherapeutic agents, enzymes, enzyme inhibitors and other bioactive agents also may be adsorbed onto or dispersed within the carrier material comprising combinations of the morphogenic proteins, and will also be released over time at the implantation site as the matrix material is slowly absorbed.

Other Tissue-Specific Matrices

In addition to the naturally-derived bone matrices described above, useful matrices may also be formulated synthetically by adding together reagents that have been appropriately modified. One example of such a matrix is the porous, biocompatible, in vivo biodegradable synthetic matrix disclosed in WO 91/18558, the disclosure of which is hereby incorporated by reference.

Briefly, the matrix comprises a porous crosslinked structural polymer of biocompatible, biodegradable collagen, most preferably tissue-specific collagen, and appropriate, tissue-specific glycosaminoglycans as tissue-specific cell attachment factors. Bone tissue-specific collagen (e.g., Type I collagen) derived from a number of sources may be suitable for use in these synthetic matrices, including soluble collagen, acid-soluble collagen, collagen soluble in neutral or basic aqueous solutions, as well as those collagens which are commercially available. In addition, Type II collagen, as found in cartilage, also may be used in combination with Type I collagen.

Glycosaminoglycans (GAGs) or mucopolysaccharides are polysaccharides made up of residues of hexoamines glycosidically bound and alternating in a more-or-less regular manner with either hexouronic acid or hexose moieties. GAGs are of animal origin and have a tissue specific distribution (see, e.g., Dodgson et al., in Carbohydrate Metabolism and its Disorders, Dickens et al., eds., Vol. 1, Academic Press (1968)). Reaction with the GAGs also provides collagen with another valuable property, i.e., inability to provoke an immune reaction (foreign body reaction) from an animal host.

Useful GAGs include those containing sulfate groups, such as hyaluronic acid, heparin, heparin sulfate, chondroitin 6-sulfate, chondroitin 4-sulfate, dermatan sulfate, and keratin sulfate. For osteogenic devices, chondroitin 6-sulfate currently is preferred. Other GAGs also may be suitable for forming the matrix described herein, and those skilled in the art will either know or be able to ascertain other suitable GAGs using no more than routine experimentation. For a more detailed description of mucopolysaccharides, see Aspinall, Polysaccharides, Pergamon Press, Oxford (1970).

Collagen can be reacted with a GAG in aqueous acidic solutions, preferably in diluted acetic acid solutions. By adding the GAG dropwise into the aqueous collagen dispersion, coprecipitates of tangled collagen fibrils coated with GAG results. This tangled mass of fibers then can be homogenized to form a homogeneous dispersion of fine fibers and then filtered and dried.

Insolubility of the collagen-GAG products can be raised to the desired degree by covalently cross-linking these materials, which also serves to raise the resistance to resorption of these materials. In general, any covalent G60 cross-linking method suitable for cross-linking collagen also is suitable for cross-linking these composite materials, although cross-linking by a dehydrothermal process is preferred.

When dry, the cross-linked particles are essentially spherical with diameters of about 500 μm. Scanning electron microscopy shows pores of about 20 μm on the surface and 40 μm on the interior. The interior is made up of both fibrous and sheet-like structures, providing surfaces for cell attachment. The voids interconnect, providing access to the cells throughout the interior of the particle. The material appears to be roughly 99.5% void volume, making the material very efficient in terms of the potential cell mass that can be grown per gram of microcarrier.

Another useful synthetic matrix is one formulated from biocompatible, in vivo biodegradable synthetic polymers, such as those composed of glycolic acid, lactic acid and/or butyric acid, including copolymers and derivatives thereof. These polymers are well described in the art and are available commercially. For example, polymers composed of polylactic acid (e.g., MW 100 ka), 80% polylactide/20% glycoside or poly 3-hydroxybutyric acid (e.g., MW 30 ka) all may be purchased from PolySciences, Inc. The polymer compositions generally are obtained in particulate form and the morphogenic devices preferably fabricated under nonaqueous conditions (e.g., in an ethanol-trifluoroacetic acid solution, EtOH/TFA) to avoid hydrolysis of the polymers. In addition, one can alter the morphology of the particulate polymer compositions, for example to increase porosity, using any of a number of particular solvent treatments known in the art.

Preparation of Morphogenic Device

The naturally-sourced, synthetic and recombinant morphogenic proteins as set forth above, as well as other constructs, can be combined and dispersed in a suitable matrix preparation using any of the methods described. In general, about 1-1000 ng of each active morphogenic protein is combined with 25 mg of the inactive carrier matrix for rat bioassays. In larger animals, typically about 0.8-1 mg of each active morphogenic protein per gram of carrier is used. The optimal ratios of a first morphogenic protein to a second morphogenic protein for a specific combination and tissue type may be determined empirically by those of skill in the art according to the procedures set forth herein. Greater amounts may be used for large implants.

Prosthetic Devices

In another embodiment of this invention, an implantable prosthetic device comprising a combination of at least two morphogenic proteins is provided. Any prosthetic implant selected for a particular treatment by the skilled practitioner may be used in combination with a composition comprising at least two morphogenic proteins according to this invention. The prosthesis may be made from a material comprising metal or ceramic. Preferred prosthetic devices are selected from the group consisting of a hip device, a screw, a rod and a titanium cage for spine fusion.

The morphogenic protein composition is disposed on the prosthetic implant on a surface region that is implantable adjacent to a target tissue in the mammal. Preferably, the mammal is a human patient. The composition is disposed on the surface of the implant in an amount sufficient to promote enhanced tissue growth into the surface. The amount of the composition sufficient to promote enhanced tissue growth may be determined empirically by those of skill in the art using bioassays such as those described herein and in Rueger et al., U.S. Pat. No. 5,344,654, which is incorporated herein by reference. Preferably, animal studies are performed to optimize the concentration of the composition components before a similar prosthetic device is used in the human patient. Such prosthetic devices will be useful for repairing orthopedic defects, injuries or anomalies in the treated mammal.

Thus this invention also provides a method for promoting in vivo integration of an implantable prosthetic device into a target tissue of a mammal comprising the steps of providing on a surface of the prosthetic device a composition comprising at least one osteogenic protein and at least two morphogenic proteins, and implanting the device in a mammal at a locus where the target tissue and the surface of the prosthetic device are maintained at least partially in contact for a time sufficient to permit enhanced tissue growth between the target tissue and the device.

Utility of Morphogenic Compositions and Devices

The morphogenic compositions and devices comprising at least two morphogenic proteins disclosed herein will permit the physician to treat a variety of tissue injuries, tissue degenerative or disease conditions and disorders that can be ameliorated or remedied by localized, stimulated tissue regeneration or repair.

The morphogenic compositions and devices of this invention may be used to induce local tissue formation from a progenitor cell in a mammal by implanting the device at a locus accessible to at least one progenitor cell of the mammal. The morphogenic compositions and devices used in the methods of this invention include the use of the morphogenic proteins themselves, the nucleic acids encoding them, vectors comprising the nucleic acids encoding them, and cells comprising the vectors and nucleic acids encoding the morphogenic proteins. The morphogenic devices of this invention may be used alone or in combination with other therapies for tissue repair and regeneration.

The tissue specificity of the particular morphogenic protein—or combination of morphogenic proteins with other biological factors—will determine the cell types or tissues that will be amenable to such treatments and can be selected by one skilled in the art. The ability to enhance a first morphogenic protein-induced tissue regeneration by co-administering a second morphogenic protein according to the present invention is thus not believed to be limited to any particular cell-type or tissue. It is envisioned that the invention as disclosed herein can be practiced to enhance the activities of new morphogenic proteins and to enhance new tissue inductive functions as they are discovered in the future.

The osteogenic compositions and devices comprising at least two morphogenic proteins will permit the physician to obtain predictable bone, cartilage, tendon and/or ligament formation using less osteogenic protein to achieve at least about the same extent of bone, cartilage, tendon or ligament formation. The osteogenic compositions and devices of this invention may be used to treat more efficiently and/or effectively all of the injuries, anomalies and disorders that have been described in the prior art of osteogenic devices. These include, for example, forming local bone in fractures, non-union fractures, fusions and bony voids such as those created in tumor resections or those resulting from cysts; treating acquired and congenital craniofacial and other skeletal or dental anomalies (see e.g., Glowacki et al., Lancet, 1, pp. 959-63 (1981)); performing dental and periodontal reconstructions where lost bone replacement or bone augmentation is required such as in a jaw bone; and supplementing alveolar bone loss resulting from periodontal disease to delay or prevent tooth loss (see e.g., Sigurdsson et al., J. Periodontol., 66, pp. 511-21 (1995)).

An osteogenic device of this invention which comprises a matrix comprising allogenic bone may also be implanted at a site in need of bone replacement to accelerate allograft repair and incorporation in a mammal.

The morphogenic compositions and devices of this invention will be useful in treating certain congenital diseases and developmental abnormalities of cartilage, bone and other tissues. For example, homozygous OP-1 (BMP-7)-deficient mice die within 24 hours after birth due to kidney failure (Luo et al., J. Bone Min. Res., 10 (Supp. 1), pp. S163 (1995)). Kidney failure in these mice is associated with the failure to form renal glomeruli due to lack of mesenchymal tissue condensation. OP-1-deficient mice also have various skeletal abnormalities associated with their hindlimbs, rib cage and skull, are polydactyl, and exhibit aberrant retinal development. These results, in combination with those discussed above concerning the ability of OP-1 to induce differentiation into dorsal neural cell fates, indicate that OP-1 plays an important role in epithelial-mesenchymal interactions during development. Similarly, mice carrying a mutated GDF-5 (CDMP-1) gene shows a limb brachypodism phenotype and disruption of tail formation (Polinkovsky et al., Nat Genet, 17, pp. 18-19 (1997); Clark et al., Connect Tissue Res, 42, pp. 175-186 (2001)). Furthermore, GDF-5 deficiency in mice alters the ultrastructure, mechanical properties, and composition of the Achilles tendon and the cortical bone (Mikic et al., J Orthop Res, 19, pp. 365-371 (2001); Mikic et al., Bone, 30, pp. 733-737 (2002)). Similarly, patients with a mutated human CDMP-1 gene shows the Hunter-Thompson syndrome with shortened limb bones and abnormal joint development (Thomas et al., Nat Genet, 12, pp. 315-317 (1996); Thomas et al., Nat Genet, 17, pp. 58-64 (1997)). Studies with transgenic mice of mis-expressed CDMP-1 suggest that this protein increases commitment of mesenchymal cells into the chondrogenic lineage (Tsumaki et al., J Cell Biol, 144, pp. 161-173 (1999)). It is anticipated that the compositions, devices and methods of this invention may be useful in the future for ameliorating these and other developmental abnormalities.

Developmental abnormalities of the bone may affect isolated or multiple regions of the skeleton or of a particular supportive or connective tissue type. These abnormalities often require complicated bone transplantation procedures and orthopedic devices. The tissue repair and regeneration required after such procedures may occur more quickly and completely with the use of a combination of at least two morphogenic proteins according to this invention. Examples of heritable conditions, including congenital bone diseases, for which use of the morphogenic compositions and devices of this invention will be useful include osteogenesis imperfecta, the Hurler and Marfan syndromes, and several disorders of epiphyseal and metaphyseal growth centers such as is presented in hypophosphatasia, a deficiency in alkaline phosphatase enzymatic activity.

Inflammatory joint diseases may also benefit from the improved morphogenic compositions and devices of this invention. These include but are not limited to infectious, non-infectious, rheumatoid and psoriatic arthritis, bursitis, ulcerative colitis, regional enteritis, Whipple's disease, and ankylosing spondylitis (also called Marie Strümpell or Bechterew's disease); the so-called “collagen diseases” such as systemic lupus erythematosus (SLE), progressive systemic sclerosis (scleroderma), polymyositis (dermatomyositis), necrotizing vasculitides, Sjögren's syndrome (sicca syndrome), rheumatic fever, amyloidosis, thrombotic thrombocytopenic purpura and relapsing polychondritis. Heritable disorders of connective tissue include Marfan's syndrome, homocystinuria, Ehlers-Danlos syndrome, osteogenesis imperfecta, alkaptonuria, pseudoxanthoma elasticum, cutis laxa, Hurler's syndrome, and myositis ossificans progressiva.

The following are examples which illustrate the morphogenic compositions and devices of this invention, and methods used to characterize them. These examples should not be construed as limiting: the examples are included for purposes of illustration and the present invention is limited only by the claims.

### EXAMPLE 1

Preparation of OP-1 from Natural Sources

For a detailed description of the procedure for purifying OP-1 from bovine bone, see Oppermann et al., U.S. Pat. No. 5,324,819, which is incorporated herein by reference.

Preparation of Demineralized Bone

Demineralized bovine bone matrix is prepared using previously published procedures (Sampath and Reddi, Proc. Natl. Acad. Sci. USA, 80, pp. 6591-95 (1983)). Fresh bovine diaphyseal bones (age 1-10 days) are stripped of muscle and fat, cleaned of periosteum, demarrowed by pressure with cold water, dipped in cold absolute ethanol, and stored at −20° C. They are then dried and fragmented by crushing and pulverized in a large mill using liquid nitrogen to prevent heating. The pulverized bone is milled to a particle size between 70-420 mm and is defatted by two washes of approximately two hours duration with three volumes of chloroform and methanol (3:1). The particulate bone is then washed with one volume of absolute ethanol and dried over one volume of anhydrous ether. Alternatively, Bovine Cortical Bone Powder (75-425 mm) may be purchased from American Biomaterials.

The defatted bone powder is demineralized with 10 volumes of 0.5 N HCl at 4° C. for 40 min., four times. Finally, neutralizing washes are done on the demineralized bone powder with a large volume of water.

Demineralized bone powder is then used as a starting material for performing the following purification steps, which are explained in detail in Oppermann et al., U.S. Pat. No. 5,324,819:
1. Dissociative extraction and ethanol precipitation;
2. Heparin-sepharose chromatography I;
3. Hydroxyapatite-ultrogel chromatography;
4. Sephacryl S-300 gel exclusion chromatography;
5. Heparin-sepharose chromatography II; and
6. Reverse phase HPLC

SDS gel electrophoresis may be performed to visualize and characterize further the species separated by HPLC; gel eluted species may be filtered, concentrated and prepared further for sequencing and other desired characterizations. The yield is typically 0.5 to 1.0 μg substantially pure osteogenic protein per kg of bone.

For additional details on these procedures and the chemical characterization of the naturally-derived osteogenic proteins, see also Oppermann et al., U.S. Pat. No. 5,258,494, which is incorporated herein by reference.

### EXAMPLE 2

Preparation of Recombinant Osteogenic Protein

A. Expression in E. Coli

Using recombinant DNA techniques, various fusion genes can be constructed to induce recombinant expression of naturally-sourced osteogenic sequences in a prokaryotic host such as E. coli. Full-length or truncated forms of the morphogenic genes encoding OP-1 or BMP-2 were cloned into a bacterial expression vector downstream from an acid labile Asp-Pro cleavage site under the control of a synthetic trp promoter-operator. Vectors were introduced into an appropriate E. coli strain by transformation and the bacteria were grown up to produce insoluble inclusion bodies.

The inclusion bodies were solubilized in 8M urea following lysis, dialyzed against 1% acetic acid, and partly purified by differential solubilization. Constructs containing the Asp-Pro site were cleaved with acid. The resulting products were passed through a Sephacryl-200HR or SP Trisacyl column to further purify the proteins, and then subjected to HPLC on a semi-prep C-18 column to separate the leader proteins and other minor impurities from the morphogenic protein constructs.

Morphogenic proteins OP-1 and BMP-2 were purified by chromatography on heparin-Sepharose. The output of the HPLC column was lyophilized at pH 2 so that it remained reduced.

Conditions for refolding were at pH 8.0 using Tris buffer and 6M guanidine-HCl at a protein concentration of several mg/ml. Those solutions were diluted with water to produce a 2M or 3M guanidine concentration and left for 18 hours at 4° C. Air dissolved or entrained in the buffer assured oxidation of the protein in these circumstances.

Samples of the various purified constructs and various mixtures of pairs of the constructs refolded together were applied to SDS polyacrylamide gels, separated by electrophoresis, sliced, incorporated in a matrix as disclosed below, and tested for osteogenic activity.

These studies demonstrated that each of the constructs (full-length or truncated versions) have true osteogenic activity. In addition, mixed species including heterodimers were also osteogenically active and may include heterodimers. For specific combinations tested, see Oppermann et al., U.S. Pat. No. 5,354,557). Finally, single and mixed species of analogs of the active region, e.g., COP5 and COP7, disclosed in U.S. Pat. No. 5,011,691, also induce osteogenesis, as determined by histological examination.

After N-terminal sequencing of the various constructs to confirm their identity, polyclonal antisera against the recombinant presumed mature form proteins were produced. The human OP-1 antisera reacted with both the glycosylated and unglycosylated higher molecular weight subunits of naturally sourced bovine material. Antisera against recombinant mature human BMP-2 reacted with both the glycosylated and unglycosylated lower molecular weight subunit of naturally sourced bovine material. While there was some cross-reactivity, this was expected in view of the significant homology between BMP-2 and OP-1 (approx. 60% identity), and the likelihood that degraded OP-1 generated during purification contaminates the lower molecular weight subunit. Both antisera react with the naturally sourced 30 ka dimeric bOP.

In addition, synthetic osteogenic sequences produced by assembly of chemically-synthesized oligonucleotides (see above) may be expressed in appropriate prokaryotic hosts. See Oppermann et al., U.S. Pat. No. 5,324,819, which is herein incorporated by reference, for an exemplary plasmid and protocol. An expression vector based on pBR322 and containing a synthetic trp promoter, operator and the modified trp LE leader can be opened at the EcoRI and PstI restriction sites, and a FB-FB COP gene fragment can be inserted between these sites, where FB is a fragment B of Staphylococcal Protein A. The expressed fusion protein results from attachment of the COP gene to a fragment encoding FB. The COP protein is joined to the leader protein via a hinge region having the sequence asp-pro-asn-gly. This hinge permits chemical cleavage of the fusion protein with dilute acid at the asp-pro site or cleavage at asn-gly with hydroxylamine. Cleavage at the hinge releases COP protein.

B. Mammalian Cell Expression

Recombinant production of mammalian proteins for therapeutic uses may be expressed in mammalian cell culture systems in order to produce a protein whose structure is most like that of the natural material. Recombinant protein production in mammalian cells requires the establishment of appropriate cells and cell lines that are easy to transfect, are capable of stably maintaining foreign DNA with an unrearranged sequence, and which have the necessary cellular components for efficient transcription, translation, post-translation modification, and secretion of the protein. In addition, a suitable vector carrying the gene of interest is necessary.

DNA vector design for transfection into mammalian cells should include appropriate sequences to promote expression of the gene of interest, including appropriate transcription initiation, termination, and enhancer sequences, as well as sequences that enhance translation efficiency, such as the Kozak consensus sequence. Preferred DNA vectors also include a marker gene and means for amplifying the copy number of the gene of interest.

Substantial progress in the development of mammalian cell expression systems has been made in the last decade and many aspects of the system are well characterized. A detailed review of the state of the art of the production of foreign proteins in mammalian cells, including useful cells, protein expression-promoting sequences, marker genes, and gene amplification methods, is disclosed in Bendig, Mary M., Genetic Engineering, 7, pp. 91-127 (1988).

Briefly, among the best characterized transcription promoters useful for expressing a foreign gene in a particular mammalian cell are the SV40 early promoter, the adenovirus promoter (AdMLP), the mouse metallothionein-I promoter (mMT-I), the Rous sarcoma virus (RSV) long terminal repeat (LTR), the mouse mammary tumor virus long terminal repeat (MMTV-LTR), and the human cytomegalovirus major intermediate-early promoter (hCMv). The DNA sequences for all of these promoters are known in the art and are available commercially.

One of the better characterized methods of gene amplification in mammalian cell systems is the use of the selectable dihydrofolate reductase (DHFR) gene in a dhfr-cell line. Generally, the DHFR gene is provided on the vector carrying the gene of interest, and addition of increasing concentrations of the cytotoxic drug methotrexate leads to amplification of the DHFR gene copy number, as well as that of the associated gene of interest. DHFR as a selectable, amplifiable marker gene in transfected chinese hamster ovary cell lines (CHO cells) is particularly well characterized in the art. Other useful amplifiable marker genes include the adenosine deaminase (ADA) and glutamine synthetase (GS) genes.

In the currently preferred expression system, gene amplification is further enhanced by modifying marker gene expression regulatory sequences (e.g., enhancer, promoter, and transcription or translation initiation sequences) to reduce the levels of marker protein produced. Lowering the level of DHFR transcription has the effect of increasing the DHFR gene copy number (and the associated OP-1 gene) in order for a transfected cell to adapt to grow in even low levels of methotrexate (MTX) (e.g., 0.1 μM MTX). Preferred expression vectors (pH754 and pH752), have been manipulated using standard recombinant DNA technology, to create a weak DHFR promoter. As will be appreciated by those skilled in the art, other useful weak promoters, different from those disclosed and preferred herein, can be constructed using standard vector construction methodologies. In addition, other, different regulatory sequences also can be modified to achieve the same effect.

The choice of cells/cell lines is also important and depends on the needs of the experimenter. Monkey kidney cells (COS) provide high levels of transient gene expression, providing a useful means for rapidly testing vector construction and the expression of cloned genes. COS cells are transfected with a simian virus 40 (SV40) vector carrying the gene of interest. The transfected COS cells eventually die, thus preventing the long term production of the desired protein product. However, transient expression does not require the time consuming process required for the development of a stable cell line.

Among established cell lines, CHO cells may be the best characterized to date, and are the currently preferred cell line for mammalian cell expression of recombinant osteogenic protein. CHO cells are capable of expressing proteins from a broad range of cell types. The general applicability of CHO cells and its successful production for a wide variety of human proteins in unrelated cell types emphasizes the underlying similarity of all mammalian cells. Thus, while the glycosylation pattern on a recombinant protein produced in a mammalian cell expression system may not be identical to the natural protein, the differences in oligosaccharide side chains are often not essential for biological activity of the expressed protein.

The methodology disclosed herein includes the use of COS cells for the rapid evaluation of vector construction and gene expression, and the use of established cell lines for long term protein production. Of the cell lines disclosed, OP-1 expression from CHO cell lines currently is most preferred.

Several different mammalian cell expression systems have been used to express recombinant morphogenic proteins which may be used together according to this invention. In particular, COS cells are used for the rapid assessment of vector construction and gene expression, using an SV40 vector to transfect the DNA sequence into COS cells. Stable cell lines are developed using CHO cells (chinese hamster ovary cells) and a temperature-sensitive strain of BSC cells (simian kidney cells, BSC40-tsA58; Biotechnology, 6, pp. 1192-96 (1988)) for the long term production of OP-1.

Two different promoters were found most useful to transcribe hOPl (SEQ. ID NO. 1): the CMV promoter and the MMTV promoter, boosted by the enhancer sequence from the Rous sarcoma virus LTR. The mMT promoter (mouse metallothionein promoter) and the SV40 late promoter have also been tested. Several selection marker genes also are used, namely, neo (neomycin) and DHFR.

The DHFR gene also may be used as part of a gene amplification scheme for CHO cells. Another gene amplification scheme relies on the temperature sensitivity (ts) of BSC40-tsA58 cells transfected with an SV40 vector. Temperature reduction to 33° C. stabilizes the ts SV40 T antigen which leads to the excision and amplification of the integrated transfected vector DNA, thereby also amplifying the associated gene of interest.

Stable cell lines were established for CHO cells as well as BSC40-tsA58 cells (hereinafter referred to as “BSC cells”). The various cells, cell lines and DNA sequences chosen for mammalian cell expression of the morphogenic proteins of this invention are well characterized in the art and are readily available. Other promoters, selectable markers, gene amplification methods and cells also may be used to express the morphogenic proteins of this invention, as well as other osteogenic proteins. Particular details of the transfection, expression, and purification of recombinant proteins are well documented in the art and are understood by those having ordinary skill in the art. Further details on the various technical aspects of each of the steps used in recombinant production of foreign genes in mammalian cell expression systems can be found in a number of texts and laboratory manuals in the art. See, e.g., F. M. Ausubel et al., ed., Current Protocols in Molecular Biology, John Wiley & Sons, New York (1989) (updated annually).

a) Exemplary Expression Vectors

Restriction maps and sources of various exemplary expression vectors designed for OP-1 expression in mammalian cells have been described (Oppermann et al., U.S. Pat. No. 5,354,557, incorporated herein by reference; see FIG. 19 (A-F) and accompanying text). Each of these vector constructs employs a full-length cDNA sequence (“hOP1”; SEQ. ID NO. 1) originally isolated from a human CDNA library (placenta) and subsequently cloned into a conventional pUC vector (pUC-18) using pUC polylinker sequences at the insertion sites.

It will be appreciated by those skilled in the art that DNA sequences encoding truncated forms of osteogenic protein may also be used in these vectors, provided that the expression vector or host cell then provides the sequences necessary to direct processing and secretion of the expressed protein.

Each vector employs an SV40 origin of replication (ori), useful for mediating plasmid replication in primate cells (e.g., COS and BSC cells). In addition, the early SV40 promoter is used to drive transcription of marker genes on the vector (e.g., neo and DHFR).

The pH717 expression vector (FIG. 19A of U.S. Pat. No. 5,354,557) contains the neomycin (neo) gene as a selection marker. This marker gene is well characterized in the art and is available commercially. Alternatively, other selectable markers may be used. The particular vector used to provide the neo gene DNA fragment for pH717 may be obtained from Clontech, Inc., Palo Alto, Calif. (pMAM-neo-blue). This vector also may be used as the backbone. In pH717, hOP1 transcription is driven by the CMV promoter with RSV-LTR (Rous sarcoma virus long terminal repeat) and MMTV-LTR (mouse mammary tumor virus long terminal repeat) enhancer sequences. These sequences are known in the art, and are available commercially. For example, vectors containing the CMV promoter sequence (e.g., pCDM8) may be obtained from Invitrogen Inc., San Diego, Calif.

Expression vector pH731 (FIG. 19B of U.S. Pat. No. 5,354,557), utilizes the SV40 late promoter to drive hOPl transcription. As indicated above, the sequence and characteristics of this promoter also are well known in the art. For example, pH731 may be generated by inserting the SmaI-BamHI fragment of hOP1 into pEUK-C1 (Clontech, Inc., Palo Alto, Calif.).

The pH752 and pH754 expression vectors contain the DHFR gene under SV40 early promoter control, as both a selection marker and as an inducible gene amplifier. The DNA sequence for DHFR is well characterized in the art, and is available commercially. For example, pH754 may be generated from pMAM-neo (Clontech, Inc., Palo Alto, Calif.) by replacing the neo gene (BamHI digest) with an SphI-BamHI, or a PvuII-BamHI fragment from pSV5-DHFR (ATCC #37148), which contains the DHFR gene under SV40 early promoter control. A BamHI site can be engineered at the SphI or PvuII site using standard techniques (e.g., by linker insertion or site-directed mutagenesis) to allow insertion of the fragment into the vector backbone. hOP1 DNA can be inserted into the polylinker site downstream from the MMTV-LTR sequence, yielding pH752 (FIG. 19D of U.S. Pat. No. 5,354,557). The CMV promoter sequence then may be inserted into pH752 (e.g., from pCDM8, Invitrogen, Inc.), yielding pH754 (FIG. 19C of U.S. Pat. No. 5,354,557).

The SV40 early promoter, which drives DHFR expression, is modified in these vectors to reduce the level of DHFR mRNA produced. Specifically, the enhancer sequences and part of the promoter sequence have been deleted, leaving only about 200 bases of the promoter sequence upstream of the DHFR gene. Host cells transfected with these vectors are adapted to grow in 0.1 pM MTX and can increase OP-1 production significantly (see, e.g., Table 8, Oppermann et al., U.S. Pat. No. 5,354,557).

The pW24 vector (FIG. 19E of U.S. Pat. No. 5,354,557), is essentially identical in sequence to p754, except that neo is used as the marker gene (see pH717) in place of DHFR. Similarly, pH783 (FIG. 19F of U.S. Pat. No. 5,354,557) contains the amplifiable marker DHFR, but here OP-1 is under mMT (mouse metallothionein promoter) control. The mMT promoter is well characterized in the art and is available commercially.

All vectors tested are stable in the various cells used to express OP-1, and provide a range of OP-1 expression levels.

b) Exemplary Mammalian Cells

Recombinant OP-1 has been expressed in three different cell expression systems: COS cells for rapidly screening the functionality of the various expression vector constructs, CHO cells for the establishment of stable cell lines, and BSC40-tsA58 cells as an alternative means of producing OP-1 protein. The CHO cell expression system disclosed herein is contemplated to be the best mode currently known for long-term recombinant OP-1 production in mammalian cells.

(1) COS Cells

COS cells (simian kidney cells) are used for rapid screening of vector constructs and for immediate, small scale production of OP-1 protein. COS cells are well known in the art and are available commercially. The particular cell line described herein may be obtained through the American Type Culture Collection (ATCC #COS-1, CRL-1650).

OP-1 expression levels from these different expression vectors, analyzed by Northern and Western blot assays, are compared Oppermann et al. (see Table 7, Oppermann et al.).

Large scale preparations of OP-1 from transfected COS cells may be produced using conventional roller bottle technology. Briefly, 14×10⁶ cells are used to seed each bottle. After 24 hrs of growth, the cells are transfected with 10 μg of vector DNA (e.g., pH717) per 10⁶ cells, using the DEAE-dextran method. Cells are then conditioned in serum-free media for 120 hr before harvesting the media for protein analysis. Following this protocol, OP-1 yield is approximately 2-6 ng/ml.

(2) BSC Cells

The BSC40-tsA58 cell line (“BSC cells”) is a temperature-sensitive (ts) strain of simian kidney cells (Biotechnology, 6, pp. 1192-96 (1988)) which overcomes some of the problems associated with COS cells. These BSC cells have the advantage of being able to amplify gene sequences rapidly on a large scale with temperature downshift, without requiring the addition of exogenous, potentially toxic drugs. In addition, after induction and stimulation of OP-1 expression, the cells may be transferred to new growth medium, grown to confluence at 39.5° C. and induced a second time by downshifting the temperature to 33° C. BSC cells may be used to establish stable cell lines rapidly for protein production.

OP-1 expression in transfected BSC cells may be induced by shifting the temperature down to 33° C. in media containing 10% FCS, and harvesting the conditioned media after 96 hrs of incubation. Comparable amounts of OP-1 mRNA and protein are obtained, as compared with CHO cells (e.g., 100-150 ng OP-1/ml conditioned media from BSC clones transfected with pH717, see Oppermann et al.).

(3) CHO Cells

CHO cells (chinese hamster ovary cells) may be used for long term OP-1 production and are the currently preferred cell line for mammalian cell expression of OP-1 CHO cell lines are well characterized for the small and large scale production of foreign genes and are available commercially. See Oppermann et al., U.S. Pat. No. 5,354,557, incorporated herein by reference, for a detailed description of: establishing a stable transfected cell line with high hOP-1 expression levels, subcloning transfected cells to obtain high expression subclones, characterizing subclone DNA insert copy numbers, and screening subclones for OP-1 mRNA and protein expression levels. Oppermann et al. also provides a detailed description of a rapid purification method for obtaining recombinantly produced OP-1 of about 90% purity, and further data demonstrating the physical characteristics (molecular weight and glycosylation profiles) and osteogenic activities of a variety of recombinant forms of OP-1 expressed in the cell lines described above.

Accordingly, it is anticipated that active mature OP-1 sequences, including full-length, truncated and mutationally-altered active forms of the protein, can be expressed from other different prokaryotic and eukaryotic cell expression systems using procedures essentially as described herein. The proteins produced may have varying N-termini, and those expressed from eukaryotic cells may have varying glycosylation patterns. Finally, it will also be appreciated that these variations in the recombinant osteogenic protein produced will be characteristic of the host cell expression system used rather than of the protein itself.

### EXAMPLE 3

Synergistic Effect of Exogenous CDMPs on the OP-1-induced Alkaline Phosphatase Activity in C2C12 Cells

A. Cell Culture

Primary pluripotent mesenchymal precursor cells (originally purchased from American Type Culture Collection, Rockville, Md.) were cultured in DMEM (Life Technologies, Grand Island, N.Y.) containing 10% FBS and 100 units/mL of penicillin/100 mg/mL of streptomycin (Life Technologies, Grand Island, N.Y.) at 37° C. in a humidified 5% CO₂ atmosphere. Media were replenished every 3 days.

For measurement of alkaline phosphatase (AP) activity, cells were grown in 48-well plates in DMEM containing 5% FBS in the absence or presence of OP-1, CDMP-1, -2, and -3 alone and the combination of a fixed concentration of OP-1 and two different concentrations of each CDMP. At the end of the treatment period, the cultures were rinsed with PBS and stained for AP activity using a commercial kit (Sigma, St. Louis, Mo.). For isolation of total RNA, cells were grown in 100-mm culture dishes.

B. Alkaline Phosphatase Activity Assay

At the end of the indicated culture periods, alkaline phosphatase enzymatic activity was measured in cell lysates. Cell lysates were prepared by aspirating the medium from the 48-well plate, rinsing the cells with ice-cold PBS, and sonicating them with 0.1% Triton X-100 in PBS (100 ml/well) for 5 min at room temperature. Alkaline phosphatase activity in the lysates was measured in 2-amino-2-methyl-l-propanol buffer (pH 10.3) with p-nitrophenyl phosphate as substrate at 37° C. Reactions were performed in 96-well plates for 1-2 h. The total cellular AP activity was measured with p-nitrophenyl phosphate as a substrate in 2-amino-2-methyl-1-propanol buffer, pH 10.3, at 37° C. using a commercial assay kit (Sigma Chemical Co., St. Louis, Mo.). Following color development, reactions were terminated by the addition of 0.5 N NaOH. Absorbance of the reaction mixture was measured at 405 nm using a Hewlett Packard Genenchem automatic plate reader (Hewlett-Packard, Palo Alto, Calif.). Total protein level in the lysates was measured according to Bradford (M. Bradford, Anal. Biochem., 72, pp.248-54 (1976)) using bovine serum albumin as a standard. Alkaline phosphatase activity was expressed as nmol p-nitrophenol liberated per microgram of total cellular protein.

Previous studies have shown that OP-1 alone stimulated a dose-dependent increase in AP activity in C2C12 cells (Yeh et al., J. Cell. Biochem., 87, pp. 292-304 (2002). An alkaline phosphatase (AP) assay was performed as previously described to test the effects of increasing exogenous CDMPs in combination with a single concentration (100 ng/ml) of osteogenic protein OP-1.

At least four experimental groups were tested: control cells treated with no OP-1 or CDMP (CON); group I cells treated with increasing concentrations of CDMP alone; group II cells treated with 100 ng/ml of OP-1 alone; and group III cells, treated with 100 ng/ml OP-1 in the presence of increasing concentrations of the three different CDMPs.

FIG. 1 shows the effects of CDMP-1 (50-200 ng/ml dissolved in 4.75% ethanol/0.01% trifluoroacetic acid; provided by Stryker Biotech (Hopkinton, Mass.)) and 100 ng/ml of OP-1 on C2C12 cell alkaline phosphatase activity at 5 and 7 days post-treatment. OP-1 (100 ng/ml) alone stimulated AP activity by about four-fold (p<0.001) and twenty-three-fold (p<0.001) after 5 and 7 days, respectively. CDMP-1 (200 ng/ml) alone stimulated a two-and four-fold (p<0.002) increase in AP activity after 5 and 7 days, respectively. Exogenous CDMP-1 enhanced the OP-1 stimulated AP activity in a dose-dependent manner. Maximum enhancements of about sixteen- and eighty-five-fold (p<0.001) compared with the solvent-treated control value were observed at 100 ng/ml OP-1 and 200 ng/ml CDMP-1 after 5 and 7 days, respectively. Under these conditions, the AP activity was enhanced by about four-fold (p<0.001) compared with that induced by OP-1 alone for both treatment periods.

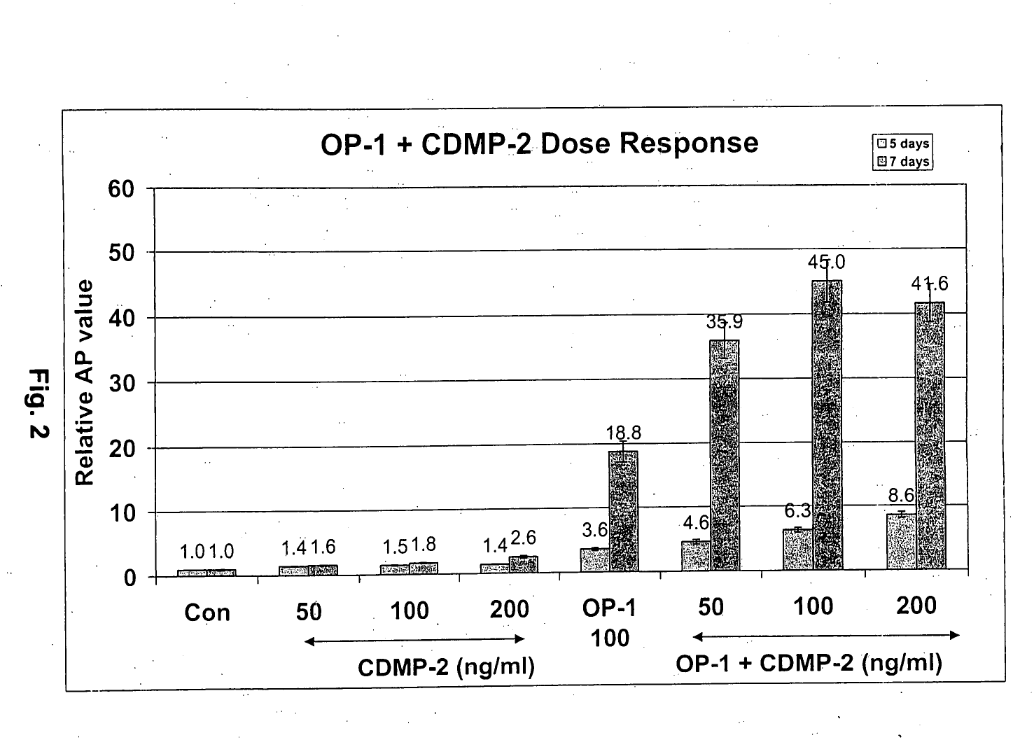

FIG. 2 shows the effects of CDMP-2 (50-200 ng/ml dissolved in 4.75% ethanol/0.01% trifluoroacetic acid; provided by Stryker Biotech (Hopkinton, Mass.)) and 100 ng/ml of OP-1 on C2C12 cell alkaline phosphatase activity at 5 and 7 days post-treatment. After 5 days of treatment, exogenous CDMP-2 (from 50 ng/ml up to 200 ng/ml) alone did not stimulate AP activity significantly. After 7 days of treatment, CDMP-2 at 200 ng/ml alone stimulated AP activity by about three-fold (p<0.005), and enhanced the OP-1-stimulated AP activity in a dose-dependent manner. A maximum enhancement was observed at 100 ng/ml OP-1 and 100 ng/ml CDMP-2. Maximum enhancements of about nine- and forty- to forty-five-fold (p<0.001) compared with the solvent-treated control value were observed after 5 and 7 days, respectively. Under these conditions, the AP activity was stimulated by approximately two-fold (p<0.001) compared with that induced by OP-1 alone for both treatment durations.

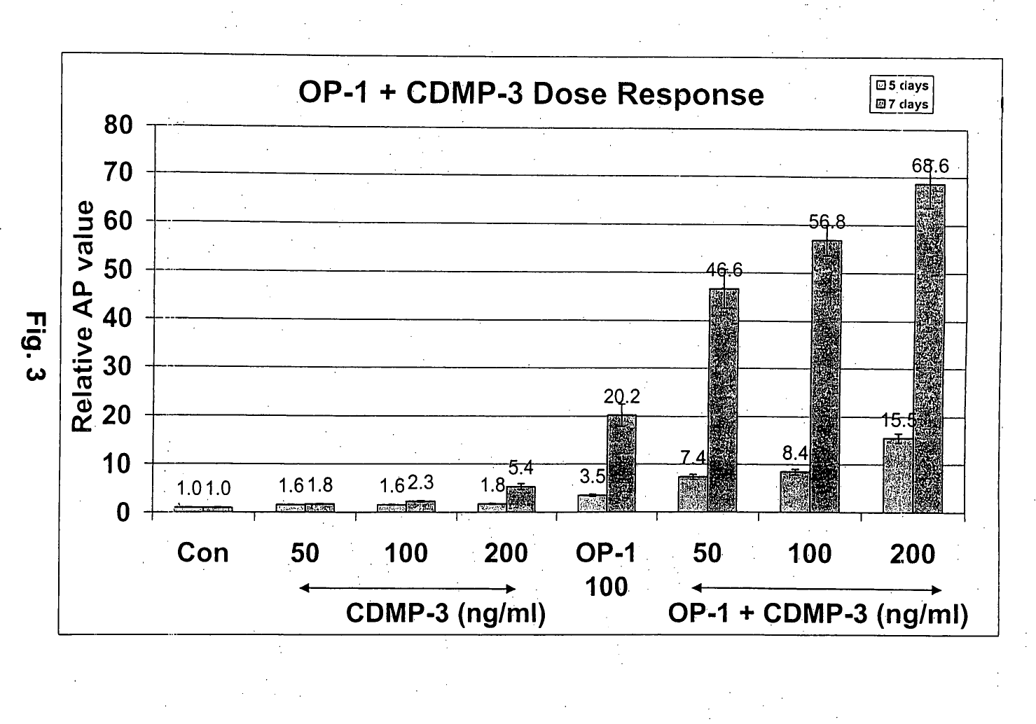

FIG. 3 shows the effects of CDMP-3 (50-200 ng/ml dissolved in 4.75% ethanol/0.01% trifluoroacetic acid; provided by Stryker Biotech (Hopkinton, Mass.)) and 100 ng/ml of OP-1 on C2C12 cell alkaline phosphatase activity at 5 and 7 days post-treatment. After 5 and 7 days of treatment, exogenous CDMP-3 (200 ng/ml) alone stimulated AP activity by about two-fold (p<0.005) and about five-fold (P<0.002), respectively. After 7 days, CDMP-3 alone further enhanced the OP-1-stimulated AP activity in a dose-dependent manner. A maximum enhancement was observed at 100 ng/ml OP-1 and 200 ng/ml CDMP-3. Maximum enhancements of about sixteen- and sixty-nine-fold (p<0.001) compared with the solvent-treated control value were observed after 5 and 7 days, respectively. Under these conditions, the AP activity was elevated by approximately four-fold (p<0.001) above the levels produced by OP-1 alone for both treatment periods.

C. Statistical Analysis

Data were presented as the mean±SEM. Statistical differences between means were determined by one-way ANOVA, followed by post-hoc Least Significant Difference Multiple Comparisons in the SIMSTAT3 software package (Normand Peladau, Provalis Research, Montréal, Canada).

These results confirm that not only is OP-1 alone capable of inducing osteoblastic cell differentiation of the pluripotent mesenchymal cell line C2C12 (Yeh et al., J. Cell Biochem., 87, pp. 292-304 (2002)), but that induction of OP-1 was significantly and synergistically stimulated by CDMP-1, CDMP-2 and CDMP-3. All three CDMPs enhanced significantly the OP-1 stimulated AP activity in C2C12 cells. At 200 ng/ml CDMP-1 is most potent in enhancing AP activity, CDMP-3 is about 80% as potent as CDMP-1, and CDMP-2 is the least potent (50%).

### EXAMPLE 4

Synergistic Effect of Exogenous CDMPs on the OP-1-induced Alkaline Phosphatase Staining in C2C12 Cells

A. Alkaline Phosphatase Staining Assay

C2C12 cells grown in 12-well plates, were cultured in the absence or presence of OP-1, CDMP-1, -2, and -3 alone and the combination of a fixed concentration of OP-1 and two different concentrations of each CDMP. After 5 days, the cultures were rinsed with PBS and stained for alkaline phosphatase activity using a commercial kit (Sigma , St. Louis, Mo.).

The enhancement of the OP-1 induced AP activity by CDMPs may be due to an increase in either the number of AP-positive cells or the AP level in the OP-1-responsive cells without increasing the number of responsive cells. To distinguish these two possibilities, equal numbers of C2C12 cells were plated and treated with two different concentrations of CDMPs in the presence or absence of OP-1 (100 ng/ml).

Briefly, C2C12 cell cultures were prepared using published procedures (Yeh et al., J. Cell. Biochem., 87, pp. 292-304 (2002)). Briefly, cells were harvested by brief trypsination followed by inactivation of the trypsin activity by serum (10%), and subsequent repeated washing of the detached cells with HBSS. C2C12 cells were plated in complete medium (MEMO, alpha; GIBCO/BRL, Grand Island, N.Y.) containing 10% fetal bovine serum, vitamin C (100 μg/ml), and antibiotics (100 U/ml penicillin, and 100 mg/ml streptomycin). Cultures were incubated at 37° C. with 95% air/5% CO₂ for several days to reach confluence. Cells were then subcultured in 48-well plates (Corning, Acton, Mass.) for AP activity measurements in complete MEM medium with 10% fetal bovine serum for experimentation.

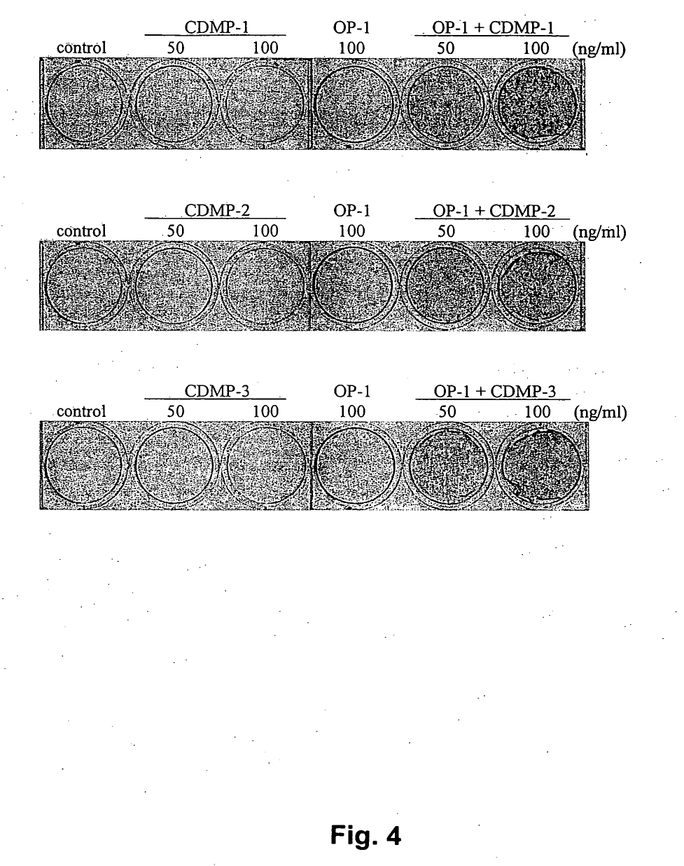

FIG. 4 shows the effect of two different concentrations (50 and 100 ng/ml) of CDMP-1 (FIG. 4A), CDMP-2 (FIG. 4B), and CDMP-3 (FIG. 4C) in the presence or absence of OP-1 (100 ng/ml) on C2C12 cell alkaline phosphatase staining at 7 days post-treatment. After 7 days, the cultures were rinsed with PBS and stained for alkaline phosphatase activity using a commercial cytochemical kit (Sigma Chemical Co., St. Louis, Mo.). The intensity of the blue stain is proportional to the AP activity. Images of stained cells were captured using an Olympus CK2 inverted microscope equipped with a CCD camera Model 300-RC (DAGE/MTI, Michigan Cit, Ind.).

AP-positive cells were detected in cultures treated with solvent control or any one of CDMP-l, -2 or -3 in the absence of OP-1. However, OP-1 alone increased the number of AP-positive cells. The number of AP-positive cells in cultures treated with the combination of CDMP and OP-1 appeared to be similar to that treated with OP-1 alone (183±15 vs. 192±14 AP-positive cells/field). However, cultures treated with the combination of OP-1 and CDMPs showed dramatically stronger AP staining intensity than those treated with OP-1 or CDMP alone. The increase in staining intensity was also dependent on the CDMP concentration.

The induction by OP-1 was significantly and synergistically stimulated by CDMP-1, -2, and -3. The AP staining data on C2C12 cells treated with the combination of OP-1 and CDMP suggest that the combination enhanced OP-1 action by stimulating the same cells that responded to OP-1 and not by activating additional cells.

### EXAMPLE 5

Effect of Exogenous CDMPs on the osteocalcin, MyoD and scleraxis mRNA expression in C2C12 Cells

A. RNA Isolation

Confluent C2C12 cells in 100-mm culture dishes were grown in the presence of solvent vehicle (control), CDMP-1, -2 and -3 alone (200 ng/ml), OP-1 alone (100 ng/ml) or the combination of 100 ng/ml OP-1 and 200 ng/ml CDMPs for 5 and 7 days. At the end of treatment interval, cells in the 100-mm culture dishes were rinsed with ice-cold 1×PBS solution to remove DMEM media. Total RNA was isolated using the TRI reagent from Sigma (St. Louis, Mo.) following the manufacturer's instructions. RNA was dissolved in diethyl pyrocarbonate-treated H₂O, and the concentration of RNA was measured by its absorbance at 260 nm. The intactness of the RNA sample was examined by gel electrophoresis on 1% agarose after ethidium bromide staining. Only RNA preparations showing intact species were used for subsequent analyses.

Total RNA was isolated with cold Utraspec (Biotecx Lab., Houston, Tex.) following the manufacturer's recommendation. RNA was recovered by precipitation and dissolved in DEPC-H₂O. The amount of RNA recovered was estimated by A₂₆₀ reading. The integrity of the RNA preparation was examined by gel electrophoresis on 1% agarose. RNA was detected by EtBr staining. Only RNA preparations showing intact species were used for subsequent analyses.

B. Northern Blot Analysis

Total RNAs (20 μg) were denatured with formaldehyde and formamide at 65° C. for 15 min and analyzed on a 1% GTG agarose gel containing 2.2 M formaldehyde. RNA standards (0.24-9.5 kb) from GIBCO/BRL (Grand Island, N.Y.) were used as size markers. The fractionated RNA was transferred onto “Nytran Plus” membrane using a Turboblot apparatus (Schleicher & Schuell, Inc., Keene, N.H.). The lane containing the standards was removed from the blot and stained with methylene blue. The RNA was covalently linked to the membrane using a UV Crosslinker (Stratagene, La Jolla, Calif.). The membranes were hybridized overnight at 42° C. with the cDNA probes. All cDNA fragments used for Northern blot analyses were produced by digestion of the parent plasmids with the appropriate pairs of restriction endonucleases. The resultant DNA fragments were purified by agarose gel electrophoresis and Geneclean II (BIO 101, La Jolla, Calif.). The 440-bp Myo-D probe was obtained by digestion of pT7T3D-Pac (ATCC Clone ID 1064620) with PstI. The 520-bp osteocalcin (OC) probe was obtained by digestion of pOC/BS plasmid with EcoRI restriction enzyme. The plasmid pOC/BS was constructed by ligation of a 520-bp DNA containing the entire coding sequence of the rat osteocalcin gene and pBS (Clonetech, Palo Alto, Calif.). The 410-bp scleraxis probe was obtained by digestion of the PGEM-T Easy-Scx plasmid with SacI/BalI restriction enzyme. The pGEM-T Easy-Scx plasmid was constructed by ligation of a 1.5 kb PCR-generated DNA fragment containing the scleraxis gene and the pGEM-T Easy plasmid (Promega, Madison, Wis.). Purified cDNA fragments were labeled with ³²P-deoxy-ATP using the Strip-EZ DNA labeling system (Ambion Co., Austin, Tex.). The labeled cDNA probes were purified through a Midi-SELECT G-25 spin column (5 Prime-3 Prime, Boulder, Colo.) to remove the unincorporated nucleotides. The 18S ribosomal RNA (rRNA) was probed with a ³²P-labeled 18S-specific oligonucleotide (5′-GCCGTGCGTACTTAGACATGCATG-3′) (SEQ ID NO:10).

Following incubation with a particular cDNA probe, membranes were washed twice in 2×SSC at room temperature for 20 min each, twice in 2×SSC/1% SDS at 60° C. for 1 hour each, and finally twice in 0.1×SSC at room temperature for 30 min each. The blots were exposed to a PhosphorImage screen and the intensity of the signal was quantified using the ImageQuant Software from Molecular Dynamics (Sunnyvale, Calif.). Before probing with another CDNA probe, the signal from the previous probe was stripped from the blot using Ambion's Strip-EZ Degradation and Reconstitution buffers following the manufacturer's recommendation (Ambion, Austin, Tex.). The blots were also probed with an 18S rRNA oligonucleotide probe to correct for loading variations.

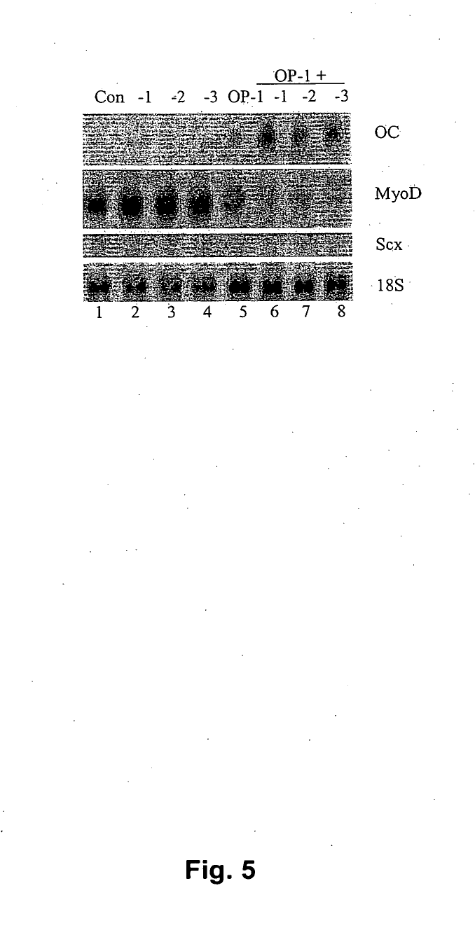

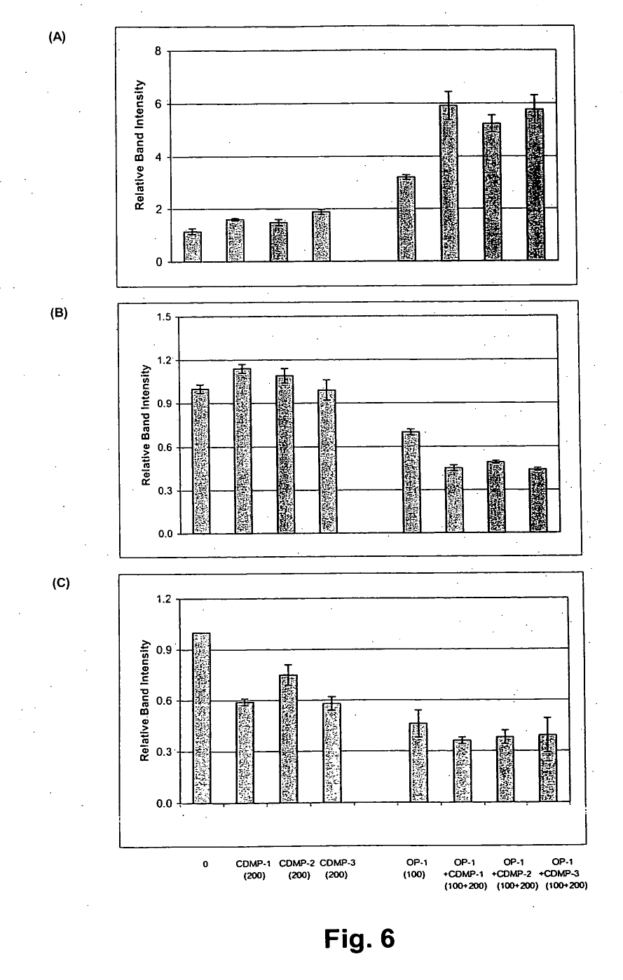

FIG. 5 shows a representative Northern blot for osteocalcin (OC), MyoD and scleraxis mRNA. FIG. 6 shows the qualitative analysis of the intensity of the hybridized RNA species on the Northern blots described in FIG. 5, specifically. Osteocalcin mRNA was not detectable in control cultures after 5 days of treatment, and its level was elevated by approximately 1.5 to 1.8-fold (p<0.005) in cultures treated with CDMP-1, -2, and -3 compared to that in control cells. By contrast, the osteocalcin mRNA level in OP-1-treated cultures increased by about three-fold (p<0.001). In cells treated with the combination of 100 ng/ml OP-1 and 200 ng/ml CDMP-1, -2, or -3, the MRNA levels increased further by approximately six-, five- and six-fold, respectively, compared with the control cells (see FIG. 6A). Under these conditions, the enhancements were about two-fold above the levels produced by OP-1 alone. This results show that the combined OP-1 and CDMP treatment of C2C12 cells produced a greater stimulation of osteocalcin mRNA expression than OP-1 alone. All three CDMPs enhanced significantly the OP-1 stimulated osteocalcin mRNA level in C2C112 cells.

Previous studies have indicated that OP-1 inhibited the MRNA expression of regulator factors of myogenic differentiation (Yeh et al., J. Cell Biochem., 87:292-304 (2002). FIG. 5 shows that MyoD mRNA was not altered in cultures treated with CDMPs alone, but decreased to about 70% of the control in cultures treated with OP-1 alone (see FIG. 6B). The mRNA level was further decreased by treatment with the combination of OP-1 and CDMP-1, -2, and -3. The combination treatments decreased the MyoD mRNA level to about 40 to 50% of the control (see FIG. 6B). The results further demonstrate that OP-1, but not the CDMPs alone, down-regulated mRNA expression of the muscle-specific regulatory factor MyoD and that CDMPs enhanced the OP-1-induced down-regulation of MyoD mRNA expression. These results demonstrate that OP-1 inhibits myogenic differentiation and converts the differentiation pathway of C2C12 cells into that of the osteoblast lineage and that the CDMPs synergistically enhanced the OP-1 action.

Scleraxis is a novel class II helix-loop-helix transcription factor which is constitutively expressed in C2C112 cell culture (McLellan et al., Gene Expression Patterns, 2: 329-335 (2002). Scleraxis was shown to be essential for mesodermal development and was found to be a specific marker for developing tendons and ligaments (Schweitzer et al., Development, 128: 3855-3866 (2001). The mRNA expression of scleraxis in C2C112 cell cultures treated with OP-1 in the presence of varying concentrations of CDMPs was examined. FIG. 5 shows that OP-1 and all three CDMPs tested lowered the mRNA expression of scleraxis in C2C112 cells. The present study showed that OP-1, CDMP-1, -2, and -3 individually or in combinations synergistically suppressed the scleraxis mRNA expression in C2C12 cells (see FIG. 6C) and hence, suppressed the stimulation of this cell line to form tendon/ligament tissue.

### EXAMPLE 6

Effect of Exogenous CDMPs on mRNA Expression of Members of the BMP family and their Receptors

To determine whether the combination of OP-1 and CDMP altered the expression levels of other BMPs, the mRNA expression levels of BMPs were measured by RNase Protection Assay (RPA).

The RiboQuant RPA kits with the mBMP-1, the mGDF-1 and the MBMPR Multi-Probe Template Sets were purchased from BD PharMingen (San Diego, Calif.) and used according to the manufacturer's instructions. The mBMP-1 kit allows detection of mRNAs for BMP-1, -2, -3, -4, -5, -6, -7, -8A and -8B with the protected fragment of 148, 160, 181, 226, 253, 283, 316, 353, and 133 nucleotides in length, respectively. The MBMPR kit allows detection of ALK-1, ALK-2 (ActR-I), ALK-3 (BMPR-IA), ALK-4, ALK-5, ALK-6 (BMPR-IB), ALK-7, AVR-2 (ActR-II), AVR2B (ActR-IIB), and MIS2R with the protected fragments of 430, 388, 349, 313, 280, 250, 223, 199, 178 and 161 nucleotides in length, respectively. All three kits also detect mRNA for ribosomal protein L32 and GAPDH as controls, allowing for correcting sampling or technique errors. The protected RNA fragments were separated on 8M urea/5% polyacrylamide gels. After electrophoresis, gels were fixed and dried. Radioactive bands were detected using the PhosphorImager and their intensities were quantified with the ImageQuant Software (Molecular Dynamics, Sunnyvale, Calif.).

The expression levels of BMP-1, -4, -5, -6, and -8 as well as GDF-1, -5, -6, -8, -9 in cultures treated with the combination were not significantly changed compared to those treated with OP-1, CDMP-1, -2 or -3 alone. RPA was also used to measure the mRNA expression levels of 10 different BMP receptors. ActR-I, BMPR-IA, BMPR-IB and ALK-7 mRNAs were detected by the kit and their levels of expression were not significantly changed compared to those treated with OP-1, CDMP-1, -2, or -3 alone.

The observation that the mRNA expression levels of BMP and BMPR were not altered further in cell cultures treated with the combination of CDMPs and OP-1 compared to those treated by individual protein alone indicate the synergistic action is not by stimulating the expression of BMPs and BMPRs.

### EXAMPLE 7

Preparation of Bone-Derived Matrices for Use In Morphogenic Devices

Demineralized bone matrix, preferably bovine bone matrix, is prepared using previously published procedures (Sampath and Reddi, Proc. Natl. Acad. Sci. USA, 80, pp. 6591-95 (1983)), as described in Example 1.

Demineralized bone matrix is extracted with 5 volumes of 4M guanidine-HCl, 50 mM Tris-HCl, pH 7.0 for 16 hr. at 4° C. The suspension is filtered. The insoluble material is collected and used to fabricate the matrix. The material is mostly collagenous in nature and is devoid of osteogenic or chondrogenic activity.

The major component of all bone matrices is Type-I collagen. In addition to collagen, demineralized bone extracted includes non-collagenous proteins which may account for 5% of its mass. In a xenogenic matrix, these non-collagenous components may present themselves as potent antigens, and may constitute immunogenic and/or inhibitory components. These components also may inhibit osteogenesis in allogenic implants by interfering with the developmental cascade of bone differentiation.

Treatment of the matrix particles with a collagen fibril-modifying agent extracts potentially unwanted components from the matrix, and alters the surface structure of the matrix material. Useful agents include acids, organic solvents or heated aqueous media. Various treatments are described below. A detailed physical analysis of the effect these fibril-modifying agents have on demineralized, quanidine-extracted bone collagen particles is disclosed in U.S. Pat. No. 5,171,574, the disclosure of which is hereby incorporated by reference.

After contact with the fibril-modifying agent, the treated matrix is washed to remove any extracted components, following a form of the procedure set forth below:
1. Suspend in TBS (Tris-buffered saline) 1 g/200 ml and stir at 4° C. for 2 hrs; or in 6M urea, 50 mM Tris-HCl, 500 mM NaCl, pH 7.0 (UTBS) or water and stir at room temperature (RT) for 30 minutes (sufficient time to neutralize the pH);
2. Centrifuge and repeat wash step; and
3. Centrifuge; discard supernatant; wash residue with water; and lyophilize.
   Acid Treatments
   1. Trifluoroacetic Acid

Trifluoroacetic acid is a strong non-oxidizing acid that is a known swelling agent for proteins, and which modifies collagen fibrils.

Bovine bone residue prepared as described above is sieved, and particles of the appropriate size are collected. These particles are extracted with various percentages (1.0% to 100%) of trifluoroacetic acid and water (v/v) at 0° C. or at room temperature for 1-2 hours with constant stirring. The treated matrix is filtered, lyophilized, or washed with water/salt and then lyophilized.

2. Hydrogen Fluoride

Like trifluoroacetic acid, hydrogen fluoride (HF) is a strong acid and swelling agent, and also is capable of altering intraparticle surface structure. Hydrogen fluoride is also a known deglycosylating agent. As such, HF may function to increase the osteogenic activity of these matrices by removing the antigenic carbohydrate content of any glycoproteins still associated with the matrix after guanidine extraction.

Bovine bone residue prepared as described above is sieved, and particles of the appropriate size are collected. The sample is dried in vacuo over P₂O₅, transferred to the reaction vessel and exposed to anhydrous hydrogen fluoride (10-20 ml/g of matrix) by distillation onto the sample at -70° C. The vessel is allowed to warm to 0° C. and the reaction mixture is stirred at this temperature for two hours. After evaporation of the hydrogen fluoride in vacuo, the residue is dried thoroughly in vacuo over KOH pellets to remove any remaining traces of acid. Extent of deglycosylation can be determined from carbohydrate analysis of matrix samples taken before and after treatment with hydrogen fluoride, after washing the samples appropriately to remove non-covalently bound carbohydrates. SDS-extracted protein from HF-treated material is negative for carbohydrate as determined by Con A blotting.

The deglycosylated bone matrix is next washed twice in TBS (Tris-buffered saline) or UTBS, water-washed, and then lyophilized.

Other acid treatments are envisioned in addition to HF and TFA. TFA is a currently preferred acidifying reagent in these treatments because of its volatility. However, it is understood that other, potentially less caustic acids may be used, such as acetic or formic acid.

Solvent Treatments

1. Dichloromethane

Dichloromethane (DCM) is an organic solvent capable of denaturing proteins without affecting their primary structure. This swelling agent is a common reagent in automated peptide synthesis, and is used in washing steps to remove components. Bovine bone residue, prepared as described above, is sieved, and particles of the appropriate size are incubated in 100% DCM or, preferably, 99.9% DCM/0.1% TFA. The matrix is incubated with the swelling agent for one or two hours at 0° C. or at room temperature. Alternatively, the matrix is treated with the agent at least three times with short washes (20 minutes each) with no incubation.

2. Acetonitrile

Acetonitrile (ACN) is an organic solvent capable of denaturing proteins without affecting their primary structure. It is a common reagent used in high-performance liquid chromatography, and is used to elute proteins from silica-based columns by perturbing hydrophobic interactions.

Bovine bone residue particles of the appropriate size, prepared as described above, are treated with 100% ACN (1.0 g/30 ml) or, preferably, 99.9% ACN/0.1% TFA at room temperature for 1-2 hours with constant stirring. The treated matrix is then water-washed, or washed with urea buffer or 4M NaCl, and lyophilized. Alternatively, the ACN or ACN/TFA treated matrix may be lyophilized without wash.

3. Isopropanol

Isopropanol is also an organic solvent capable of denaturing proteins without affecting their primary structure. It is a common reagent used to elute proteins from silica HPLC columns. Bovine bone residue particles of the appropriate size prepared as described above are treated with 100% isopropanol (1.0 g/30 ml) or, preferably, in the presence of 0.1% TFA, at room temperature for 1-2 hours with constant stirring. The matrix is then water-washed or washed with urea buffer or 4M NaCl before being lyophilized.

4. Chloroform

Chloroform also may be used to increase surface area of bone matrix like the reagents set forth above, either alone or acidified. Treatment as described above is effective to assure that the material is free of pathogens prior to implantation.

Heat Treatment

The currently most preferred agent is a heated aqueous fibril-modifying medium such as water, to increase the matrix particle surface area and porosity. The currently most preferred aqueous medium is an acidic aqueous medium having a pH of less than about 4.5, e.g., within the range of about pH 2-pH 4 which may help to “swell” the collagen before heating. Acetic acid (0.1%), which has a pH of about 3, currently is most preferred. 0.1M acetic acid also may be used.

Various amounts of delipidated, demineralized guanidine-extracted bone collagen are heated in the aqueous medium (1 g matrix/30 ml aqueous medium) under constant stirring in a water jacketed glass flask, and maintained at a given temperature for a predetermined period of time. Preferred treatment times are about one hour, although exposure times of between about 0.5 to two hours appear acceptable. The temperature employed is held constant at a temperature within the range of about 37° C. to 65° C. The currently preferred heat treatment temperature is within the range of about 45° C. to 60° C.

After the heat treatment, the matrix is filtered, washed, lyophilized and used for implantation. Where an acidic aqueous medium is used, the matrix also is preferably neutralized prior to washing and lyophilization. A currently preferred neutralization buffer is a 200 mM sodium phosphate buffer, pH 7.0. To neutralize the matrix, the matrix preferably is first allowed to cool following thermal treatment, the acidic aqueous medium (e.g., 0.1% acetic acid) is then removed and replaced with the neutralization buffer and the matrix agitated for about 30 minutes. The neutralization buffer may then be removed and the matrix washed and lyophilized (see infra).

The effects of heat treatment on morphology of the matrix material is described in Oppermann, et. al., U.S. Pat. No. 5,354,557. Hot aqueous treatment can increase the degree of micropitting on the particle surface (e.g., about 10-fold,) as well as also substantially increasing the particle's porosity. This alteration of the matrix particle's morphology substantially increases the particle surface area. Careful measurement of the pore and micropit sizes reveals that hot aqueous medium treatment of the matrix particles yields particle pore and micropit diameters within the range of 1 μm to 100 μm.

Oppermann et al. also show that a complete solvent extract from hot water-treated matrix inhibits OP-1 induced new bone formation in a dose dependent manner. Thus such treatment may also be removing component(s) whose association with the matrix may interfere with new bone formation in vivo.

The matrix also may be treated to remove contaminating heavy metals, such as by exposing the matrix to a metal ion chelator. For example, following thermal treatment with 0.1% acetic acid, the matrix may be neutralized in a neutralization buffer containing sodium EDTA, e.g., 200 mM sodium phosphate, 5 mM EDTA, pH 7.0. The use of 5 mM EDTA provides about a 100-fold molar excess of chelator to residual heavy metals present in the most contaminated matrix tested to date. Subsequent washing of the matrix following neutralization appears to remove the bulk of the EDTA. EDTA treatment of matrix particles reduces the residual heavy metal content of all metals tested (Sb, As, Be, Cd, Cr, Cu, Co, Pb, Hg, Ni, Se, Ag, Zn, Tl) to less than about 1 ppm. Bioassays with EDTA-treated matrices indicate that treatment with the metal ion chelator does not inhibit bone inducing activity.

The collagen matrix materials preferably take the form of a fine powder, insoluble in water, comprising nonadherent particles. It may be used simply by packing into the volume where new bone growth or sustained release is desired, held in place by surrounding tissue. Alternatively, the powder may be encapsulated in, e.g., a gelatin or polylactic acid coating, which is absorbed readily by the body. The powder may be shaped to a volume of given dimensions and held in that shape by interadhering the particles using, for example, soluble, species- biocompatible collagen. The material may also be produced in sheet, rod, bead, or other macroscopic shapes.

Demineralized rat bone matrix used as an allogenic matrix may be prepared from several of the dehydrated diaphyseal shafts of rat femur and tibia (as described in Oppermann et al., U.S. Pat. No. 5,354,557, which is incorporated herein by reference) to produce a bone particle size that passes through a 420 μm sieve. The bone particles are subjected to dissociative extraction with 4M guanidine-HCl. Such treatment results in a complete loss of the inherent ability of the bone matrix to induce endochondral bone differentiation. The remaining insoluble material is used to fabricate the matrix. The material is mostly collagenous in nature, and upon implantation, does not induce cartilage and bone formation. All new preparations are tested for mineral content and osteogenic activity before use. The total loss of biological activity of bone matrix is restored when an active morphogenic protein fraction or a substantially pure morphogenic protein preparation is reconstituted with the biologically inactive insoluble collagenous matrix.

Ethanol Trifluoroacetic Acid Lyophilization

In this procedure, morphogenic protein is solubilized in an ethanol-trifluoroacetic acid solution (47.5% EtOH/0.01% TFA) and added to the carrier material. Samples are vortexed and then lyophilized. This method is currently preferred.

Acetonitrile Trifluoroacetic Acid Lyophilization

This is a variation of the above procedure, using an acetonitrile-trifluoroacetic acid (ACN/TFA) solution to solubilize the morphogenic proteins that are then added to the carrier material. Samples are vigorously vortexed many times and then lyophilized.

Ethanol Precipitation

Matrix is added to morphogenic protein dissolved in guanidine-HCl. Samples are vortexed and incubated at a low temperature (e.g., 4° C.). Samples are then further vortexed. Cold absolute ethanol (5 volumes) is added to the mixture which is then stirred and incubated, preferably for 30 minutes at −20° C. After centrifugation (microfuge, high speed), the supernatant is discarded. The reconstituted matrix is washed twice with cold concentrated ethanol in water (85% EtOH) and then lyophilized.

Urea Lyophilization

For those morphogenic proteins that are prepared in urea buffer, the combination of morphogenic protein are mixed together with the matrix material, gently vortexed and then lyophilized. The lyophilized material may be used “as is” for implants.

Buffered Saline Lyophilization

The combination of morphogenic proteins in physiological saline may also be vortexed with the matrix and lyophilized to produce morphogenically active material.

These procedures also can be used to adsorb other active therapeutic drugs, hormones, and various bioactive species to the matrix for sustained release purposes.

The above examples are provided to illustrate the invention but not to limit its scope. Other variants of the invention will be readily apparent to one of ordinary skill in the art and are encompassed by the appended claims.

1. A pharmaceutical composition comprising:
a) a first morphogenic protein;
b) a second morphogenic protein different from the first morphogenic protein; and
c) a pharmaceutically acceptable carrier.

2. The pharmaceutical composition according to claim 1, wherein the first and second morphogenic proteins are independently selected from the group consisting of OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3 (BMP-12), CDMP-2 (BMP-13), CDMP-1 (BMP-14), BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, NEURAL and fragments thereof.

3. The pharmaceutical composition according to claim 1, wherein at least one of the first and second morphogenic proteins comprises a dimeric protein having an amino acid sequence having at least 70% homology within the C-terminal 102-106 amino acids of human OP-1.

4. The pharmaceutical composition according to claim 2, wherein the first morphogenic protein is OP-1 or a fragment thereof and the second morphogenic protein is selected from the group consisting of CDMP-1, CDMP-2, CDMP-3 and fragments thereof.

5. The pharmaceutical composition according to claim 4, wherein the second morphogenic protein is CDMP-1 or a fragment thereof.

6. The pharmaceutical composition according to claim 4, wherein the second morphogenic protein is CDMP-2 or a fragment thereof.

7. The pharmaceutical composition according to claim 4, wherein the second morphogenic protein is CDMP-3 or a fragment thereof.

8. The pharmaceutical composition according to claim 1 further comprising at least one additional morphogenic protein.

9. The pharmaceutical composition of claim 8, wherein the additional morphogenic protein is selected from the group consisting of OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3 (BMP-12), CDMP-2 (BMP-13), CDMP-1 (BMP-14), BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, NEURAL and fragments thereof.

10. The pharmaceutical composition according to claim 1, wherein the second morphogenic protein is present in an amount sufficient to synergistically stimulate the first morphogenic protein.

11. The pharmaceutical composition according to claim 1, wherein the first morphogenic protein is present in an amount sufficient to synergistically stimulate the second morphogenic protein.

12. An implantable device comprising:
a) an implantable biocompatible carrier;
b) a first morphogenic protein; and
c) a second morphogenic protein different from the first morphogenic protein.

13. The device according to claim 12, wherein the biocompatible carrier is a biocompatible matrix.

14. The device according to claim 13, wherein the matrix is selected from the group consisting of demineralized, protein-extracted, particulate, allogenic bone, collagen and calcium phosphate.

15. The device according to any one of claim 12, wherein the first and second morphogenic proteins independently are selected from the group consisting of OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3 (BMP-12), CDMP-2 (BMP-13), CDMP-1 (BMP-14), BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, NEURAL and fragments thereof.

16. The device according to of claim 12, wherein at least one of the first and second morphogenic proteins comprises a dimeric protein having an amino acid sequence having at least 70% homology within the C-terminal terminal 102-106 amino acids of human OP-1.

17. The device according to claim 15, wherein the first morphogenic protein is OP-1or a fragment thereof and the second morphogenic protein is selected from the group consisting of CDMP-1, CDMP-2, CDMP-3 and fragments thereof.

18. The device according to claim 17, wherein the second morphogenic protein is CDMP-1 or a fragment thereof.

19. The device according to claim 17, wherein the second morphogenic protein is CDMP-2 or a fragment thereof.

20. The device according to claim 17, wherein the second morphogenic protein is CDMP-3 or a fragment thereof.

21. The device according to claim 12 further comprising at least one additional morphogenic protein.

22. The device according to claim 21, wherein the additional morphogenic protein is selected from the group consisting of OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3 (BMP-12), CDMP-2 (BMP-13), CDMP-1 (BMP-14), BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, NEURAL and fragments thereof.

23. The device according to claim 12, wherein the second morphogenic protein is present in an amount sufficient to synergistically stimulate the first morphogenic protein.

24. The device according to claim 12, wherein the first morphogenic protein is present in an amount sufficient to synergistically stimulate the second morphogenic protein.

25. A method of improving the tissue inductive activity in a mammal of a first morphogenic protein capable of inducing tissue formation when accessible to a progenitor cell by coadministering an effective amount of at least a second morphogenic protein.

26. The method according to claim 25, wherein the second morphogenic protein synergistically improves the tissue inductive activity of the first morphogenic protein.

27. The method according to claim 25 or 26, wherein the tissue formation is selected from the group consisting of bone, cartilage, tendon and ligament formation.

28. The method according to claim 25, wherein the first and second morphogenic proteins independently are selected from the group consisting of OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3 (BMP-12), CDMP-2 (BMP-13), CDMP-1 (BMP-14), BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, NEURAL and fragments thereof.

29. The method according to claim 25, wherein at least one of the first and second morphogenic proteins comprises a dimeric protein having an amino acid sequence having at least 70% homology within the C-terminal 102-106 amino acids of human OP-1.

30. The method according to claim 28, wherein the first morphogenic protein is OP-1 or a fragment thereof and the second morphogenic protein is selected from the group consisting of CDMP-1, CDMP-2, CDMP-3 and fragments thereof.

31. The method according to claim 28, wherein the second morphogenic protein is CDMP-1 or fragment thereof.

32. The method according to claim 28, wherein the second morphogenic protein is CDMP-2 or fragment thereof.

33. The method according to claim 28, wherein the second morphogenic protein is CDMP-3 or fragment thereof.

34. The method according to claim 25, further comprising a third morphogenic protein.

35. The method according to claim 34, wherein the additional morphogenic protein is selected from the group consisting of OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3 (BMP-12), CDMP-2 (BMP-13), CDMP-1 (BMP-14), BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, NEURAL and fragments thereof.

36. A method of inducing local tissue formation from a progenitor cell in a mammal comprising the step of implanting in the mammal a composition according to claim 1 or a device according to claim 12.

37. The method according to claim 36, wherein the first and second morphogenic proteins are each independently capable of inducing the progenitor cell to form bone, cartilage, tendon or ligament.

38. The method according to claim 36, wherein the second morphogenic protein synergistically improves the tissue inductive activity of the first morphogenic protein.

39. A method for improving the tissue inductive activity in a mammal of a first morphogenic protein capable of inducing tissue formation at a target locus by coadministering an effective amount of a second morphogenic protein, the method comprising the step of:
administering to the target locus a nucleic acid encoding the first morphogenic protein and a nucleic acid encoding the second morphogenic protein.

40. A method for improving the tissue inductive activity in a mammal of a first morphogenic protein capable of inducing tissue formation at a target locus by coadministering an effective amount of a second morphogenic protein, the method comprising the step of:
administering to the target locus a vector comprising a nucleic acid encoding the first morphogenic protein operably linked to an expression control sequence and a vector comprising a nucleic acid encoding the second morphogenic protein operably linked to an expression control sequence.

41. A method for improving the tissue inductive activity in a mammal of a first morphogenic protein capable of inducing tissue formation at a target locus by coadministering an effective amount of a second morphogenic protein, the method comprising the step of:
administering to the target locus a cell comprising a vector comprising a nucleic acid encoding the first morphogenic protein operably linked to an expression control sequence and a cell comprising a vector comprising a nucleic acid encoding the second morphogenic protein operably linked to an expression control sequence.

42. The method according to any one of claims 39-41, wherein the second morphogenic protein synergistically improves the tissue inductive activity of the first morphogenic protein.

43. The method according to any one of claims 39-41, wherein the first morphogenic protein synergistically improves the tissue inductive activity of the second morphogenic protein.

44. The method according to any one of claims 39-41, wherein the tissue formation is selected from the group consisting of bone, cartilage, tendon and ligament formation.

45. The method according to any one of claims 39-41, wherein the first and second morphogenic proteins independently are selected from the group consisting of OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3 (BMP-12), CDMP-2 (BMP-13), CDMP-1 (BMP-14), BMP-15, BMP-16, BMP-17, BMP-18, GDF-1, GDF-2, GDF-3, GDF-5, GDF-6, GDF-7, GDF-8, GDF-9, GDF-10, GDF- 11, GDF-12, MP 121, dorsalin-1, DPP, Vg-1, Vgr-1, 60A protein, NODAL, UNIVIN, SCREW, ADMP, NEURAL and fragments thereof.

46. The method according to any one of claims 39-41, wherein at least one of the first and second morphogenic proteins comprises a dimeric protein having an amino acid sequence having at least 70% homology within the C-terminal 102-106 amino acids of human OP-1.

47. The method according to claim 45, wherein the first morphogenic protein is OP-1 or fragment thereof and the second morphogenic protein is selected from the group consisting of CDMP- 1, CDMP-2, CDMP-3 and fragments thereof.

48. The method according to claim 45, wherein the second morphogenic protein is CDMP-1 or fragment thereof.

49. The method according to claim 45, wherein the second morphogenic protein is CDMP-2 or fragment thereof.

50. The method according to claim 45, wherein the second morphogenic protein is CDMP-3 or fragment thereof.

51. The method according to any one of claims 39-41, further comprising a third morphogenic protein.

52. The method according to claim 51, wherein the additional morphogenic protein is selected from the group consisting of OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3 (BMP-12), CDMP-2 (BMP-13), CDMP-1 (BMP-14), BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, NEURAL and fragments thereof.

53. The method of any one of claims 39-41, wherein the first morphogenic protein and second morphogenic protein are administered simultaneously to the target locus.

54. The method of any one of claims 39-41, wherein the first morphogenic protein and the second morphogenic protein are administered separately to the target locus.

55. The method of claim 40, wherein the nucleic acids encoding the first morphogenic protein and the second morphogenic protein are in the same vector.

56. The method of claim 40, wherein the nucleic acids encoding the first morphogenic protein and the second morphogenic protein are in separate vectors.

57. The method of claim 40, wherein the vectors comprising the nucleic acids encoding the first morphogenic protein and the second morphogenic protein are in the same cell.

58. The method of claim 40, wherein the vectors comprising the nucleic acids encoding the first morphogenic protein and the second morphogenic protein are in separate cells.


SEQUENCE LISTING (18 sequences)

SEQ ID NO: 1 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 2 - protein, 96 aa, Artificial Sequence
  synthetic peptide COP5
LYVDFSDVGWDDWIVAPPGYQAFYCHGECPFPLADHFNSTNHAVVQTLVNSVNSKIPKACCVPTELSAISMLYLDENEKVVLKYNQEMVVEGCGCR

SEQ ID NO: 3 - protein, 96 aa, Artificial Sequence
  synthetic peptide COP7
LYVDFSDVGWNDWIVAPPGYHAFYCHGECPFPLADHLNSTNHAVVQTLVNSVNSKIPKACCVPTELSAISMLYLDENEKVVLKYNQEMVVEGCGCR

SEQ ID NO: 4 - protein, 102 aa, Artificial Sequence
  Formula sequence
  MOD_RES (2): Lys or Arg
  MOD_RES (3): Lys or Arg
  MOD_RES (11): Arg or Gln
  MOD_RES (16): Gln or Leu
  MOD_RES (19): Ile or Val
  MOD_RES (23): Glu or Gln
  MOD_RES (26): Ala or Ser
  MOD_RES (35): Ala or Ser
  MOD_RES (39): Asn or Asp
  MOD_RES (41): Tyr or Cys
  MOD_RES (50): Val or Leu
  MOD_RES (52): Ser or Thr
  MOD_RES (56): Phe or Leu
  MOD_RES (57): Ile or Met
  MOD_RES (58): Asn or Lys
  MOD_RES (60): Glu, Asp or Asn
  MOD_RES (61): Thr, Ala or Val
  MOD_RES (65): Pro or Ala
  MOD_RES (71): Gln or Lys
  MOD_RES (73): Asn or Ser
  MOD_RES (75): Ile or Thr
  MOD_RES (80): Phe or Tyr
  MOD_RES (82): Asp or Ser
  MOD_RES (84): Ser or Asn
  MOD_RES (89): Lys or Arg
  MOD_RES (91): Tyr or His
  MOD_RES (97): Arg or Lys
CXXHELYVSFXDLGWXDWXIAPXGYXAYYCEGECXFPLXSXMNATNHAIXQXLVHXXXPXXVPKXCCAPTXLXAXSVLYXDXSXNVILXKXRNMVVXACGCH

SEQ ID NO: 5 - protein, 97 aa, Artificial Sequence
  Formula sequence
  MOD_RES (2): Tyr or Lys
  MOD_RES (3): Val or Ile
  MOD_RES (4): Ser, Asp or Glu
  MOD_RES (6): Arg, Gln, Ser, Lys or Ala
  MOD_RES (7): Asp or Glu
  MOD_RES (8): Leu, Val or Ile
  MOD_RES (11): Gln, Leu, Asp, His, Asn or Ser
  MOD_RES (12): Asp, Arg, Asn or Glu
  MOD_RES (13): Trp or Ser
  MOD_RES (14): Ile or Val
  MOD_RES (15): Ile or Val
  MOD_RES (16): Ala or Ser
  MOD_RES (18): Glu, Gln, Leu, Lys, Pro or Arg
  MOD_RES (19): Gly or Ser
  MOD_RES (20): Tyr or Phe
  MOD_RES (21): Ala, Ser, Asp, Met, His, Gln, Leu or Gly
  MOD_RES (23): Tyr, Asn or Phe
  MOD_RES (26): Glu, His, Tyr, Asp, Gln, Ala or Ser
  MOD_RES (28): Glu, Lys, Asp, Gln or Ala
  MOD_RES (30): Ala, Ser, Pro, Gln, Ile or Asn
  MOD_RES (31): Phe, Leu or Tyr
  MOD_RES (33): Leu, Val or Met
  MOD_RES (34): Asn, Asp, Ala, Thr or Pro
  MOD_RES (35): Ser, Asp, Glu, Leu, Ala or Lys
  MOD_RES (36): Tyr, Cys, His, Ser or Ile
  MOD_RES (37): Met, Phe, Gly or Leu
  MOD_RES (38): Asn, Ser or Lys
  MOD_RES (39): Ala, Ser, Gly or Pro
  MOD_RES (40): Thr, Leu or Ser
  MOD_RES (44): Ile, Val or Thr
  MOD_RES (45): Val, Leu, Met or Ile
  MOD_RES (46): Gln or Arg
  MOD_RES (47): Thr, Ala or Ser
  MOD_RES (48): Leu or Ile
  MOD_RES (49): Val or Met
  MOD_RES (50): His, Asn or Arg
  MOD_RES (51): Phe, Leu, Asn, Ser, Ala or Val
  MOD_RES (52): Ile, Met, Asn, Ala, Val, Gly or Leu
  MOD_RES (53): Asn, Lys, Ala, Glu, Gly or Phe
  MOD_RES (54): Pro, Ser or Val
  MOD_RES (55): Glu, Asp, Asn, Gly, Val, Pro or Lys
  MOD_RES (56): Thr, Ala, Val, Lys, Asp, Tyr, Ser, Gly, Ile or His
  MOD_RES (57): Val, Ala or Ile
  MOD_RES (58): Pro or Asp
  MOD_RES (59): Lys, Leu or Glu
  MOD_RES (60): Pro, Val or Ala
  MOD_RES (63): Ala or Val
  MOD_RES (65): Thr, Ala or Glu
  MOD_RES (66): Gln, Lys, Arg or Glu
  MOD_RES (67): Leu, Met or Val
  MOD_RES (68): Asn, Ser, Asp or Gly
  MOD_RES (69): Ala, Pro or Ser
  MOD_RES (70): Ile, Thr, Val or Leu
  MOD_RES (71): Ser, Ala or Pro
  MOD_RES (72): Val, Leu, Met or Ile
  MOD_RES (74): Tyr or Phe
  MOD_RES (75): Phe, Tyr, Leu or His
  MOD_RES (76): Asp, Asn or Leu
  MOD_RES (77): Asp, Glu, Asn, Arg or Ser
  MOD_RES (78): Ser, Gln, Asn, Tyr or Asp
  MOD_RES (79): Ser, Asn, Asp, Glu or Lys
  MOD_RES (80): Asn, Thr or Lys
  MOD_RES (82): Ile, Val or Asn
  MOD_RES (84): Lys or Arg
  MOD_RES (85): Lys, Asn, Gln, His, Arg or Val
  MOD_RES (86): Tyr, Glu or His
  MOD_RES (87): Arg, Gln, Glu or Pro
  MOD_RES (88): Asn, Glu, Trp or Asp
  MOD_RES (90): Val, Thr, Ala or Ile
  MOD_RES (92): Arg, Lys, Val, Asp, Gln or Glu
  MOD_RES (93): Ala, Gly, Glu or Ser
  MOD_RES (95): Gly or Ala
  MOD_RES (97): His or Arg
LXXXFXXXGWXXXXXXPXXXXAXYCXGXCXXPXXXXXXXXNHAXXXXXXXXXXXXXXXXXCCXPXXXXXXXXLXXXXXXXVXLXXXXXMXVXXCXCX

SEQ ID NO: 6 - protein, 102 aa, Artificial Sequence
  Formula sequence
  MOD_RES (2): Lys, Arg, Ala or Gln
  MOD_RES (3): Lys, Arg or Met
  MOD_RES (4): His, Arg or Gln
  MOD_RES (5): Glu, Ser, His, Gly, Arg, Pro, Thr or Tyr
  MOD_RES (7): Tyr or Lys
  MOD_RES (8): Val or Ile
  MOD_RES (9): Ser, Asp or Glu
  MOD_RES (11): Arg, Gln, Ser, Lys or Ala
  MOD_RES (12): Asp or Glu
  MOD_RES (13): Leu, Val or Ile
  MOD_RES (16): Gln, Leu, Asp, His, Asn or Ser
  MOD_RES (17): Asp, Arg, Asn or Glu
  MOD_RES (18): Trp or Ser
  MOD_RES (19): Ile or Val
  MOD_RES (20): Ile or Val
  MOD_RES (21): Ala or Ser
  MOD_RES (23): Glu, Gln, Leu, Lys, Pro or Arg
  MOD_RES (24): Gly or Ser
  MOD_RES (25): Tyr or Phe
  MOD_RES (26): Ala, Ser, Asp, Met, His, Gln, Leu or Gly
  MOD_RES (28): Tyr, Asn or Phe
  MOD_RES (31): Glu, His, Tyr, Asp, Gln, Ala or Ser
  MOD_RES (33): Glu, Lys, Asp, Gln or Ala
  MOD_RES (35): Ala, Ser, Pro, Gln, Ile or Asn
  MOD_RES (36): Phe, Leu or Tyr
  MOD_RES (38): Leu, Val or Met
  MOD_RES (39): Asn, Asp, Ala, Thr or Pro
  MOD_RES (40): Ser, Asp, Glu, Leu, Ala or Lys
  MOD_RES (41): Tyr, Cys, His, Ser or Ile
  MOD_RES (42): Met, Phe, Gly or Leu
  MOD_RES (43): Asn, Ser or Lys
  MOD_RES (44): Ala, Ser, Gly or Pro
  MOD_RES (45): Thr, Leu or Ser
  MOD_RES (49): Ile, Val or Thr
  MOD_RES (50): Val, Leu, Met or Ile
  MOD_RES (51): Gln or Arg
  MOD_RES (52): Thr, Ala or Ser
  MOD_RES (53): Leu or Ile
  MOD_RES (54): Val or Met
  MOD_RES (55): His, Asn or Arg
  MOD_RES (56): Phe, Leu, Asn, Ser, Ala or Val
  MOD_RES (57): Ile, Met, Asn, Ala, Val, Gly or Leu
  MOD_RES (58): Asn, Lys, Ala, Glu, Gly or Phe
  MOD_RES (59): Pro, Ser or Val
  MOD_RES (60): Glu, Asp, Asn, Gly, Val, Pro or Lys
  MOD_RES (61): Thr, Ala, Val, Lys, Asp, Tyr, Ser, Gly, Ile or His
  MOD_RES (62): Val, Ala or Ile
  MOD_RES (63): Pro or Asp
  MOD_RES (64): Lys, Leu or Glu
  MOD_RES (65): Pro, Val or Ala
  MOD_RES (68): Ala or Val
  MOD_RES (70): Thr, Ala or Glu
  MOD_RES (71): Gln, Lys, Arg or Glu
  MOD_RES (72): Leu, Met or Val
  MOD_RES (73): Asn, Ser, Asp or Gly
  MOD_RES (74): Ala, Pro or Ser
  MOD_RES (75): Ile, Thr, Val or Leu
  MOD_RES (76): Ser, Ala or Pro
  MOD_RES (77): Val, Leu, Met or Ile
  MOD_RES (79): Tyr or Phe
  MOD_RES (80): Phe, Tyr, Leu or His
  MOD_RES (81): Asp, Asn or Leu
  MOD_RES (82): Asp, Glu, Asn, Arg or Ser
  MOD_RES (83): Ser, Gln, Asn, Tyr or Asp
  MOD_RES (84): Ser, Asn, Asp, Glu or Lys
  MOD_RES (85): Asn, Thr or Lys
  MOD_RES (87): Ile, Val or Asn
  MOD_RES (89): Lys or Arg
  MOD_RES (90): Lys, Asn, Gln, His, Arg or Val
  MOD_RES (91): Tyr, Glu or His
  MOD_RES (92): Arg, Gln, Glu or Pro
  MOD_RES (93): Asn, Glu, Trp or Asp
  MOD_RES (95): Val, Thr, Ala or Ile
  MOD_RES (97): Arg, Lys, Val, Asp, Gln or Glu
  MOD_RES (98): Ala, Gly, Glu or Ser
  MOD_RES (100): Gly or Ala
  MOD_RES (102): His or Arg
CXXXXLXXXFXXXGWXXXXXXPXXXXAXYCXGXCXXPXXXXXXXXNHAXXXXXXXXXXXXXXXXXCCXPXXXXXXXXLXXXXXXXVXLXXXXXMXVXXCXCX

SEQ ID NO: 7 - protein, 97 aa, Artificial Sequence
  Formula sequence
  MOD_RES (1): Phe, Leu or Glu
  MOD_RES (2): Tyr, Phe, His, Arg, Thr, Lys, Gln, Val or Glu
  MOD_RES (3): Val, Ile, Leu or Asp
  MOD_RES (4): Ser, Asp, Glu, Asn or Phe
  MOD_RES (5): Phe or Glu
  MOD_RES (6): Arg, Gln, Lys, Ser, Glu, Ala or Asn
  MOD_RES (7): Asp, Glu, Leu, Ala or Gln
  MOD_RES (8): Leu, Val, Met, Ile or Phe
  MOD_RES (9): Gly, His or Lys
  MOD_RES (10): Trp or Met
  MOD_RES (11): Gln, Leu, His, Glu, Asn, Asp, Ser or Gly
  MOD_RES (12): Asp, Asn, Ser, Lys, Arg, Glu or His
  MOD_RES (13): Trp or Ser
  MOD_RES (14): Ile or Val
  MOD_RES (15): Ile or Val
  MOD_RES (16): Ala, Ser, Tyr or Trp
  MOD_RES (18): Glu, Lys, Gln, Met, Pro, Leu, Arg, His or Lys
  MOD_RES (19): Gly, Glu, Asp, Lys, Ser, Gln, Arg or Phe
  MOD_RES (20): Tyr or Phe
  MOD_RES (21): Ala, Ser, Gly, Met, Gln, His, Glu, Asp, Leu, Asn, Lys or Thr
  MOD_RES (22): Ala or Pro
  MOD_RES (23): Tyr, Phe, Asn, Ala or Arg
  MOD_RES (24): Tyr, His, Glu, Phe or Arg
  MOD_RES (26): Glu, Asp, Ala, Ser, Tyr, His, Lys, Arg, Gln or Gly
  MOD_RES (28): Glu, Asp, Leu, Val, Lys, Gly, Thr, Ala or Gln
  MOD_RES (30): Ala, Ser, Ile, Asn, Pro, Glu, Asp, Phe, Gln or Leu
  MOD_RES (31): Phe, Tyr, Leu, Asn, Gly or Arg
  MOD_RES (32): Pro, Ser, Ala or Val
  MOD_RES (33): Leu, Met, Glu, Phe or Val
  MOD_RES (34): Asn, Asp, Thr, Gly, Ala, Arg, Leu or Pro
  MOD_RES (35): Ser, Ala, Glu, Asp, Thr, Leu, Lys, Gln or His
  MOD_RES (36): Tyr, His, Cys, Ile, Arg, Asp, Asn, Lys, Ser, Glu or Gly
  MOD_RES (37): Met, Leu, Phe, Val, Gly or Tyr
  MOD_RES (38): Asn, Glu, Thr, Pro, Lys, His, Gly, Met, Val or Arg
  MOD_RES (39): Ala, Ser, Gly, Pro or Phe
  MOD_RES (40): Thr, Ser, Leu, Pro, His or Met
  MOD_RES (41): Asn, Lys, Val, Thr or Gln
  MOD_RES (42): His, Tyr or Lys
  MOD_RES (43): Ala, Thr, Leu or Tyr
  MOD_RES (44): Ile, Thr, Val, Phe, Tyr, Met or Pro
  MOD_RES (45): Val, Leu, Met, Ile or His
  MOD_RES (46): Gln, Arg or Thr
  MOD_RES (47): Thr, Ser, Ala, Asn or His
  MOD_RES (48): Leu, Asn or Ile
  MOD_RES (49): Val, Met, Leu, Pro or Ile
  MOD_RES (50): His, Asn, Arg, Lys, Tyr or Gln
  MOD_RES (51): Phe, Leu, Ser, Asn, Met, Ala, Arg, Glu, Gly or Gln
  MOD_RES (52): Ile, Met, Leu, Val, Lys, Gln, Ala or Tyr
  MOD_RES (53): Asn, Phe, Lys, Glu, Asp, Ala, Gln, Gly, Leu or Val
  MOD_RES (54): Pro, Asn, Ser, Val or Asp
  MOD_RES (55): Glu, Asp, Asn, Lys, Arg, Ser, Gly, Thr, Gln, Pro or His
  MOD_RES (56): Thr, His, Tyr, Ala, Ile, Lys, Asp, Ser, Gly or Arg
  MOD_RES (57): Val, Ile, Thr, Ala, Leu or Ser
  MOD_RES (58): Pro, Gly, Ser, Asp or Ala
  MOD_RES (59): Lys, Leu, Pro, Ala, Ser, Glu, Arg or Gly
  MOD_RES (60): Pro, Ala, Val, Thr or Ser
  MOD_RES (61): Cys, Val or Ser
  MOD_RES (63): Ala, Val or Thr
  MOD_RES (65): Thr, Ala, Glu, Val, Gly, Asp or Tyr
  MOD_RES (66): Gln, Lys, Glu, Arg or Val
  MOD_RES (67): Leu, Met, Thr or Tyr
  MOD_RES (68): Asn, Ser, Gly, Thr, Asp, Glu, Lys or Val
  MOD_RES (69): Ala, Pro, Gly or Ser
  MOD_RES (70): Ile, Thr, Leu or Val
  MOD_RES (71): Ser, Pro, Ala, Thr, Asn or Gly
  MOD_RES (72): Val, Ile, Leu or Met
  MOD_RES (74): Tyr, Phe, Arg, Thr, Tyr or Met
  MOD_RES (75): Phe, Tyr, His, Leu, Ile, Lys, Gln or Val
  MOD_RES (76): Asp, Leu, Asn or Glu
  MOD_RES (77): Asp, Ser, Arg, Asn, Glu, Ala, Lys, Gly or Pro
  MOD_RES (78): Ser, Asn, Asp, Tyr, Ala, Gly, Gln, Met, Glu, Asn or Lys
  MOD_RES (79): Ser, Asn, Glu, Asp, Val, Lys, Gly, Gln, or Arg
  MOD_RES (80): Asn, Lys, Thr, Pro, Val, Ile, Arg, Ser or Gln
  MOD_RES (81): Val, Ile, Thr or Ala
  MOD_RES (82): Ile, Asn, Val, Leu, Tyr, Asp or Ala
  MOD_RES (83): Leu, Tyr, Lys or Ile
  MOD_RES (84): Lys, Arg, Asn, Tyr, Phe, Thr, Glu or Gly
  MOD_RES (85): Lys, Arg, His, Gln, Asn, Glu or Val
  MOD_RES (86): Tyr, His, Glu or Ile
  MOD_RES (87): Arg, Glu, Gln, Pro or Lys
  MOD_RES (88): Asn, Asp, Ala, Glu, Gly or Lys
  MOD_RES (89): Met or Ala
  MOD_RES (90): Val, Ile, Ala, Thr, Ser or Lys
  MOD_RES (91): Val or Ala
  MOD_RES (92): Arg, Lys, Gln, Asp, Glu, Val, Ala, Ser or Thr
  MOD_RES (93): Ala, Ser, Glu, Gly, Arg or Thr
  MOD_RES (95): Gly, Ala or Thr
  MOD_RES (97): His, Arg, Gly, Leu or Ser
XXXXXXXXXXXXXXXXPXXXXXXXCXGXCXXXXXXXXXXXXXXXXXXXXXXXXXXXXXXXXCXPXXXXXXXXLXXXXXXXXXXXXXXXXXXXXCXCX

SEQ ID NO: 8 - protein, 102 aa, Artificial Sequence
  Formula sequence
  MOD_RES (2): Lys, Arg, Gln, Ser, His, Glu, Ala or Cys
  MOD_RES (3): Lys, Arg, Met, Lys, Thr, Leu, Tyr or Ala
  MOD_RES (4): His, Gln, Arg, Lys, Thr, Leu, Val, Pro or Tyr
  MOD_RES (5): Gln, Thr, His, Arg, Pro, Ser, Ala, Gln, Asn, Tyr, Lys, Asp or Leu
  MOD_RES (6): Phe, Leu or Glu
  MOD_RES (7): Tyr, Phe, His, Arg, Thr, Lys, Gln, Val or Glu
  MOD_RES (8): Val, Ile, Leu or Asp
  MOD_RES (9): Ser, Asp, Glu, Asn or Phe
  MOD_RES (10): Phe or Glu
  MOD_RES (11): Arg, Gln, Lys, Ser, Glu, Ala or Asn
  MOD_RES (12): Asp, Glu, Leu, Ala or Gln
  MOD_RES (13): Leu, Val, Met, Ile or Phe
  MOD_RES (14): Gly, His or Lys
  MOD_RES (15): Trp or Met
  MOD_RES (16): Gln, Leu, His, Glu, Asn, Asp, Ser or Gly
  MOD_RES (17): Asp, Asn, Ser, Lys, Arg, Glu or His
  MOD_RES (18): Trp or Ser
  MOD_RES (19): Ile or Val
  MOD_RES (20): Ile or Val
  MOD_RES (21): Ala, Ser, Tyr or Trp
  MOD_RES (23): Glu, Lys, Gln, Met, Pro, Leu, Arg, His or Lys
  MOD_RES (24): Gly, Glu, Asp, Lys, Ser, Gln, Arg or Phe
  MOD_RES (25): Tyr or Phe
  MOD_RES (26): Ala, Ser, Gly, Met, Gln, His, Glu, Asp, Leu, Asn, Lys or Thr
  MOD_RES (27): Ala or Pro
  MOD_RES (28): Tyr, Phe, Asn, Ala or Arg
  MOD_RES (29): Tyr, His, Glu, Phe or Arg
  MOD_RES (31): Glu, Asp, Ala, Ser, Tyr, His, Lys, Arg, Gln or Gly
  MOD_RES (33): Glu, Asp, Leu, Val, Lys, Gly, Thr, Ala or Gln
  MOD_RES (35): Ala, Ser, Ile, Asn, Pro, Glu, Asp, Phe, Gln or Leu
  MOD_RES (36): Phe, Tyr, Leu, Asn, Gly or Arg
  MOD_RES (37): Pro, Ser, Ala or Val
  MOD_RES (38): Leu, Met, Glu, Phe or Val
  MOD_RES (39): Asn, Asp, Thr, Gly, Ala, Arg, Leu or Pro
  MOD_RES (40): Ser, Ala, Glu, Asp, Thr, Leu, Lys, Gln or His
  MOD_RES (41): Tyr, His, Cys, Ile, Arg, Asp, Asn, Lys, Ser, Glu or Gly
  MOD_RES (42): Met, Leu, Phe, Val, Gly or Tyr
  MOD_RES (43): Asn, Glu, Thr, Pro, Lys, His, Gly, Met, Val or Arg
  MOD_RES (44): Ala, Ser, Gly, Pro or Phe
  MOD_RES (45): Thr, Ser, Leu, Pro, His or Met
  MOD_RES (46): Asn, Lys, Val, Thr or Gln
  MOD_RES (47): His, Tyr or Lys
  MOD_RES (48): Ala, Thr, Leu or Tyr
  MOD_RES (49): Ile, Thr, Val, Phe, Tyr, Met or Pro
  MOD_RES (50): Val, Leu, Met, Ile or His
  MOD_RES (51): Gln, Arg or Thr
  MOD_RES (52): Thr, Ser, Ala, Asn or His
  MOD_RES (53): Leu, Asn or Ile
  MOD_RES (54): Val, Met, Leu, Pro or Ile
  MOD_RES (55): His, Asn, Arg, Lys, Tyr or Gln
  MOD_RES (56): Phe, Leu, Ser, Asn, Met, Ala, Arg, Glu, Gly or Gln
  MOD_RES (57): Ile, Met, Leu, Val, Lys, Gln, Ala or Tyr
  MOD_RES (58): Asn, Phe, Lys, Glu, Asp, Ala, Gln, Gly, Leu or Val
  MOD_RES (59): Pro, Asn, Ser, Val or Asp
  MOD_RES (60): Glu, Asp, Asn, Lys, Arg, Ser, Gly, Thr, Gln, Pro or His
  MOD_RES (61): Thr, His, Tyr, Ala, Ile, Lys, Asp, Ser, Gly or Arg
  MOD_RES (62): Val, Ile, Thr, Ala, Leu or Ser
  MOD_RES (63): Pro, Gly, Ser, Asp or Ala
  MOD_RES (64): Lys, Leu, Pro, Ala, Ser, Glu, Arg or Gly
  MOD_RES (65): Pro, Ala, Val, Thr or Ser
  MOD_RES (66): Cys, Val or Ser
  MOD_RES (68): Ala, Val or Thr
  MOD_RES (70): Thr, Ala, Glu, Val, Gly, Asp or Tyr
  MOD_RES (71): Gln, Lys, Glu, Arg or Val
  MOD_RES (72): Leu, Met, Thr or Tyr
  MOD_RES (73): Asn, Ser, Gly, Thr, Asp, Glu, Lys or Val
  MOD_RES (74): Ala, Pro, Gly or Ser
  MOD_RES (75): Ile, Thr, Leu or Val
  MOD_RES (76): Ser, Pro, Ala, Thr, Asn or Gly
  MOD_RES (77): Val, Ile, Leu or Met
  MOD_RES (79): Tyr, Phe, Arg, Thr, Tyr or Met
  MOD_RES (80): Phe, Tyr, His, Leu, Ile, Lys, Gln or Val
  MOD_RES (81): Asp, Leu, Asn or Glu
  MOD_RES (82): Asp, Ser, Arg, Asn, Glu, Ala, Lys, Gly or Pro
  MOD_RES (83): Ser, Asn, Asp, Tyr, Ala, Gly, Gln, Met, Glu, Asn or Lys
  MOD_RES (84): Ser, Asn, Glu, Asp, Val, Lys, Gly, Gln, or Arg
  MOD_RES (85): Asn, Lys, Thr, Pro, Val, Ile, Arg, Ser or Gln
  MOD_RES (86): Val, Ile, Thr or Ala
  MOD_RES (87): Ile, Asn, Val, Leu, Tyr, Asp or Ala
  MOD_RES (88): Leu, Tyr, Lys or Ile
  MOD_RES (89): Lys, Arg, Asn, Tyr, Phe, Thr, Glu or Gly
  MOD_RES (90): Lys, Arg, His, Gln, Asn, Glu or Val
  MOD_RES (91): Tyr, His, Glu or Ile
  MOD_RES (92): Arg, Glu, Gln, Pro or Lys
  MOD_RES (93): Asn, Asp, Ala, Glu, Gly or Lys
  MOD_RES (94): Met or Ala
  MOD_RES (95): Val, Ile, Ala, Thr, Ser or Lys
  MOD_RES (96): Val or Ala
  MOD_RES (97): Arg, Lys, Gln, Asp, Glu, Val, Ala, Ser or Thr
  MOD_RES (98): Ala, Ser, Glu, Gly, Arg or Thr
  MOD_RES (100): Gly, Ala or Thr
  MOD_RES (102): His, Arg, Gly, Leu or Ser
CXXXXXXXXXXXXXXXXXXXXPXXXXXXXCXGXCXXXXXXXXXXXXXXXXXXXXXXXXXXXXXXXXCXPXXXXXXXXLXXXXXXXXXXXXXXXXXXXXCXCX

SEQ ID NO: 9 - protein, 5 aa, Artificial Sequence
  N-term formula peptide fragment
  MOD_RES (2): Lys, Arg, Ala or Gln
  MOD_RES (3): Lys, Arg or Met
  MOD_RES (4): His, Arg or Gln
  MOD_RES (5): Glu, Ser, His, Gly, Arg, Pro, Thr or Tyr
CXXXX

SEQ ID NO: 10 - DNA, 24 bp, Artificial Sequence
  synthetic probe
gccgtgcgtacttagacatgcatg

SEQ ID NO: 11 - DNA, 1814 bp, Homo sapiens
gcccggagcccggagcccgggtagcgcgtagagccggcgcgatgcacgtgcgctcactgcgagctgcggcgccgcacagcttcgtggcgctctgggcacccctgttcctgctgcgctccgccctggccgacttcagcctggacaacgaggtgcactcgagcttcatccaccggcgcctccgcagccaggagcggcgggagatgcagcgcgagatcctctccattttgggcttgccccaccgcccgcgcccgcacctccagggcaagcacaactcggcacccatgttcatgctggacctgtacaacgccatggcggtggaggagggcggcgggcccggcggccagggcttctcctacccctacaaggccgtcttcagtacccagggcccccctctggccagcctgcaagatagccatttcctcaccgacgccgacatggtcatgagcttcgtcaacctcgtggaacatgacaaggaattcttccacccacgctaccaccatcgagagttccggtttgatctttccaagatcccagaaggggaagctgtcacggcagccgaattccggatctacaaggactacatccgggaacgcttcgacaatgagacgttccggatcagcgtttatcaggtgctccaggagcacttgggcagggaatcggatctcttcctgctcgacagccgtaccctctgggcctcggaggagggctggctggtgtttgacatcacagccaccagcaaccactgggtggtcaatccgcggcacaacctgggcctgcagctctcggtggagacgctggatgggcagagcatcaaccccaagttggcgggcctgattgggcggcacgggccccagaacaagcagcccttcatggtggctttcttcaaggccacggaggtccacttccgcagcatccggtccacggggagcaaacagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccactagctcctccgagaattcagaccctttggggccaagtttttctggatcctccattgctcgccttggccaggaaccagcagaccaactgccttttgtgagaccttcccctccctatccccaactttaaaggtgtgagagtattaggaaacatgagcagcatatggcttttgatcagtttttcagtggcagcatccaatgaacaagatcctacaagctgtgcaggcaaaacctagcagaaaaaaaaaacaacgcataaagaaaaatggccgggccaggtcattggctgggaagtctcagccatgcacggactcgtttccagaggtaattatgagcgcctaccagccaggccacccagccgtgggaggaagggggcgtggcaaggggtgggcacattggtgtctgtgcgaaaggaaaattgacccggaagttcctgtaataaatgtcacaataaaacgaatgaatgaaaaaaaaaaaaaaaaa

SEQ ID NO: 12 - DNA, 2323 bp, Homo sapiens
tcaagaacgagttattttcagctgctgactggagacggtgcacgtctggatacgagagcatttccactatgggactggatacaaacacacacccggcagacttcaagagtttcagactgaggagaaaacctttccttctgctgctactgctgctgccgctgcttttgaaagtccactcctttcatggtttttcctgccaaaccagaggcaccttcgctgctgccgctgttctctttggtgtcattcagcggctggccagaggatgagactccccaaactcctcactttcttgctttggtacctggcttggctggacctggaattcatctgcactgtgttgggtgcccctgacttgggccagagaccccaggggtccaggccaggattggccaaagcagaggccaaggagaggccccccctggcccggaacgtcttcaggccagggggtcacagctatggtgggggggccaccaatgccaatgccagggcaaagggaggcaccgggcagacaggaggcctgacacagcccaagaaggatgaacccaaaaagctgccccccagaccgggcggccctgaacccaagccaggacaccctccccaaacaaggcaggctacagcccggactgtgaccccaaaaggacagcttcccggaggcaaggcacccccaaaagcaggatctgtccccagctccttcctgctgaagaaggccagggagcccgggcccccacgagagcccaaggagccgtttcgcccaccccccatcacaccccacgagtacatgctctcgctgtacaggacgctgtccgatgctgacagaaagggaggcaacagcagcgtgaagttggaggctggcctggccaacaccatcaccagctttattgacaaagggcaagatgaccgaggtcccgtggtcaggaagcagaggtacgtgtttgacattagtgccctggagaaggatgggctgctgggggccgagctgcggatcttgcggaagaagccctcggacacggccaagccagcggtcccccggagccggcgggctgcccagctgaagctgtccagctgccccagcggccggcagccggccgccttgctggatgtgcgctccgtgccaggcctggacggatctggctgggaggtgttcgacatctggaagctcttccgaaactttaagaactcggcccagctgtgcctggagctggaggcctgggaacggggcaggaccgtggacctccgtggcctgggcttcgaccgcgccgcccggcaggtccacgagaaggccctgttcctggtgtttggccgcaccaagaaacgggacctgttctttaatgagattaaggcccgctctggccaggacgataagaccgtgtatgagtacctgttcagccagcggcgaaaacggcgggccccatcggccactcgccagggcaagcgacccagcaagaaccttaaggctcgctgcagtcggaaggcactgcatgtcaacttcaaggacatgggctgggacgactggatcatcgcaccccttgagtacgaggctttccactgcgaggggctgtgcgagttcccattgcgctcccacctggagcccacgaatcatgcagtcatccagaccctgatgaactccatggaccccgagtccacaccacccacctgctgtgtgcccacgcggctgagtcccatcagcatcctcttcattgactctgccaacaacgtggtgtataagcagtatgaggacatggtcgtggagtcgtgtggctgcaggtagcagcactggccctctgtcttcctgggtggcacatcccaagagccccttcctgcactcctggaatcacagaggggtcaggaagctgtggcaggagcatctacacagcttggtgaagggattcaataagcttgctcgctctctgagtgtgacttgggctaaaggcccccttttatccacaagttcccctggctgaggattgctgcccgtctgctgatgtgaccagtggcaggcacaggtccagggagacagactctgaatgggactgagtcccaggaaacagtgctttccgatgagactcagcccaccatttctcctcacctgggccttctcagcctctggactctcctaagcacctctcaggagagccacaggtgccactgcctcctcaaatcacatttgtgcctggtgacttcctgtccctgggacagttgagaagctgactgggcaagagtgggagagaagaggagagggcttggatagagttgaggagtgtgaggctgttagactgttagatttaaatgtatattgatgagataaaaagcaaaactgtgccta

SEQ ID NO: 13 - protein, 501 aa, Homo sapiens [sequence omitted]

SEQ ID NO: 14 - DNA, 3532 bp, Homo sapiens
gaggagcgcggcccccgccgctgtccagccgctccgtgcctttcgcgcccggcgcagccgctgctgtcgccgccgcctctccgagtaacttagccactcgctcgccatggacactcctagggtccttctctgggcgatcttcctcattagcttcctctgggatttgccgggtttccagcaggcttccatctcctcgtcctcctcctcctccaccgaattggactccaccaaagacgtggggaaccgcaaagaagggaagatgcagcgaactccacaagagagtgccgagggccggacgcccccggagcacgggctgcggcagaaggacctccggcggcggccgcccggacaacatcaggggcaggagccgccgggcagggggctgcgcgtggtgcctcacgagtacatgctgtcaatctacaagacttactccattgccgagaagctgggcatcaatgccagctttttccagtcttccaagtcagctaatacgatcactagctttgtagacagaggactggacgatctctcgcacactcctctccggagacagaagtatttgtttgatgtgtccacactctcagacaaagaagagctggtgggcgcagagctaaggctttatcgccaggcgcccccaacgccctgggggctaccggcccgaccgctgcacttgcagctcttcccttgtttatccccattgctactggacgccaggaccctggatcctcagggaccaacccaggccggctgggaagtcttcgacgtgtggcagggcctgcgccctcagccttggaagcagctgtgcctggagttgcgggcagcctggggtgagctggacgccggggatacgggggcgcgcgcgaggggtccccagcagccaccgcctctggacctgcggagtctgggcttcggtcggagggtgaggccgccccaggagcgcgccctgcttgtagtgttcaccagatcgcagcgcaagaacctgttcactgagatgcatgagcagctgggctctgcagaggctgcgggagccgaggggtcatggccagcgccgtcgggctccccagacgccgggtcttggctgccctcgcccggccgccggcggcgacgcaccgccttcgccagccgtcacggcaagcgacatggcaagaagtccaggctgcgctgcagcagaaagcctctgcacgtgaattttaaggagttaggctgggacgactggattatcgcgcccctagagtacgaggcctatcactgcgagggcgtgtgcgactttccgctgcgctcgcaccttgagcccactaaccatgccatcattcagacgctgatgaactccatggacccgggctccaccccgcctagctgctgcgttcccaccaaactgactcccattagcatcctgtacatcgacgcgggcaataatgtagtctacaagcagtatgaggacatggtggtggagtcctgcggctgtaggtagcggtgctgtcccgccacctgggccagggaccatggagggaggcctgactgccgagaaaggagcaggagctggcttggaagaggccacaggtgggggacagcctgaaagtaggagcacagtaagaagcagcccagccttcccagaaccttccaacccccaacccagaagcagctaagggtttcacactttgccttgccagcctggaaagactagacaagaaggattctttcttttattatggctttggttctttgtttgttttgttttgttttatctccttggttttgataggacgtggaaggaggggaaatgcttatctgttcacaagtgttctgtggattgggggaggagggaataagagaaataacctattacttttgtccgcatctcagattggttgtttctacctgtgtaagaaagtgaggcctttgggtctttctagccaagtctcagctggcgtccttgacctcacaggaagtatttgaccaaaggaataagtgagtcaggaagggtcttacccttagagaagcccaagctgcctccttctctgctctgtgcatccccacgaatttatcagtctgtcctggtctctcccagcggagggaacagggagttgccaccgctgtgggctcagacctccagatgccttttctgttggtggtggggggggggggggcagaaggatttggtatggatggaatgacaagaattagtccgtgtggaaccccgtgaaggataatgagaaaccacaaggcctacctctgaccgggtgacacagaggcgcattacccaaggctggggtagcccacccaaaagctcttggtcatggatgtttctcagtggcttctttaaacagaatttgccaaaatcccagcatctccttctaaggcaatttttttccctccttaaatggagaggggtggaaactctataattaaggtaagaagggaagaggaggaggaggaaaagacctgaaaggtggaagggctacatcaggaggggagatgcgggccccacaagaggatggagataaagggccaggccccaagcatggaaggaggatgaaagggccaggagtctctgcctctgaagtttcttaaaatttaagtcccctctcctctttctgacattcctgagagattaccagccagcaatagcccagggctccccctagagaaacattttagatcgtaattaccagttaggtgctgtttttaaaccttagtaatgaggagctgtgagtcgagggttacattgagtttggggtggaaactggggtacaggggcaccaggaaagaaaccagaaggaagtaagagacagaacacgggaaatacctgtagtactccaaatctccatcctttcccacagtctgtaccatgattattttcaaacatcgcctagacagagtctctttgagaagtaagcttcctcatgtttgtccatggaacggtttaaactgaggaccctgagtgagtgattacagactgatctgggttcccccaactggtttgctctctctccccgctgaaatctgtttcttgtctggttcagagaagacaaactgtcaaggaaacggtcggaatgctaatactggtaatataaactggtaagggagaaaagacaagacttggccaggagaaataacttaaatgcacatttgctttggatgcactgctgttctgttgaggctgtatatatttgtttatttaagatgactgaaagtgcaaagagaaaacatgcaattcatcttaacgtacaaaacgttatatgcactcaaatgttataatttctaatatttttaaagtttatattcgagttgtacaaagttaagcattaatcagatatttcattctttcataatgttatcattttcttaaatattattacaaaattttaagtgtgtctaatggagagttttttttttgaaactgtctacctcactataatacagattttgacaacactaaagttactggaggtcgattgatatacaaaacatttttacagtataccttcttggacgataaggaatctggttaattctgtcttattaaactcat

SEQ ID NO: 15 - protein, 454 aa, Homo sapiens
MDTPRVLLWAIFLISFLWDLPGFQQASISSSSSSSTELDSTKDVGNRKEGKMQRTPQESAEGRTPPEHGLRQKDLRRRPPGQHQGQEPPGRGLRVVPHEYMLSIYKTYSIAEKLGINASFFQSSKSANTITSFVDRGLDDLSHTPLRRQKYLFDVSTLSDKEELVGAELRLYRQAPPTPWGLPARPLHLQLFPCLSPLLLDARTLDPQGPTQAGWEVFDVWQGLRPQPWKQLCLELRAAWGELDAGDTGARARGPQQPPPLDLRSLGFGRRVRPPQERALLVVFTRSQRKNLFTEMHEQLGSAEAAGAEGSWPAPSGSPDAGSWLPSPGRRRRRTAFASRHGKRHGKKSRLRCSRKPLHVNFKELGWDDWIIAPLEYEAYHCEGVCDFPLRSHLEPTNHAIIQTLMNSMDPGSTPPSCCVPTKLTPISILYIDAGNNVVYKQYEDMVVESCGCR

SEQ ID NO: 16 - DNA, 1456 bp, Homo sapiens
cccggagccacggagcccgcgccgcccgcccgcccggcccacggagcccatggacctgagcgccgccgccgcgctgtgcctttggctgctgagcgcctgccgcccccgcgacgggctggaagcggccgccgtgctgcgagcggcgggggctgggccggtccggagcccagggggcggcggcggcggcggcggcggcggcggcggcgggcggactcttgcccaggctgcgggcgccgcggctgtcccggccgccgcggttccccgggcccgcgccgcgcgccgcgccgcgggctccggcttcaggaacggctcggtggtgccgcaccacttcatgatgtcgctttaccggagcctggccgggagggctccggccggggcagccgctgtctccgcctcgggccatggtcgcgcggacacgatcaccggcttcacagaccaggcgacccaagacgaatcggcagccgaaacaggccagagcttcctgttcgacgtgtccagccttaacgacgcagacgaggtggtgggtgccgagctgcgcgtgctgcgccggggatctccagagtcgggcccaggcagctggacttctccgccgttgctgctgctgtccacgtgcccgggcgccgcccgagcgccacgcctgctgtactcgcgggcagctgagcccctagtcggtcagcgctgggaggcgttcgacgtggcggacgccatgaggcgccaccgtcgtgaaccgcgccccccccgcgcgttctgcctcttgctgcgcgcagtggcaggcccggtgccgagcccgttggcactgcggcgactgggcttcggctggccgggcggagggggctctgcggcagaggagcgcgcggtgctagtcgtctcctcccgcacgcagaggaaagagagcttattccgggagatccgcgcccaggcccgcgcgctcggggccgctctggcctcagagccgctgcccgacccaggaaccggcaccgcgtcgccaagggcagtcattggcggccgcagacggaggaggacggcgttggccgggacgcggacagcgcagggcagcggcgggggcgcgggccggggccacgggcgcaggggccggagccgctgcagccgcaagccgttgcacgtggacttcaaggagctcggctgggacgactggatcatcgcgccgctggactacgaggcgtaccactgcgagggcctttgcgacttccctttgcgttcgcacctcgagcccaccaaccatgccatcattcagacgctgctcaactccatggcaccagacgcggcgccggcctcctgctgtgtgccagcgcgcctcagccccatcagcatcctctacatcgacgccgccaacaacgttgtctacaagcaatacgaggacatggtggtggaggcctgcggctgcaggtagcgcgcgggccggggagggggcagccacgcggccgaggatccccag

SEQ ID NO: 17 - protein, 453 aa, Homo sapiens [sequence omitted]

SEQ ID NO: 18 - protein, 5 aa, Artificial Sequence
  N-term formula peptide fragment
  MOD_RES (2): Lys, Arg, Gln, Ser, His, Glu, Ala or Cys
  MOD_RES (3): Lys, Arg, Met, Lys, Thr, Leu, Tyr or Ala
  MOD_RES (4): His, Gln, Arg, Lys, Thr, Leu, Val, Pro or Tyr
  MOD_RES (5): Gln, Thr, His, Arg, Pro, Ser, Ala, Gln, Asn, Tyr, Lys, Asp or Leu
CXXXX